(19) United States
(12) Patent Application Publication
(10) Pub. No.: US 2024/0376427 A1
(43) Pub. Date: Nov. 14, 2024

(54) COMPOSITIONS AND SYSTEMS COMPRISING THREE-DIMENSIONAL NERVE CELL CULTURES AND METHODS OF USING THE SAME

(71) Applicant: THE ADMINISTRATORS OF THE TULANE EDUCATIONAL FUND, New Orleans, LA (US)

(72) Inventors: Michael James MOORE, New Orleans, LA (US); Kevin J. POLLARD, New Orleans, LA (US)

(21) Appl. No.: 18/687,179

(22) PCT Filed: Aug. 29, 2022
(86) PCT No.: PCT/US22/41938

§ 371 Date: Feb. 27, 2024

Related U.S. Application Data
(60) Provisional application No. 63/238,092, filed on Aug. 27, 2021.

(51) Int. Cl. C12N 5/0793; C12N 5/071; C12N 5/079; G01N 33/50

(52) U.S. Cl. CPC C12N 5/0619; C12N 5/0622; C12N 5/0697; G01N 33/5058; C12N 2502/081; C12N 2513/00

(57) ABSTRACT

The disclosure relates to a system and method of using the system to detect and monitor afferent synaptic nerve fiber function in vitro. The disclosure also relates to a method of screening for test agents or compounds that modulate nerve function, such as test agents that modulate pain sensation in a human subject, by exposing one or a plurality of test agents to systems comprising a first and second spheroid, wherein the first spheroid comprise cells from a mammalian dorsal root ganglia and the second spheroid comprises cells from a mammalian spinal cord.

### CROSS-REFERENCE TO RELATED APPLICATION

This application claims priority to and the benefit of the filing date of U.S. Provisional Patent Application No. 63/238,092, filed Aug. 27, 2021, the entirety of which, including the appendices, is hereby incorporated by reference herein.

### STATEMENT REGARDING FEDERALLY SPONSORED RESEARCH

This invention was made with government support under grant number NIH Grant Nos. UG3-TR003150 and R42-TR001270. The United States government has certain rights in this invention.

### FIELD

The present disclosure generally relates to a cell culturing system, and specifically to a three-dimensional cell culturing system comprising spheroids that promotes both structural and functional characteristics that mimic those of afferent in vivo nerve fibers between brain spheroids and spinal cord spheroids, including the functional characteristics of sensory neurons involved in pain sensation.

### BACKGROUND

Chronic pain is a debilitating and increasingly prevalent condition that continues to be insufficiently managed with modern analgesics (1-5). Many leading analgesics have proven highly addictive and/or ineffective for long-term use, and overreliance on powerful and addictive opioid compounds has contributed to an epidemic of prescription drug abuse in the last decade (6, 7). Conventional preclinical drug screening in animal models has failed to meet the urgent need for novel, safe analgesics and there is increasing recognition of the need for alternative, more efficient preclinical research models (8-12). Currently, nearly 92% of neurological drugs that reach Phase I clinical trials fail to reach the market due to unacceptable toxicity or lack of efficacy in humans (9). Microphysiological systems (MPS) have potential to enhance efficiency of preclinical drug screening by combining the physiologically relevant functional data, normally obtained through in vivo experimentation, with the heightened throughput and experimental control of conventional in vitro methodology (13-19). Future applications of human pluripotent stem cell (hPSC)-derived cell types MPS may even eliminate interspecies differences that confound translational studies and revolutionize patient-specific disease modeling and therapeutic design (20).

### SUMMARY OF EMBODIMENTS

Within the context of MPS, emergent physiology has been described as concerted, macroscopic biological functions that arise through self-directed organization of individual cells into multicellular, cohesive tissues through interactions between cells and with the extracellular environment (21). Identification of emergent physiology with immediate clinical relevance will maximize the current potential of MPS-based drug screening and defining the mechanisms underlying their emergence will inform continued and future development of hPSC-derived MPS.

In vivo, pain processing requires coordinated communication between the peripheral and central nervous systems (PNS and CNS). Pain signals originate in dorsal root ganglion (DRG)-derived nerve fibers of the PNS. Neuronal soma in the DRG project a single pseudounipolar axon, with one branch innervating peripheral tissue and the other innervating the spinal cord. Axon extensions in peripheral tissue detect noxious stimuli and encode that information as a bioelectric pain signal, which propagates along the axon through the DRG towards the spinal cord and is relayed to the CNS through synaptic transmission in the spinal cord dorsal horn (SCDH). Peripheral pain fibers mainly innervate interneurons of the most superficial layers of the SCDH and their input is largely glutamatergic and excitatory (22). The superficial SCDH interneuron population is a mixture of excitatory glutamatergic and inhibitory GABAergic neurons that form a complex, interconnected, polysynaptic circuit that may gate or amplify afferent pain signals en route to ascending projection neurons located in laminae I and III-V (23). Projection neurons extend axons out of the spinal cord along the spinothalamic tract to relay the output of the SCDH circuitry to higher-level brain regions to process the location and emotional aspects of pain (3, 24). Here, we validate an advanced microphysiological model of the DRG-SCDH synaptic circuitry demonstrating unidirectional, concerted synaptic communication transmitted long-distance between independent neurospheroid populations connected only by directed axonal nerve fiber growth. This is distinct from state-of-the-art, fused organoid (“assembloid”) cultures in both the scale of tissue organization and degree of concerted, discrete, population-level physiological function (34, 35). Furthermore, microphysiological transmission was differentially altered by common analgesics consistent with their distinct mechanisms of action. This rodent DRG-SCDH microphysiological co-culture has immediate potential for physiological screening of novel analgesics and defines key emergent behaviors critical for understanding future hPSC-derived microphysiological models of lower afferent pain signaling.

The present disclosure relates to a system or microphysiological models of the nervous system in which a DRG spheroid or spheroid comprising cells from dorsal root ganglia is positioned proximate to a spinal cord tissue or a spheroid comprising cells of the spinal cord that provides 3D architecture as well as specified organization. Other model systems tend to allow only one or the other. Organotypic tissue slices can provide 3D architecture as well as organization specified by nature, but these models are not amenable to very high-throughput analysis.

The disclosure relates to a composition comprising a first spheroid of cells comprising one or a combination of cells and/or tissues chosen from: a neuronal cell, an astrocyte, a glial cell or a combination of two or all three of the aforementioned cell types. In some embodiments, this spheroid is free of dorsal root ganglia or cells derived from a dorsal root ganglia. In some embodiments, the composition comprises a first spheroid of cells comprising tissue from a mammalian spinal cord that is free of cells from a mammalian dorsal root ganglia. In some embodiments, the composition comprises a first spheroid in functional contact with afferent nerve fibers or three dimensional axon bundles. In some embodiments, the bundle is a group of from about 3 axons to about 150 or more axons. In some embodiments, the first spheroid is in operable contact with a second spheroid that comprises cells from a dorsal root ganglia via afferent nerve fibers or axon bundles from the second spheroid of cells to the first spheroid of cells. In some embodiments, the composition of second spheroid of cells comprises one or a combination of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, an astrocyte, an oligodendrocyte, a Schwann cell, a microglial cell, an ependymal cell, a radial glial cell, a satellite cell, an enteric glial cell, a pituicyte, and combinations thereof. In some embodiments, the second spheroid or first spheroid comprise one or a combination of cells chosen from: an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, an oligodendrocyte, a Schwann cell, a microglial cell, an ependymal cell, a radial glial cell, a satellite cell, and a pituicyte.

The disclosure also relates to a system or composition comprising a solid support onto which the one or plurality of spheroids are positioned. In some embodiments, the system is designed for tissue culture growth and propagation of cells within the first and/or second spheroids such that axons grow from the second spheroid to the first spheroid in a channel. In some embodiments, the system or composition comprises the first spheroid and/or second spheroid positioned within the solid support which comprises a hydrogel matrix. The disclosure also relates to a system or composition, wherein the composition comprises a solid support and the solid support comprises hydrogel with a channel positioned between and in fluid communication with a first and second cavities, chambers or vessels; the first volume or cavity comprising the first spheroid and the second volume or cavity comprising the second spheroid, with a channel or path positioned between the first and second cavities such that the first and second cavities are in fluid communication. In some embodiments, the system or composition further comprises a tissue culture medium. In some embodiments, the first spheroid is in electrical communication with the second spheroid by a three-dimensional bundle of axons.

The disclosure relates to a system or composition comprising a bundle of axons operably connecting a first spheroid with a second spheroid, the first spheroid comprising cells from a spinal cord, human cells derived from human stem cells, or mammalian spinal cord tissue. In some embodiments, the spheroid are operably connected by a bundle of axons with from about 3 to about 125 axons. In some embodiments, the spheroid are operably connected by a bundle of axons with from about 10 to about 140 axons. In some embodiments, the spheroid are operably connected by a bundle of axons with from about 25 to about 125 axons. In some embodiments, the spheroid are operably connected by a bundle of axons with from about 30 to about 125 axons. In some systems and/or compositions of the disclosure the first spheroid has a diameter from about 10 to about 15 microns in width. In some embodiments, the second spheroid of cells has a diameter from about 15 to about 20 microns.

The disclosure relates to modified cells in tissue culture, wherein at least one spheroid comprises an exogenous nucleic acid sequence encoding a rhodopsin protein from algae. In some embodiments, the rhodopsin is a channel rhodopsin molecule that is optically excitable by a wavelength of light from about 400 to about 500 nanometers. In some embodiments, the rhodopsin is channel rhodopsin1 (CH1) or channel rhodopsin2 (CH2).

The disclosure also relates to methods of making and using the system and compositions disclosed herein. For instance, the disclosure relates to a method of modulating glutamatergic neurotransmission and/or calcium influx in a spheroid of cells comprising cells derived from spinal cord of a mammal, the method comprising:
(a) exposing a first spheroid of cells derived from dorsal root ganglia of a mammal to a wavelength of light; and
(b) measuring calcium flux across the membrane of one or a plurality of cells in a second spheroid of cells derived from spinal cord; wherein the first and second spheroid of cells are in electrical communication via a plurality of axons connecting the first spheroid with the second spheroid; wherein the first and second spheroid of cells are in fluid communication by a channel connecting a first cavity comprising the first spheroid of cells to a second cavity comprising the second spheroid of cells; wherein the first spheroid of cells comprises an exogenous nucleic acid sequence encoding a channel rhodopsin. Light can excite the second spheroid with central nervous capability and can transmute a signal measurable at the first spheroid. If a control is used to measure what is considered a baseline or normal signal, changes to signal may be recorded or measured under conditions that may alter the optical excitability of the spheroids of cells. In some embodiments, for instance, the method further comprises exposing the system or composition to a test agent and measuring the change, if any, in photosensitivity as compared to the normal or baseline pattern observed in the absence of the test agent. In some embodiments, the test agent is a drug/small molecule, potentially toxic molecule or therapeutic biologic candidate for the treatment of a mammalian subject, such as a human patient.

In some embodiments, the disclosure relates to a method of detecting glutamatergic neurotransmission and/or calcium influx in a spheroid of cells in culture, the method comprising:
(a) exposing a first spheroid of cells to a wavelength of light; and
(b) measuring calcium flux across the membrane of one or a plurality of cells in a second spheroid of cells positioned distally from the first spheroid of cells; wherein the first and second spheroid of cells are in electrical communication via a plurality of axons connecting the first spheroid with the second spheroid; wherein the first and second spheroid of cells are in fluid communication by a channel connecting a first cavity comprising the first spheroid of cells to a second cavity comprising the second spheroid of cells. In some embodiments, the first spheroid of cells comprises an exogenous nucleic acid sequence encoding a channel rhodopsin. In some embodiments, the first spheroid of cells comprises one or a combination of cells chosen from: a glial cell, an embryonic cell, a mesenchymal stem cell, a cell derived from an induced pluripotent stem cell, a sympathetic neuron, a parasympathetic neuron, a trigeminal ganglion, an astrocyte, an oligodendrocyte, a Schwann cell, a microglial cell, an ependymal cell, a radial glial cell, a satellite cell, an enteric glial cell, a pituicyte, and combinations thereof. In some embodiments, the first spheroid of cells, the second spheroid of cells, or the first and second spheroids of cells comprise or consist essentially of human cells.

The disclosure relates to methods, wherein the composition, solid support to system comprises a solid substrate onto which the hydrogel matrix is crosslinked. In some embodiments, the solid substrate comprises a contiguous exterior surface and an interior surface, such solid substrate comprising at least one portion in a cylindrical or substantially cylindrical shape and at least one hollow interior defined at its edge by at least one portion of the interior surface, said interior surface comprising one or a plurality of pores from about 0.1 microns to about 1.0 microns in diameter wherein the hollow interior of the solid substrate is accessible from a point exterior to the solid substrate through at least one opening; wherein the hollow interior portion comprises a first portion proximate to the opening and at least a second portion distal to the opening, each of the first and second portions defining a cavity; wherein the first or second spheroids are positioned at or proximate to the first portion of the hollow interior and are in physical contact with the hydrogel matrix, and wherein the second portion of the at least one hollow interior is in fluid communication with the first portion such that axons are capable of growth from the first cavity into the second interior portion of the hollow interior.

In some embodiments, the hydrogel comprises at least a first cell-impenetrable polymer and a first cell-penetrable polymer. In some embodiments, the at least one cell-impenetrable polymer comprises no greater than about 15% PEG and the at least one cell-penetrable polymer comprises from about 0.05% to about 1.00% of one or a combination of self-assembling peptides chosen from: RAD 16-I, RAD 16-II, EAK 16-I, EAK 16-II, and dEAK 16.

In some embodiments, the composition or system is free of polyethylene glycol (PEG). The disclosure relates to methods of using or a system or composition comprising a solid support comprising a hydrogel, wherein the hydrogel comprises a first region and a second region, the first region is formed in the shape of a cylinder or rectangular prism oriented with its longitudinal axis passing through the top and bottom of the cell culture vessel and each of either the cylinder or rectangular prism comprising a the hollow interior defined by an inner surface of the cylinder or rectangular prism, said hollow interior accessible by one or more openings through the top of the composition; wherein the second region comprises a space formed in the shape of its interior walls with an opening on its side adjacent to and in fluid communication with the first region. In any of the disclosed methods, the methods further comprise a step of seeding the first and second spheroids within a solid substrate and exposing the first and second spheroids to tissue culture media within the disclosed compositions or systems prior to the step of exposing the first spheroid to light or voltage drop, current, test agent or other stimulus.

The disclosure relates to a method of measuring or detecting a recording in an in vitro synapse within a composition comprising: (i) a first spheroid of cells in electrical communication with a second spheroid of cells; (ii) a first electrode proximate to the first spheroid; and (iii) a second electrode proximate to the second spheroid, the method comprising: (a) applying a voltage across the first and second electrodes at the first electrode for a sufficient time period and with a sufficient voltage to generate a detectable control waveform from the second electrode; (b) detecting the control waveform from the second electrode.

The disclosure relates to a method of measuring a waveform from a spheroid in culture with afferent nerve fiber comprising axons produced by exposing the spheroid to a stimulus, such as a voltage potential across it and a second spheroid proximate to an electrode. In some embodiments, the disclosed methods comprise detecting a control waveform from the first spheroid, wherein the control waveform comprises a first and a second, negative-going, or negative amplitude wave, followed by a third positive-going or positive amplitude wave. In some embodiments, the first wave has a duration of its peak from about 1 to about 10 microseconds and a negative amplitude in field potential from about 40 to about 100 microvolts over its duration; wherein the second wave has a duration of its peak from about 1 to about 7 microseconds and a negative amplitude in field potential from about 25 to about 75 microvolts over its duration; and wherein the slower positive-going wave has a peak from about 40 to about 70 microseconds and a positive amplitude in field potential from about 70 to about 250 microvolts. In some embodiments, the methods disclosed herein further comprise applying a second voltage across the first and second spheroids at the first electrode in the presence or absence of a test agent after establishing and repeating exposure to create the control wave. In some embodiments, the methods are free of a step in which the spheroid are exposed to a first or second stimulus within 5, 6, 7, 8, 9, or 10 seconds of each other. In some embodiments, a control wave is produced by exposing the spheroids in culture with a series of electrical stimuli no less than about 10 seconds apart. In some embodiments, the control waveform is produced by applying a voltage potential to a second electrode at or proximate to a second spheroid in culture and subsequently measuring the waveform transmuted to the first spheroid through a first electrode at or proximate to the first spheroid. In some embodiments, each electrode is positioned from about 1 to about 5 mm from its respective spheroid. In some embodiments, the electrode is positioned about 2 mm from each spheroid. In some of the embodiments, the voltage of step (a) is from about 1 to about 50 Volts and repeated once, twice or three times or more before exposure to any test agent or stimulus. In some embodiments, the voltage of step (a) is from about 1 to about 50 Volts and repeated once, twice or three times or more before exposure to any test agent or stimulus for a pulse at 200 microseconds or less at 1, 2, 3, 4 or more Hz.

In some embodiments, a second voltage potential is applied across the first and second electrodes at the first electrode after exposing the spheroids to one or a plurality of test agents. In some embodiments, the methods of the disclosure further comprise a step of detecting a waveform associated with the field potential across the spheroids in the presence of the test agent. Some methods further provide for a step of comparing the second waveform to the control waveform and normalizing or measuring the change in voltage potential. Some methods further provide for a step of correlating the change in voltage potential to the efficacy or neuromodulatory effect of the test agent. In some embodiments, the test agent is a small molecule therapeutic candidate for treating pain.

In some embodiments, the disclosure relates to a method of manufacturing a three-dimensional culture of a synapse comprising one or a plurality of spheroids in a culture vessel comprising a solid substrate, said method comprising:
(a) contacting one or a plurality of neuronal cells with the solid substrate, said substrate comprising at least one exterior surface, at least one interior surface and at least one interior chamber defined by the at least one interior surface and accessible from a point exterior to the solid substrate through at least one opening;
(b) positioning one or a plurality of spheroids comprising neuronal cells to the at least one interior chamber; and
(c) applying a cell medium into the culture vessel with a volume of cell medium sufficient to cover the one or plurality of neuronal cells; wherein at least one portion of the interior surface comprises a first cell-impenetrable polymer and a first cell-penetrable polymer.

In some embodiments, the solid substrate comprises a first a second interior chamber defined by the interior surface and wherein step (b) comprises positioning a spheroid comprising an isolated dorsal root ganglion in the first chamber and positioning a spinal cord explant in the second chamber. In some embodiments, the neuronal cells are cultured for a period of time sufficient to create a spheroid in the first and/or second chamber and wherein the neuronal cells in the spheroid are chosen from: motor neurons, sensory neurons, sympathetic neurons, parasympathetic neurons, cortical neurons, spinal cord neurons, peripheral neurons or combinations of any thereof. In some embodiments, the neuronal cells are human. In some embodiments, the neuronal cells are derived from stem cells, optionally, human stem cells.

The disclosure also relates to a method of screening one or a plurality of test agents for neuromodulatory effect, within a composition comprising: (i) a first spheroid of cells in electrical communication with a second spheroid of cells; (ii) a first electrode proximate to the first spheroid; and (iii) a second electrode proximate to the second spheroid, the method comprising:
(a) applying a voltage across the first and second electrodes at the first electrode for a sufficient time period and with a sufficient voltage to generate a detectable control waveform from the second electrode;
(b) detecting the control waveform from the second electrode.
   In some embodiments, the control waveform comprises a first and a second, fast negative-going wave followed by a third positive-going wave. In some embodiments, the method further comprises a step of detecting a waveform associated with the field potential of the synapse in the presence of the test agent. In some embodiments, the method further comprises a step of analyzing the waveform associated with a field potential in the presence of the test agent with the control waveform to determine neuromodulatory effect. In some embodiments, the step of analyzing comprises comparing the duration of the first, second, or third peaks of the waveform associated with a field potential in the presence of a test agent with the duration of the first, second, or third peaks of the control wave form; and, if the duration of the first second or third peaks of the waveform are increased in the presence of the test agent, the test agent is an analgesic. In some embodiments, the test agent is an opioid.

The present disclosure relates to a composition comprising a spheroid of cells comprising one or a combination of cells and/or tissues chosen from: a neuronal cell, nervous system ganglia, a stem cell, and an immune cell. In some embodiments, the spheroid comprises a tissue chosen from: a dorsal root ganglia and a trigeminal ganglia. In some embodiments, the spheroid comprises one or a plurality of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, astrocytes, oligodendrocytes, Schwann cells, microglia, ependymal cells, radial glia, satellite cells, enteric glial cells, and pituyicytes. In some embodiments, the spheroid comprises one or a plurality of glial cells. In some embodiments, the spheroid comprises one or a plurality of embryonic cells. In some embodiments, the spheroid comprises one or a plurality of mesenchymal stem cells. In some embodiments, the spheroid comprises one or a plurality of cells derived from an induced pluripotent stem cell. In some embodiments, the spheroid comprises one or a plurality of parasympathetic neurons. In some embodiments, the spheroid comprises one or a plurality of spinal motor neurons. In some embodiments, the spheroid comprises one or a plurality of central nervous system neurons. In some embodiments, the spheroid comprises one or a plurality of peripheral nervous system neurons. In some embodiments, the spheroid comprises one or a plurality of enteric nervous system neurons. In some embodiments, the spheroid comprises one or a plurality of motor neurons. In some embodiments, the spheroid comprises one or a plurality of sensory neurons. In some embodiments, the spheroid comprises one or a plurality of interneurons. In some embodiments, the spheroid comprises one or a plurality of cholinergic neurons. In some embodiments, the spheroid comprises one or a plurality of GABAergic neurons. In some embodiments, the spheroid comprises one or a plurality of glutamergic neurons, In some embodiments, the spheroid comprises one or a plurality of dopaminergic neurons. In some embodiments, the spheroid comprises one or a plurality of serotonergic neurons. In some embodiments, the spheroid comprises one or a plurality of trigeminal ganglion cells. In some embodiments, the spheroid comprises one or a plurality of astrocytes. In some embodiments, the spheroid comprises one or a plurality of oligodendrocytes. In some embodiments, the spheroid comprises one or a plurality of Schwann cells. In some embodiments, the spheroid comprises one or a plurality of microglial cells. In some embodiments, the spheroid comprises one or a plurality of ependymal cells. In some embodiments, the spheroid comprises one or a plurality of radial glia. In some embodiments, the spheroid comprises one or a plurality of satellite cells. In some embodiments, the spheroid comprises one or a plurality of enteric glial cells. In some embodiments, the spheroid comprises one or a plurality of pituyicytes.

In some embodiments, the spheroid comprises one or a plurality of one or combination of immune cells chosen from: a T cell, B cell, macrophage and astrocytes. In some embodiments, the spheroid comprises one or a plurality of one or a combination of stem cells chosen from: an embryonic stem cell, a mesenchymal stem cell, and an induced pluripotent stem cell. In some embodiments, the neuronal cell is derived from a stem cell chosen from: an embryonic stem cell, a mesenchymal stem cell, and an induced pluripotent stem cell. Embodiments include each of the above-mentioned cell types with each other individually or in combination.

In some embodiments, the spheroid has a diameter from about 200 microns to about 700 microns. In some embodiments, the spheroid has a diameter from about 150 microns to about 800 microns. In some embodiments, the spheroid has a diameter of about 200 microns. In some embodiments, the spheroid has a diameter of about 300 microns. In some embodiments, the spheroid has a diameter of about 400 microns. In some embodiments, the spheroid has a diameter of about 500 microns. In some embodiments, the spheroid has a diameter of about 600 microns. In some embodiments, the spheroid has a diameter of about 700 microns. In some embodiments, the spheroid has a diameter of about 800 microns. In some embodiments, the spheroid has a diameter of about 900 microns. In some embodiments, the spheroid has a diameter of about 350 microns. In some embodiments, the spheroid has a diameter of about 450 microns. In some embodiments, the spheroid has a diameter of about 550 microns. In some embodiments, the spheroid has a diameter of about 650 microns.

In some embodiments, the spheroid comprises one or a plurality of neuronal cells and one or a plurality of Schwann cells at a ratio of cell types equal to about 4 neuronal cells for every 1 Schwann cell. In some embodiments, the spheroid comprises one or a plurality of neuronal cells and one or a plurality of astrocytes at a ratio of about 4 neuronal cells for every 1 astrocyte. In some embodiments, the spheroid comprises one or a plurality of neuronal cells and one or a plurality of astrocytes at a ratio of about 1 neuronal cell for every 1 astrocyte. In some embodiments, the spheroid comprises one or a plurality of neuronal cells and one or a plurality of Schwann cells at a ratio of about 10 neuronal cells for every 1 Schwann cell. In some embodiments, the spheroid comprises one or a plurality of neuronal cells and one or a plurality of glial cells at a ratio equal to about four neuronal cells for every 1 glial cell.

In some embodiments, any one or plurality of cells described herein are differentiated from induced pluripotent stem cells. In some embodiments, the spheroid are free of induced pluripotent stem cells and/or immune cells. In some embodiments, the spheroid are free of undifferentiated stem cells.

In some embodiments, the spheroid comprises no less than about 20,000, 25,000, 26,000, 27,000, 28,000, 29,000, 30,000, 35,000, 40,000, 45,000, 50,000, 55,000, 60,000, 65,000, 70,000, or 75,000 cells. In some embodiments, the spheroid comprises no less than 75,000 cells. In some embodiments, the spheroid comprises no less than 65,000 cells. In some embodiments, the spheroid comprises no less than 60,000 cells. In some embodiments, the spheroid comprises no less than 100,000 cells. In some embodiments, the spheroid comprises no less than 125,000 cells. In some embodiments, the spheroid comprises no less than 150,000 cells. In some embodiments, the spheroid comprises no less than 175,000 cells. In some embodiments, the spheroid comprises no less than 200,000 cells. In some embodiments, the spheroid comprises no less than 225,000 cells. In some embodiments, the spheroid comprises no less than 250,000 cells. In some embodiments, the spheroid comprises no less than 12,500 cells. In some embodiments, the spheroid comprises from about 12,500 cells to about 250,000 cells. In some embodiments, the spheroid comprises from about 12,500 cells to about 100,000 cells. In some embodiments, the spheroid comprises from about 12,500 cells to about 75,000 cells.

In some embodiments, the spheroid further comprises one or a plurality of magnetic particles. In some embodiments the magnetic particles comprise one or more hollow interiors. In some embodiments, the magnetic particles comprises one or more layers of polymer onto which the cells form a spheroid.

The present disclosure also relates to a system comprising: (i) a cell culture vessel comprising a hydrogel; (ii) one or a plurality of spheroids comprising one or plurality of neuronal cells and/or isolated tissue explants; (iii) an amplifier comprising a generator for electrical current; (iv) a voltmeter and/or ammeter; and (v) at least a first stimulating electrode and at least a first recording electrode; wherein the amplifier, voltmeter and/or ammeter, and electrodes are electrically connected to the each other via a circuit in which electrical current is fed to the at least one stimulating electrode from the amplifier and electrical current is received at the recording electrode and fed to the voltmeter and/or ammeter; wherein the stimulating electrode is positioned at or proximate to one or a plurality of soma of the neuronal cells and/or isolated tissue explants and the recording electrode is positioned at a predetermined distance distal to the soma, such that an electrical field is established across the cell culture vessel. In some embodiments, the spheroid is any of the spheroids described herein.

In some embodiments, the culture vessel comprises 96, 192, 384 or more interior chambers. In some embodiments, the 96, 192, 384 or more interior chambers comprise one or plurality of isolated Schwann cells and/or one or plurality of oligodendrocytes sufficiently proximate to the one or plurality of isolated tissue explants and/or the one or plurality of neuronal cells such that the Schwann cells or the oligodendrocytes deposit myelin to axon growth from the tissue explants and/or neuronal cells.

In some embodiments, the system further comprises a solid substrate onto which the hydrogel matrix is crosslinked, said solid substrate comprising at least one plastic surface with pores from about 1 micron to about 5 microns in diameter. In some embodiments, the solid substrate comprises a contiguous exterior surface and an interior surface, such solid substrate comprising at least one portion in a cylindrical or substantially cylindrical shape and at least one hollow interior defined at its edge by at least one portion of the interior surface, said interior surface comprising one or a plurality of pores from about 0.1 microns to about 1.0 microns in diameter wherein the hollow interior of the solid substrate is accessible from a point exterior to the solid substrate through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with the hydrogel matrix, and wherein the second portion of the at least one hollow interior is in fluid communication with the first portion such that axons are capable of growth from the one or plurality of neuronal cells and/or the one or plurality of tissue explants into the second interior portion of the hollow interior.

In some embodiments, the system or composition is free of a sponge. In some embodiments, the hydrogel comprises at least a first cell-impenetrable polymer and a first cell-penetrable polymer. In some embodiments, the at least one cell-impenetrable polymer comprises no greater than about 15% PEG and the at least one cell-penetrable polymer comprises from about 0.05% to about 1.00% of one or a combination of self-assembling peptides chosen from: RAD 16-I, RAD 16-II, EAK 16-I, EAK 16-II, and dEAK 16. In some embodiments, the composition is free of polyethylene glycol (PEG). In some embodiments, the hydrogel comprises a first region and a second region, the first region is formed in the shape of a cylinder or rectangular prism oriented with its longitudinal axis passing through the top and bottom of the cell culture vessel and each of either the cylinder or rectangular prism comprising a space defined by an inner surface of the cylinder or rectangular prism, said space and accessible by one or more openings through the top of the cell culture vessel; wherein the second region comprises a space formed in the shape of its interior walls with an opening on its side adjacent to and in fluid communication with the first region. In some embodiments, the hydrogel comprises at least 1% polyethylene glycol (PEG). In some embodiments, the system further comprises a cell medium comprising nerve growth factor (NGF) at a concentration from about 5 to about 20 picograms per milliliter and/or ascorbic acid in a concentration ranging from about 0.001% weight by volume to about 0.01% weight by volume.

### BRIEF DESCRIPTION OF DRAWINGS

FIG. 1. Fabrication of embryonic rat DRG-SCDH microphysiological cocultures. (A) All DRG (green) and SCDH (red) nerve tissue is harvested from an entire litter of gestation day 15 rat embryos. (B) Tissue is pooled by type, dissociated into a single cell suspension, and aggregated in spheroid microplates to generate a batch of spheroids identical in size and composition. (C) A growth-restrictive outer-gel polyethylene glycol mold is fabricated to shape the cultures, spheroids are seeded in the mold, and the mold is filled with growth-permissive Matrigel. Over three weeks of culture, microphysiological tissue emerges from which system-level functional data are obtained.

FIG. 2. Live tissue imaging of virally expressed GFP in microphysiological DRG and SCDH spheroid cocultures. After 17 days in microphysiological culture, (A) DRG and (B) SCDH spheroid monocultures extend neurites the entire length of the hydrogel scaffold. In coculture, (C) DRG neurite growth is unaffected by the presence of SCDH spheroids while (D) SCDH neurite outgrowth is inhibited by the presence of the DRG spheroid, resulting in a unidirectional DRG to SCDH neurite growth pattern. In the main growth channel (˜0.5-1.5 mm), (E) there were no significant differences in GFP-expressing DRG neurite density between DRG monocultures and DRG-SCDH cocultures, while (F) GFP-expressing SCDH neurite density was significantly decreased in DRG-SCDH cocultures relative to SCDH monocultures. (G) A depth-coded three-dimensional rendering of virally encoded GFP expression indicates that the majority DRG tissue (left spheroid) adopts a relatively long, flat morphology less than 150 μm in height while SCDH tissue (right spheroid) maintains a relatively tall, spherical morphology with much of the spheroid near 250 μm in height. (H) Neuronal soma in the DRG spheroid region are relatively large and extend a single large axonal process easily distinguished under 20× magnification. In contrast, (I) SCDH neuronal soma are smaller and extend multiple small processes forming a mesh of dendritic and axonal processes. These results illustrate emergence of unidirectional DRG-SCDH neurite growth, nerve morphology, and neuronal morphology in microphysiological coculture that is consistent with in vivo DRG and SCDH tissue. *p<0.05, 2-way mixed model ANOVA with LSD post-hoc test.

FIG. 3. Punctate synaptic markers localize to SCDH but not to DRG spheroids in DRG-SCDH cocultures. Immunofluorescent labeling of the presynaptic marker, synapsin-I (Syn), the DRG-derived nerve fiber marker, calcitonin gene-related peptide (CGRP), and the dendritic cytoskeletal marker, microtubule-associated protein-2 (MAP2), is detectable in both spheroid regions. In composite images, (A) highly punctate synapsin-I immunoreactivity is detectable on and around MAP-2 positive somal and dendritic regions in the SCDH, consistent with synaptic nerve terminals, while (B) synapsin-I positive puncta are absent from the DRG spheroid, consistent with a lack of functional synapses. Individual channels comprising the SCDH (C-F) and DRG (G-J) composite images were subjected to identical thresholding and converted to binary masks for colocalization analysis. Colocalization of CGRP and synapsin-1 was observed in both the SCDH spheroid (K) and DRG spheroid (O) regions. However, CGRP/synapsin-1 colocalization was punctate in the SCDH (L) but expressed diffusely throughout the cell somas of the DRG spheroid (P). Furthermore, 39% of CGRP/synapsin-1 puncta also tri-colocalized with MAP2 expression in the SCDH spheroid (M), consistent with a post-synaptic connection, while only 2% colocalized with DAPI (N), which is more consistent with somal expression. In contrast, only 19% of CGRP/synapsin-1 puncta colocalize with MAP2 expression in DRG spheroids while 35% colocalize with DAPI. Together these data indicate a greater potential for functional synapse formation in the SCDH, including both afferent, synapsin-I+/CGRP+, DRG-SCDH synapses and recurrent, synapsin-I+/CGRP−, SCDH-SCDH synapses, than in the DRG which preferentially displays intracellular expression of synaptic proteins.

FIG. 4. Optogenetic stimulation of DRG tissue excites SCDH nerve tissue through glutamatergic neurotransmission in microphysiological coculture. AAV-dependent expression of a channelrhodopsin-green fluorescent protein (CHR2-GFP) fusion protein renders microphysiological nerve tissue optically excitable by 488 nm light and AAV-dependent expression of CaMPARI-2 renders calcium currents optically recordable through calcium-dependent green-to-red photoconversion during concurrent exposure to 405 nm light. (A) Red CaMPARI-2 fluorescence was measured at baseline (top) and after photostimulation (bottom) to quantify CHR2-induced (left), baseline (middle), and capsaicin-induced (right) calcium currents in DRG monocultures. CaMPARI-2 recorded calcium currents are significantly greater than baseline calcium currents during (B) activation of CHR2-GFP and (C) capsaicin treatment. (B,C) CHR2-induced calcium currents are larger than capsaicin-induced calcium currents confirming CHR2-induced nerve excitation is large enough for physiological relevance. (D) Baseline green (top) and red (middle) fluorescence recorded from coculture of a wild-type DRG spheroid (left) or a DRG spheroid virally expressing CHR2-GFP (right) with a CaMPARI-2 expressing SCDH spheroid. Following photostimulation, a baseline amount of green-to-red CaMPARI-2 photoconversion is detectable in DRG(WT)-SCDH(CaMPARI-2) constructs (bottom, left), while a larger amount of CaMPARI-2 photoconversion is detectable in DRG(CHR2-GFP)-SCDH(CaMPARI-2) constructs (bottom, right). (E) Photostimulation of CHR2-GFP in DRG tissue significantly increases calcium influx in SCDH relative to DRG(WT) controls. (F) Baseline and post-photostimulation red fluorescence observed in DRG(CHR2-GFP)-SCDH(CaMPARI-2) after pretreatment with the AMPA/Kainate receptor antagonist CNQX (right) or vehicle alone (left). (G) Inhibition of glutamatergic neurotransmission with CNQX significantly reduces DRG(CHR2-GFP) photostimulation-induced calcium influx in SCDH. Data presented as mean+/−SEM. *p<0.05 by an unpaired t-test.

FIG. 5. A distinct, replicable, three-part waveform emerges in the SCDH spheroid region of DRG-SCDH cocultures. (A) Only a single, fast, negative-going field potential, denoted N1, is recorded in the nerve region of DRG monocultures upon stimulation of the DRG spheroid region. (B) Concerted field potentials were not recorded in the spheroid region of SCDH monocultures upon stimulation of the nerve region. When DRG and SCDH spheroids are cocultured, (C) a three-part waveform is recorded in the SCDH spheroid region upon stimulation of the DRG spheroid region. This waveform is comprised of two fast, negative-going field potentials, denoted N1 and N2, and a slower, prolonged, positive-going field potential, denoted P1. N1 is likely a recording of the concerted depolarization of DRG nerve fibers running through the SCDH spheroid region that are observed in the absence of the SCDH spheroid as shown in panel A. N2 and P1 are novel field potentials that are not observed in the absence of the DRG spheroid and are therefore potentially a result of microphysiological synaptic transmission between DRG and SCDH nerve tissue.

FIG. 6. Synaptic waveform components N2 and P1, but not electrically-evoked N1, fatigue under repeated stimulation. DRG spheroids were repeatedly stimulated for one second at increasing frequencies of 10 Hz, 20 Hz, and 25 Hz while continuously recording field potential production in the SCDH of DRG-SCDH cocultures. (A) Comparison of the overall time course of the 10 Hz (black) and 25 Hz (red) experiments for a representative construct. (B) The amplitude of the N1, N2, and P1 waveform components was measured after each stimulation and normalized to initial amplitudes to calculate the percent change in each component relative to baseline. N1 amplitude remains unchanged across the first ten stimulations at each stimulation frequency. A significant reduction in N2 amplitude is observed after the tenth stimulation at 25 Hz and a significant reduction in P1 amplitude is observed after the tenth stimulation at frequencies of 10 Hz and above. (C) Baseline waveforms recorded after the first stimulation at 10 Hz (black) and 25 Hz (red) are similar with clearly identifiable N1, N2, and P1 peaks. (D) N2 and P1 are absent after the tenth stimulation at 25 Hz (red) and P1 is still present but significantly reduced after the tenth stimulation at 10 Hz (black). Data presented as mean+/−SEM. #p<0.05, one sample t-test vs 0%.

FIG. 7. Both glutamatergic and GABAergic neurotransmission shape the DRG-SCDH microphysiological synaptic waveform. DRG nerve tissue was electrically stimulated and resulting field potentials were recorded in the SCDH of DRG-SCDH cocultures in the presence of (A) the calcium chelator, EDTA, (B) the AMPA/Kainate receptor antagonist, CNQX, or (C) the GABA_{A}-receptor antagonist, bicuculline. Inhibition of all synaptic transmission with EDTA (D) or inhibition of glutamatergic AMPA/Kainate receptor signaling with CNQX (E) significantly reduces the amplitude of the N2 and P1 peaks while having no effect on N1. (F) Inhibition of GABA_{A}-receptor signaling with bicuculline specifically enhances the late phase of the P1 peak while having no effect on N2 or N1. Amp, amplitude. Lat, latency. AUC, area under curve. Data presented as mean+/−SEM. #p<0.05, one sample t-test vs 0%.

FIG. 8. The commonly used analgesics lidocaine, clonidine, and morphine differentially modulate synaptic transmission in a microphysiological model of lower afferent pain signaling. Differential desensitization of the afferent DRG input was controlled for by matching the maximal post-treatment to its pre-treatment trace with the most similar N1 amplitude. Example N1-matched lidocaine (A), clonidine (B), and morphine (C) traces. Statistical analysis of N1-matched traces indicated that (D) all analgesics significantly increased the latency to both N1 and N2 peaks, (E) clonidine more specifically reduced the maximum amplitude of P1 while morphine significantly reduced the amplitude of N2, and (F) clonidine significantly attenuated the early phase of P1 while morphine significantly increased the late phase of P1. N1, N2, and P1 amplitude matching was performed and the difference in stimulus intensity required to produce an N1, N2, and P1 peak of equal amplitude before and after analgesic treatment was calculated to quantify differential desensitization of each of the peak components. (G) Statistical analysis of the change in required stimulus intensity indicated that lidocaine primarily desensitized N1 while clonidine and morphine primarily desensitize N2 and P1. Data presented as mean+/−SEM. #p<0.05 one sample t-test vs 0 (no change).

FIG. 9. Viscoelastic evaluation of different gelatin methacrylate and Matrigel formulations. (A) Storage modulus (G′) and (B) loss modulus (G″) across 0-10 Hz frequencies determined through rheological characterization of gels. (C) Quantification of storage modulus for all gels measured at 10 Hz confirms the increasing gel stiffness across different formulations of Matrigel and gelatin methacrylate. Unless otherwise indicated, statistical significance between groups is very highly significant (****, p<0.0001). n=3. NS=no significance, **=p<0.01 and ***=p<0.001. (D) Visualization of spheroid and viability in Matrigel and gelatin methacrylate formulations of varying stiffness at DIV 10 using calcein AM confirms comparable viability across all gel formulations. Scale bar=250 μM. (E) Representative maximum intensity projection images of neurite outgrowth area at DIV 10 after whole-construct immunofluorescent staining of the neuron-specific cytoskeletal protein beta-III-tubulin indicates impaired SCDH growth in stiffer, gelatin methacrylate-based, gel formulations. DRG nerve growth was comparably robust across all gel formulations. Scale bar=200 μM. (F) Comparison of DRG and DH neurite outgrowth in gelatin methacrylate formulations at DIV 10 with calcein AM viability stain illustrates robust growth of DRG tissue towards SCDH spheroids over time. Scale bar=200 μM.

FIG. 10. DRG and SCDH spheroid cultures retain peripheral and central nerve phenotypes after three weeks in microphysiological culture. (A) Schematic of construct orientation with DRG spheroids at the top and SCDH spheroids at the bottom of the image 2 mm apart. (B) DAPI staining (dark grey) shows the presence of cells throughout the culture system. (C) Beta-II-tubulin immunostaining (light grey) confirms that all tissue maintains a neural phenotype in culture and marks the extent of neurite outgrowth. (D) Peripherin immunostaining (green) confirms that DRG-derived neural tissue retains a peripheral nerve phenotype. (E) Merged image confirms colocalization of peripherin and beta-III-tubulin in DRG but not SCDH spheroid region. Synaptic markers colocalize in spinal cord dorsal horn. Synapse-related proteins are highly expressed in the SCDH but not DRG spheroid regions of DRG-SCDH cocultures. (F) The presynaptic marker, synapsin-I, and (G) the post-synaptic maker, post-synaptic density protein 95 (PSD95), (H) merged image of synapsin-I and PSD95, (I) the dendritic marker, microtubule associated protein 2, (MAP2), and (J) the GABAergic nerve terminal marker, vesicular GABA transporter (vGAT), are all expressed more strongly in the SCDH than DRG spheroid. This suggests that there is greater potential for formation of functional synapses in the SCDH than DRG spheroid region.

FIG. 11. Extracellular field potential production was qualitatively evaluated throughout DRG and SCDH monocultures, DRG-DRG cocultures, SCDH-SCDH cocultures, and DRG-SCDH cocultures. (A) Field potentials recorded in the spheroid region after stimulation of nerve outgrowth consist of a large negative-going population spike followed by several smaller negative-going peaks while (B) field potentials recorded in the nerve outgrowth after stimulation of the spheroid region consists of a single similar large negative-going population spike lacking subsequent smaller peaks. (D) In DRG-DRG cocultures, stimulation of one DRG spheroid coculture results in multiple, large negative going spikes in the adjacent spheroid. (D,E) Electrically-evoked CAP conduction was not detectable in the nerve or spheroid region of SCDH spheroid monocultures after direct stimulation. (F) In SCDH-SCDH cocultures, stimulation of one spheroid produced small, asynchronous negative going spikes followed by a slower, positive-going wave, though these recordings lacked consistency (data not shown). (G) In DRG-SCDH cocultures, a consistent and unique waveform indicative of synaptic transmission emerges in SCDH spheroids after stimulation of DRG spheroids that is distinct from waveforms observed in DRG or SCDH monocultures, DRG-DRG cocultures, or SCDH-SCDH cocultures. This waveform consists of two, fast negative-going population spikes (denoted N1 and N2) followed by a slower but prolonged positive-going wave (denoted P1, FIG. S3G). (H) Waveforms recorded in the DRG spheroid region of DRG-SCDH cocultures are nearly identical to waveforms recorded in DRG monocultures indicating that DRG spheroid electrophysiology is unaffected by the presence of the SCDH spheroid. (I) Stimulation of the distal DRG neurite growth confirms that this synaptic waveform is not conflated with electrically-evoked SDCH spheroid activation resulting from direct stimulation of distal SCDH neurite growth. This distal stimulation site is well beyond the range of SCDH neurite outgrowth observed in DRG-SCDH coculture (FIG. 2D) but still produces a SCDH spheroid waveform containing the same three components as more proximal stimulation sites, albeit with the longer latency and reduced amplitude expected from a more distal stimulation.

FIG. 12. The N1 peak represents a direct recording of electrically-evoked concerted depolarization of the afferent, DRG-derived nerve fibers within the SCDH. (A) When recording in the nerve fiber region of a DRG monoculture, (C) a large, concerted, negative-going peak (N1) is recorded within the first 10 msec following stimulation of the DRG spheroid region. This peak represents the concerted depolarization of DRG nerve fibers in the region. Consistent with an electrically evoked potential, this peak is insensitive to (C) inhibition of glutamatergic neurotransmission with CNQX and (E) inhibition of synaptic transmission with EDTA but (G) is completely blocked after inhibition of voltage-gated sodium channels with tetrodotoxin (TTX). (B) When stimulating the DRG spheroid and recording in the SCDH spheroid of DRG-SCDH cocultures, (D) a similar large, concerted, negative-going peak (N1) is observed within the first 10 msec following stimulation of the DRG spheroid region but is now followed by the synaptic waveform components N2 and P1. In DRG-SCDH cocultures, the N1 peak is again insensitive to (D) CNQX and (F) EDTA but is (H) completely blocked by TTX. Therefore, we conclude that the N1 peak in of the synaptic waveform represents electrically evoked presynaptic fiber volley.

FIG. 13: Identification of lowest effective dose of lidocaine, clonidine, and morphine in microphysiological DRG-SCDH coculture. The dose of lidocaine progressively increased from (A) 10 μM, (B) 100 μM, (C) 500 μM, and (D) 1 mM. Increased latencies of N1 and N2 first became apparent after treatment with 100 μM lidocaine, became more severe at 500 μM, and electrical activity was completely inhibited at the 1 mM dose. The dose of clonidine progressively increased from (E) 100 nM, (F) 1 μM, and (G) 10 μM. Increased latencies of N1 and N2 and reshaping of P1 are first evident after treatment with 1 uM clonidine and electrical activity was completely inhibited at the 10 uM dose. The dose of morphine progressively increased from (H) 1 μM, (I) 10 μM, (J) 100 μM, and (K) 500 μM. Reshaping of P1 is first evident after treatment with 100 μM morphine while increased latency to N1 and N2 become apparent at 500 μM.

FIG. 14. Analgesic effects on microphysiological DRG-SCDH coculture synaptic physiology are reversible by washout. (A) Constructs treated with lidocaine display increased latencies after treatment with 100 uM lidocaine and (B) a complete blockade of electrical activity at 1 mM. (C) Electrical activity is again detectable in the same construct after a 60-minute washout period. (D) Re-application of 1 mM lidocaine is again effective following the washout period, confirming the reversibility of lidocaine's effect. Similarly, treatment with (E) 1 uM and (F) 10 uM clonidine progressively impairs synaptic transmission in DRG-SCDH cocultures. (G) Electrical activity is again detectable in the same construct after a 60-minute washout. Re-application of (H) 1 uM and (I) 10 uM clonidine is again effective after the washout, confirming the reversibility of clonidine's effects on synaptic transmission. (J) Treatment with 500 uM morphine increases N1 and N2 peak latencies and increases the late phase of the PL. (K) A 60-minute washout period reverses the effects of morphine, decreasing the latencies to N1 and N2 and decreasing the late phase of P1. (L) Re-application of 500 uM morphine again increases the latencies to N1 and N2 and increases the late phase of P1 confirming that the effects of morphine on microphysiological DRG-SCDH synaptic transmission are reversible.

FIG. 15: Unbiased analysis of analgesic-induced changes in N1, N2, and P1 latency and amplitude, as well as early, late, and total integrated area under the curve (AUC) of P1 with increasing stimulus intensity from 0 to 40V. The change in (A) N1 amplitude, (B) N2 amplitude, and (C) P1 amplitude between baseline and sham recordings (blue) and between sham recordings and post-analgesic treatment recordings (red, green, purple) at increasing stimulation intensities. (D) Graphical summary of one sample t-tests indicating significant or nearly significant changes in N1, N2, and P1 latencies (bottom) and amplitudes (middle) as well as early, late, and total integrated AUC of P1 (top) after stimulations of increasing intensity during sham treatment (left) and followed by treatment with lidocaine (middle left), clonidine (middle right) or morphine (right). (E) Color coded key matching each cell to the associated p-value. Arrangement of results of statistical analysis in the heatmap format aides in identification of corroboration across multiple related but distinct metrics and across increasing stimulation intensity. Sham treatment most consistently increased the amplitude of N2 and decreased the amplitude and AUC of P1. Lidocaine most specifically and significantly increased the latency, and decreased the amplitude, of N1 and N2 while having limited effects on the latency, amplitude, or shape of P1. Clonidine more sporadically increased the latencies of N1 and N2 and specifically decreased the early phase of P1. Morphine increased the latency to P1 at moderate stimulation intensities, consistently decreased the amplitude of N2, and consistently increase the late and total AUC of P1.

FIG. 16: The electrophysiological signature of hPSC-derived cocultures of nociceptor and dorsal spinal cord spheroids is shaped by functional excitatory, glutamatergic and inhibitory, GABAergic synaptic connections after 6-8 weeks of maturation. (A) Sham treatment has minimal effect on the shape of the waveform observed in the dorsal spinal cord spheroid after electrical stimulation of the afferent nociceptor spheroid. (B) Application of the GABAa receptor antagonist, bicuculline (BCC), selectively enhances the late, positive-going portion of the synaptic wave form while (C) application of the AMPA-type glutamatergic receptor antagonist, CNQX, blocks a large portion of the synaptic waveform. (D) Sham treatment had minimal effect on the waveform across multiple trials. (E) The largest effect of bicuculline application is an increase in the area under the curve (AUC) of the late P1 portion of the synaptic waveform. (F) CNQX application signification reduced the amplitude of the later negative and positive-going portions of the synaptic waveform.

FIG. 17: Opioid receptor activation selectively prolongs the late, positive-going portion of the synaptic waveform in hPSC-derived nociceptor-dorsal spinal cord cocultures. (A) The late, positive-going wave is enhanced after treatment with 100 uM morphine relative to sham-treated cultures. (B) This effect was consistent across multiple cultures.

FIG. 18: Emergence of semi-spontaneous and fully spontaneous circuit in embryonic rat DRG-SCDH and hPSC-derived nociceptor-dorsal spinal cord cocultures with prolonged maturation. (A) After 3 months of maturation, repetitive semi-spontaneous burst firing is observed in the SCDH spheroid of embryonic rat DRG-SCDH cocultures several seconds after stimulation of afferent DRG tissue. (B) Semi-spontaneous burst firing in the same culture is increased in both amplitude and frequency following treatment with 100 uM morphine. (C) After 3 months of maturation, similar repetitive semi-spontaneous burst firing is observed in the SCDH spheroid of hPSC-derived nociceptor-SCDH cocultures several seconds after stimulation of afferent nociceptor tissue. (D) Semi-spontaneous burst firing in the same tissue is increased in frequency after application of 100 uM morphine. (E) After 6 months of maturation, fully spontaneous burst firing is observed in the SCDH spheroid of hPSC-derived nociceptor-SCDH cocultures several seconds in absence of any afferent stimulation. (F) Spontaneous burst firing increases in frequency and amplitude after application of 100 uM morphine.

FIG. 19: Long-term depression in young hPSC-derived nociceptor-dorsal spinal cord microphysiological nerve cultures. (A) Distinct early negative-going peaks and a later, positive-going wave of activity are clearly detectable following the first 40 V stimulation experienced by the nerve construct. (B) The response to the same 40 V stimulus following three consecutive stimulus response curve comprised of ten, 0.5 hz stimulations at each of 0, 1, 2, 3, 4, 5, 6, 7, 8, 9, 10, 15, 20, 30, and 40 V is much reduced in amplitude and complexity. The negative going activity is much smaller in amplitude and the later positive-going wave of activity is nearly undetectable.

### DETAILED DESCRIPTION OF EMBODIMENTS

Various terms relating to the methods and other aspects of the present disclosure are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “more than 2” as used herein is defined as any whole integer greater than the number two, e.g. 3, 4, or 5.

The term “about” as used herein when referring to a measurable value such as an amount, a temporal duration, and the like, is meant to encompass variations of 20%, ±10%, 5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law. As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

The term “plurality” as used herein is defined as any amount or number greater or more than 1.

The term “vessel” as used herein is defined as any vessel suitable for growing, culturing, cultivating, proliferating, propagating, or otherwise similarly manipulating cells. A culture vessel may also be referred to herein as a “culture insert”. In some embodiments, the culture vessel is designed to comprise an interior chamber into which the disclosed tissue is positioned and various culture mediums.

Wherever any of the phrases “for example,” “such as,” “including” and the like are used herein, the phrase “and without limitation” is understood to follow unless explicitly stated otherwise. Similarly, “an example,” “exemplary” and the like are understood to be non-limiting.

The term “substantially” allows for deviations from the descriptor that do not negatively impact the intended purpose. In some embodiments, the term “substantially” in connection with a distance from one item relative to a second item element, means that the first item is from about 1 micron to about 100 microns in distance to the second item.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context. In some embodiments, the

The term “seeding” as used herein is defined as transferring an amount of cells or tissue into a culture vessel. The amount may be defined and may use volume or number of cells as the basis of the defined amount. The cells may be part of a suspension or printed onto the three-dimensional culture. In some embodiments, cells utilized in accordance with the present disclosure are cells that retain viability, and optionally growth capabilities, when in the form of a three-dimensional culture. In some embodiments, cells are eukaryotic cells. In some embodiments, cells are human cells. In some embodiments, cells are rodent cells. In some embodiments, the cells are mouse cells. In some embodiments, the cells are a mixture of human and rodent cells. In some embodiments, cells are obtained from transformed cells in culture. In some embodiments, cells are obtained from a living organism. In some embodiments, cells comprise neuronal cells. In some embodiments, the cells comprise immune cells. In some embodiments, cells are blood cells. In some embodiments, cells comprise nerve cells. In some embodiments, the cells comprise epithelial cells. In some embodiments, cells comprise stem cells. In some embodiments, the cells comprise human cells. In some embodiments, the cells are human cells. In some embodiments, the cells comprise cells derived from induced pluripotent stem cells.

The term “culture vessel” as used herein can be any vessel suitable for growing, culturing, cultivating, proliferating, propagating, or otherwise similarly manipulating cells. A culture vessel may also be referred to herein as a “culture insert”. In some embodiments, the culture vessel is made out of biocompatible plastic and/or glass. In some embodiments, the plastic is a thin layer of plastic comprising one or a plurality of pores that allow diffusion of protein, nucleic acid, nutrients (such as heavy metals and hormones) antibiotics, and other cell culture medium components through the pores. In some embodiments, the pores are not more than about 0.1, 0.5 1.0, 2, 3, 4, 5, 6, 7, 8, 9, 10, 15, 20, 25, 30, 40, 50 microns wide. In some embodiments, the culture vessel in a hydrogel matrix and free of a base or any other structure. In some embodiments, the culture vessel is designed to contain a hydrogel or hydrogel matrix and various culture mediums. In some embodiments, the culture vessel consists of or consists essentially of a hydrogel or hydrogel matrix. In some embodiments, the only plastic component of the culture vessel is the components of the culture vessel that make up the side walls and/or bottom of the culture vessel that separate the volume of a well or zone of cellular growth from a point exterior to the culture vessel. In some embodiments, the culture vessel comprises a hydrogel and one or a plurality of isolated glial cells. In some embodiments, the culture vessel comprises a hydrogel and one or a plurality of isolated glial cells, to which one or a plurality of neuronal cells are seeded.

The term “electrical stimulation” refers to a process in which the cells are being exposed to an electrical current of either alternating current (AC) or direct current (DC). The current may be introduced into the solid substrate or applied via the cell culture media or other suitable components of the cell culture system. In some embodiments, the electrical stimulation is provided to the device or system by positioning one or a plurality of electrodes at different positions within the device or system to create a voltage potential across the cell culture vessel. The electrodes are in operable connection with one or a plurality of amplifiers, voltmeters, ammeters, and/or electrochemical systems (such as batteries or electrical generators) by one or a plurality of wires. Such devices and wires create a circuit through which an electrical current is produced and by which an electrical potential is produced across the tissue culture system.

The term “hydrogel” as used herein can be, for example, any water-insoluble, crosslinked, three-dimensional network of polymer chains with the voids between polymer chains filled with or capable of being filled with water. The term “hydrogel matrix” as used herein refers to, for example, any three-dimensional hydrogel construct, system, device, or similar structure. Hydrogels and hydrogel matrices are known in the art and various types have been described, for example, in U.S. Pat. Nos. 5,700,289, and 6,129,761; and in Curley and Moore, 2011; Curley et al., 2011; Irons et al., 2008; and Tibbitt and Anseth, 2009; each of which are incorporated by reference in their entireties. In some embodiments, the hydrogel or hydrogel matrix can be solidified by subjecting the liquefied pregel solution to ultraviolet light, visible light or ay light above about 300 nm, 400 nm, 450 nm or 500 nm in wavelength. In some embodiments, the hydrogel or hydrogel matrix can be solidified into various shapes, for example, a bifurcating shape designed to mimic a neuronal tract. In some embodiments, the hydrogel or hydrogel matrix comprises poly (ethylene glycol) dimethacrylate (PEG). In some embodiments, the hydrogel or hydrogel matrix comprises Puramatrix. In some embodiments, the hydrogel or hydrogel matrix comprises glycidyl methacrylate-dextran (MeDex). In some embodiments, neuronal cells are incorporated in the hydrogel or hydrogel matrices. In some embodiments, cells from nervous system are incorporated into the hydrogel or hydrogel matrices. In some embodiments, the cells from nervous system are Schwann cells and/or oligodendrocytes. In some embodiments, the hydrogel or hydrogel matrix comprises tissue explants from the nervous system of an animal, (such as a mammal) and a supplemental population of cells derived from the nervous system but isolated and cultured to enrich its population in the culture. In some embodiments, the hydrogel or hydrogel matrix comprises a tissue explant such as a retinal tissue explant, DRG, or spinal cord tissue explant and a population of isolated and cultured Schwann cells, oligodendrocytes, and/or microglial cells. In some embodiments, two or more hydrogels or hydrogel matrixes are used simultaneously cell culture vessel. In some embodiments, two or more hydrogels or hydrogel matrixes are used simultaneously in the same cell culture vessel but the hydrogels are separated by a wall that create independently addressable microenvironments in the tissue culture vessel such as wells. In a multiplexed tissue culture vessel it is possible for some embodiments to include any number of aforementioned wells or independently addressable location within the cell culture vessel such that a hydrogel matrix in one well or location is different or the same as the hydrogel matrix in another well or location of the cell culture vessel.

The term “immune cell” as used herein can be any cell, for example, that participates in the immune activity of as subject, including defending a subject from infection or the symptom of infection or attacking, clearing or otherwise eliminating a dysfunction cell or pathogen from a cell in a subject, or improving the a symptoms of a disease caused by a pathogen. In some embodiments, immune cells comprise one or a plurality of B cells, T cells, antigen presenting cells such as astrocytes, dendritic cells and macrophages, stellate cells, granulocytes, monocytes, basophils, eosinophils, and/or mast cells. In some embodiments, the immune cell expresses CD4 or CD8 and one or more immunomodulatory molecule. In some embodiments, the immunomodulatory molecule is chosen from one of the following: IL-28, MHC, CD80, CD86, IL-1, IL-2, IL-4, IL-5, IL-6, IL-10, IL-18, MCP-1, MIP-Ia, MIP-I(3, IL-8, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, 1 kB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof, or a combination thereof. Immunomodulatory proteins are exemplified in U.S. Pat. No. 8,008,265.

The term “immunomodulatory” refers to a substance that has a modulatory effect on the immune system. Such substances can be readily identified using standard assays which indicate various aspects of the immune response, such as cytokine secretion, antibody production, NK cell activation and T cell proliferation. See, e.g., WO 97/28259; WO 98/16247; WO 99/11275; Krieg et al. (1995) Nature 374:546-549; Yamamoto et al. (1992) J. Immunol. 148:4072-76; Ballas et al. (1996) J. Immunol. 157:1840-45; Klinman et al. (1997) J. Immunol. 158:3635-39; Sato et al. (1996) Science 273:352-354; Pisetsky (1996) J. Immunol. 156:421-423; Shimada et al. (1986) Jpn. J. Cancer Res. 77:808816; Cowdery et al. (1996) J. Immunol. 156:4570-75; Roman et al. (1997) Nat. Med. 3:849-854; Lipford et al. (1997a) Eur. J. Immunol. 27:2340-44; WO 98/55495 and WO 00/61151. Accordingly, these and other methods can be used to identify, test and/or confirm immuno stimulatory substances, such as immuno stimulatory nucleotides, immunostimulatory isolated nucleic acids.

In some embodiments, the two or more hydrogels may comprise different amount of PEG and/or Puramatrix. In some embodiments, the two or more hydrogels may have various densities. In some embodiments, the two or more hydrogels may have various permeabilities that are capable of allowing cells to grow within the hydrogel. In some embodiments, the two or more hydrogels may have various flexibilitics. In some embodiments, the bioreactor, cell culture device or composition disclosed herein comprises a hydrogel comprising two layers of polymers: a cell-penetrable polymer and a cell-impenetrable polymer. In some embodiments, the cell-penetrable layer is layered at least in one region on top of the cell-impenetrable layer.

The term “cell-penetrable polymer” refers to a hydrophilic polymer, with identical or mixed monomer subunits, at a concentration and/or density sufficient to create spaces upon crosslinking in a solid or semisolid state on a solid substrate, such space are sufficiently biocompatible such that a cell or part of a cell can grow in culture.

The term “cell-impenetrable polymer” refers to a hydrophilic polymer, with identical or mixed monomer subunits, at a concentration and/or density sufficient to, upon crosslinking in a solid or semisolid state on a solid substrate, not create biocompatible spaces or compartments. In other words, an cell-impenetrable polymer is a polymer that, after crosslinking at a particular concentration and/or density, cannot support growth of a cell or part of a cell in culture.

The term “functional fragment” can be any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain 99%, 98%, 97%, 96%, 95%, 94%, 93%, 92%, 91%, or 90% sequence identity to the wild-type human sequence upon which the sequence is derived. In some embodiments, the functional fragment may retain 87%, 85%, 80%, 75%, 70%, 65%, or 60% sequence homology to the wild-type sequence upon which the sequence is derived.

One of ordinary skill can appreciate that a cell-impenetrable polymer and a cell-penetrable polymer may comprise the same or substantially the same polymers but the difference in concentration or density after crosslinking creates a hydrogel matrix with some portions conducive to grow a cell or part of cell in culture.

In some embodiments, the hydrogel or hydrogel matrixes can have various thicknesses. In some embodiments, the thickness of the hydrogel or hydrogel matrix is from about 100 pm to about 800 p.m. In some embodiments, the thickness of the hydrogel or hydrogel matrix is from about 150 vim to about 800 vim. In some embodiments, the thickness of the hydrogel or hydrogel matrix is from about 200 pm to about 800 vim. In some embodiments, the thickness of the hydrogel or hydrogel matrix is from about 250 vim to about 800 pm. In some embodiments, the thickness of the hydrogel or hydrogel matrix is from about 300 pm to about 800 pm. In some embodiments, the thickness of the hydrogel or hydrogel matrix is from about 350 pm to about 800 pm. In some embodiments, the thickness of the hydrogel or hydrogel matrix is from about 400 vim to about 800 vim. In some embodiments, the thickness of the hydrogel or hydrogel matrix is from about 450 pm to about 800 pm. In some embodiments, the thickness of the hydrogel or hydrogel matrix is from about 500 pm to about 800 μm. In some embodiments, the thickness of the hydrogel or hydrogel matrix is from about 550 vim to about 800 vim. In some embodiments, the thickness of the hydrogel or hydrogel matrix is from about 600 pm to about 800 vim. In some embodiments, the thickness of the hydrogel or hydrogel matrix is from about 650 vim to about 800 pm. In some embodiments, the thickness of the hydrogel or hydrogel matrix is from about 700 pm to about 800 pm. In some embodiments, the thickness of the hydrogel or hydrogel matrix is from about 750 vim to about 800 vim. In some embodiments, the thickness of the hydrogel or hydrogel matrix is from about 100 pm to about 750 vim. In some embodiments, the thickness of the hydrogel or hydrogel matrix is from about 100 vim to about 700 pm. In some embodiments, the thickness of the hydrogel or hydrogel matrix is from about 100 pm to about 650 pm. In some embodiments, the thickness of the hydrogel or hydrogel matrix is from about 100 vim to about 600 vim. In some embodiments, the thickness f the hydrogel or hydrogel matrix is from about 100 pm to about 550 pm. In some embodiments, the thickness of the hydrogel or hydrogel matrix is from about 100 pm to about 500 pm. In some embodiments, the thickness of the hydrogel or hydrogel matrix is from about 100 pm to about 450 pm. In some embodiments, the thickness of the hydrogel or hydrogel matrix is from about 100 μm to about 400 μm. In some embodiments, the thickness of the hydrogel or hydrogel matrix is from about 100 pm to about 350 μm. In some embodiments, the thickness of the hydrogel or hydrogel matrix is from about 100 μm to about 300 pm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 μm to about 150 μm. In some embodiments, the thickness of the hydrogel or hydrogel matrix is from about 300 pm to about 600 pm. In some embodiments, the thickness of the hydrogel or hydrogel matrix is from about 400 μm to about 500 μm.

In some embodiments, the hydrogel or hydrogel matrixes can have various thicknesses. In some embodiments, the thickness of the hydrogel or hydrogel matrix is from about 10 gm to about 3000 μm. In some embodiments, the thickness of the hydrogel or hydrogel matrix is from about 150 pm to about 3000 pm. In some embodiments, the thickness of the hydrogel or hydrogel matrix is from about 200 pm to about 3000 pm. In some embodiments, the thickness of the hydrogel or hydrogel matrix is from about 250 μm to about 3000 pm. In some embodiments, the thickness of the hydrogel or hydrogel matrix is from about 300 μm to about 3000 pm. In some embodiments, the thickness of the hydrogel or hydrogel matrix is from about 350 pm to about 3000 pm. In some embodiments, the thickness of the hydrogel or hydrogel matrix is from about 400 pm to about 3000 gm. In some embodiments, the thickness of the hydrogel or hydrogel matrix is from about 450 pm to about 3000 pm. In some embodiments, the thickness of the hydrogel or hydrogel matrix is from about 500 pm to about 3000 pm. In some embodiments, the thickness of the hydrogel or hydrogel matrix is from about 550 pm to about 3000 pm. In some embodiments, the thickness of the hydrogel or hydrogel matrix is from about 600 μm to about 3000 pm. In some embodiments, the thickness of the hydrogel or hydrogel matrix is from about 650 pm to about 3000 μm. In some embodiments, the thickness of the hydrogel or hydrogel matrix is from about 700 μm to about 3000 pm. In some embodiments, the thickness of the hydrogel or hydrogel matrix is from about 750 pm to about 3000 pm. In some embodiments, the thickness of the hydrogel or hydrogel matrix is from about 800 pm to about 3000 gm. In some embodiments, the thickness of the hydrogel or hydrogel matrix is from about 850 pm to about 3000 pm. In some embodiments, the thickness of the hydrogel or hydrogel matrix is from about 900 pm to about 3000 pm. In some embodiments, the thickness of the hydrogel or hydrogel matrix is from about 950 pm to about 3000 pm. In some embodiments, the thickness of the hydrogel or hydrogel matrix is from about 1000 pm to about 3000 μm. In some embodiments, the thickness of the hydrogel or hydrogel matrix is from about 1500 μm to about 3000 μm. In some embodiments, the thickness of the hydrogel or hydrogel matrix is from about 2000 μm to about 3000 gm. In some embodiments, the thickness of the hydrogel or hydrogel matrix is from about 2500 idtrl to about 3000 μm. In some embodiments, the thickness of the hydrogel or hydrogel matrix is from about 100 pm to about 2500 pm. In some embodiments, the thickness of the hydrogel or hydrogel matrix is from about 100 pm to about 2000 pm. In some embodiments, the thickness of the hydrogel or hydrogel matrix is from about 100 μm to about 1500 pm. In some embodiments, the thickness of the hydrogel or hydrogel matrix is from about 100 pm to about 1000 pm. In some embodiments, the thickness of the hydrogel or hydrogel matrix is from about 100 pm to about 950 pm. In some embodiments, the thickness of the hydrogel or hydrogel matrix is from about 100 μm to about 900 pm. In some embodiments, the thickness of the hydrogel or hydrogel matrix is from about 100 μm to about 850 μm. In some embodiments, the thickness of the hydrogel or hydrogel matrix is from about 100 pm to about 800 pm. In some embodiments, the thickness of the hydrogel or hydrogel matrix is from about 100 pm to about 750 pm. In some embodiments, the thickness of the hydrogel or hydrogel matrix is from about 100 pm to about 700 pm. In some embodiments, the thickness of the hydrogel or hydrogel matrix is from about 100 μm to about 650 μm. In some embodiments, the thickness of the hydrogel or hydrogel matrix is from about 100 μm to about 600 μm. In some embodiments, the thickness of the hydrogel or hydrogel matrix is from about 100 pm to about 550 μm. In some embodiments, the thickness of the hydrogel or hydrogel matrix is from about 100 μm to about 500 pm. In some embodiments, the thickness of the hydrogel or hydrogel matrix is from about 100 pm to about 450 pm. In some embodiments, the thickness of the hydrogel or hydrogel matrix is from about 100 pm to about 400 pm. In some embodiments, the thickness of the hydrogel or hydrogel matrix is from about 100 pm to about 350 pm. In some embodiments, the thickness of the hydrogel or hydrogel matrix is from about 100 μm to about 300 pm. In some embodiments, the thickness of the hydrogel or hydrogel matrix is from about 100 μm to about 250 μm. In some embodiments, the thickness of the hydrogel or hydrogel matrix is from about 100 μm to about 200 μm. In some embodiments, the thickness of the hydrogel or hydrogel matrix is from about 100 pm to about 150 pm. In some embodiments, the thickness of the hydrogel or hydrogel matrix is from about 300 pm to about 600 pm. In some embodiments, the thickness of the hydrogel or hydrogel matrix is from about 400 μm to about 500 μm.

In some embodiments, the hydrogel or hydrogel matrix comprises one or more synthetic polymers. In some embodiments, the hydrogel or hydrogel matrix comprises one or more of the following synthetic polymers: polyethylene glycol (polyethylene oxide), polyvinyl alcohol, poly-2-hydroxyethyl methacrylate, polyacrylamide, silicones, and any derivatives or combinations thereof.

In some embodiments, the hydrogel or hydrogel matrix comprises one or more synthetic and/or natural polysaccharides. In some embodiments, the hydrogel or hydrogel matrix comprises one or more of the following polysaccharides: hyaluronic acid, heparin sulfate, heparin, dextran, agarose, chitosan, alginate, and any derivatives or combinations thereof.

In some embodiments, the hydrogel or hydrogel matrix comprises one or more proteins and/or glycoproteins. In some embodiments, the hydrogel or hydrogel matrix comprises one or more of the following proteins: collagen, gelatin, elastin, titin, laminin, fibronectin, fibrin, keratin, silk fibroin, and any derivatives or combinations thereof.

In some embodiments, the hydrogel or hydrogel matrix comprises one or more synthetic and/or natural polypeptides. In some embodiments, the hydrogel or hydrogel matrix comprises one or more of the following polypeptides: polylysine, polyglutamate or polyglycine.

In some embodiments, the hydrogel comprises one or a combination of polymers sletec from those published in Khoshakhlagh et al., “Photoreactive interpenetrating network of hyaluronic acid and Puramatrix as a selectively tunable scaffold for neurite growth” Acta Biomaterialia, Jan. 21, 2015.

Any hydrogel suitable for cell growth can be formed by placing any one or combination of polymers disclosed herein at a concentration and under conditions and for a sufficient time period sufficient to create two distinct densities of crosslinked polymers: one cell-penetrable and one cell-impenetrable. The polymers may be synthetic polymers, polysaccharides, natural proteins or glycoproteins and/or polypeptides such as those selected from below.

### Synthetic Polymers

Such as polyethylene glycol (polyethylene oxide), polyvinyl alcohol, poly-2-hydroxyethyl methacrylate, polyacrylamide, silicones, their combinations, and their derivatives.

Polysaccharides (Whether Synthetic or Derived from Natural Sources)

Such as hyaluronic acid, heparan sulfate, heparin, dextran, agarose, chitosan, alginate, their combinations, and their derivatives.

### Natural Proteins or Glycoproteins

Such as collagen, gelatin, elastin, titin, laminin, fibronectin, fibrin, keratin, silk fibroin, their combinations, and their derivatives.

### Polypeptides (Whether Synthetic or Natural Sources)

Such as polylysine, and all of the RAD and EAK peptides already listed.

The term “three-dimensional” or “3D” as used herein means, for example, a thickness of culture of cells such that there are at least three layers of cells growing adjacent to one another. In some embodiments, the term three-dimensional means that, in context of the disclosed systems, the neurites and/or axons are from about 10 to about 1000 microns in thickness or height. In some embodiments, the term three-dimensional means that, in context of the disclosed systems, the neurites and/or axons are from about 10 to about 100 microns in thickness or height.

The term “isolated neurons” refers to neuronal cells that have been removed or disassociated from an organism or culture from which they originally grow. In some embodiments isolated neurons are neurons in suspension. In some embodiments, isolated neurons are a component of a larger mixture of cells including a tissue sample or a suspension with non-neuronal cells. In some embodiments, neuronal cells have become isolated when they are removed from the animal from which they are derived, such as in the case of a tissue explant. In some embodiments isolated neurons are those neurons in a DRG excised from an animal. In some embodiments, the isolated neurons comprise at least one or a plurality cells that are from one species or a combination of the species chosen from: sheep cells, goat cells, horse cells, cow cells, human cells, monkey cells, mouse cells, rat cells, rabbit cells, canine cells, feline cells, porcine cells, or other non-human mammals. In some embodiments, the isolated neurons are human cells. In some embodiments, the isolated neurons are stem cells that are pre-conditioned to have a differentiated phenotype similar to or substantially similar to a human neuronal cell. In some embodiments, the isolated neurons are human cells. In some embodiments, the isolated neurons are stem cells that are preconditioned to have a differentiated phenotype similar to or substantially similar to a nonhuman neuronal cell. In some embodiments, the stem cells are selected from: mesenchymal stem cells, induced pluripotent stem cells, embryonic stem cells, hematopoietic stem cells, epidermal stem cells, stem cells isolated from the umbilical cord of a mammal, or endodermal stein cells.

The term “neurodegenerative disease” is used throughout the specification to describe a disease which is caused by damage to the central nervous system ad or peripheral nervous system. Exemplary neurodegenerative diseases which may be examples of diseases that could be studied using the disclosed model, system or device include for example, Parkinson's disease, Huntington's disease, amyotrophic lateral sclerosis (Lou Gehrig's disease), Alzheimer's disease, lysosomal storage disease (“white matter disease” or glial/demyelination disease, as described, for example by Folkerth, J. Neuropath. Exp. Neuro., 58, 9, September, 1999), Tay Sachs disease (beta hexosamimidase deficiency), other genetic diseases, multiple sclerosis, brain injury or trauma caused by ischemia, accidents, environmental insult, etc., spinal cord damage, ataxia and alcoholism. In addition, the present invention may be used to test the efficacy, toxicity, or neurodegenerative effect of agents on neuronal cells in culture for the study of treatments for neurodegenerative diseases. The term neurodegenerative diseases also includes neurodevelopmental disorders including for example, autism and related neurological diseases such as schizophrenia, among numerous others.

A “score” is a numerical value that may be assigned or generated after normalization of the value based upon the presence, absence, or quantity of wave components in a in vitro synaptic circuit. In some embodiments, the score is normalized in respect to a control data value.

As used herein, the term “threshold” refers to a defined value by which a normalized score can be categorized. By comparing to a preset threshold, a waveform or waveform component, with corresponding qualitative and/or quantitative data corresponding to a normalized score, can be classified based upon whether it is above or below the preset threshold.

The term “neuronal cells” as used herein refers to, for example, cells that comprise at least one or a combination of dendrites, axons, and somata, or, alternatively, any cell or group of cells isolated from nervous system tissue. In some embodiments, neuronal cells are any cell that comprises or is capable of forming an axon. In some embodiments, the neuronal cell is a Schwann cell, glial cell, neuroglia, cortical neuron, embryonic cell isolated from or derived from neuronal tissue or that has differentiated into a cell with a neuronal phenotype or a phenotype which is substantially similar to a phenotype of a neuronal cell, induced pluripotent stem cells (iPS) that have differentiated into a neuronal phenotype, or mesenchymal stem cells that are derived from neuronal tissue or differentiated into a neuronal phenotype. In some embodiments, neuronal cells are neurons from dorsal root ganglia (DRG) tissue, retinal tissue, spinal cord tissue, or brain tissue from an adult, adolescent, child or fetal subject. In some embodiments, neuronal cells are any one or plurality of cells isolated from the neuronal tissue of a subject. In some embodiments, the neuronal cells are mammalian cells. In some embodiments, the cells are human cells and/or rat cells. In some embodiments, the cells are non-human mammalian cells or derived from cells that are isolated from non-human mammals. If isolated or disassociated from the original animal from which the cells are derived, the neuronal cells may comprise isolated neurons from more than one species. In some embodiments, the spheroid are free of a DRG tissue.

In some embodiments, neuronal cells are one or more of the following: central nervous system neurons, peripheral nervous system neurons, sympathetic neurons, parasympathetic neurons, enteric nervous system neurons, spinal motor neurons, motor neurons, sensory neurons, autonomic neurons, somatic neurons, dorsal root ganglia, cholinergic neurons, GABAergic neurons, glutamatergic neurons, dopaminergic neurons, scrotonergic neurons, interneurons, adrenergic neurons, and trigeminal ganglia. In some embodiments, glial cells are one or more of the following: astrocytes, oligodendrocytes, Schwann cells, microglia, ependymal cells, radial glia, satellite cells, enteric glial cells, and pituyicytes. In some embodiments, immune cells are one or more of the following: macrophages, T cells, B cells, leukocytes, lymphocytes, monocytes, mast cells, neutrophils, natural killer cells, and basophils. In some embodiments, stem cells are one or more of the following: hematopoietic stem cells, neural stem cells, embryonic stem cells, adipose derived stem cells, bone marrow derived stem cells, induced pluripotent stem cells, astrocyte derived induced pluripotent stem cells, fibroblast derived induced pluripotent stem cells, renal epithelial derived induced pluripotent stem cells, keratinocyte derived induced pluripotent stem cells, peripheral blood derived induced pluripotent stem cells, hepatocyte derived induced pluripotent stem cells, mesenchymal derived induced pluripotent stem cells, neural stem cell derived induced pluripotent stem cells, adipose stem cell derived induced pluripotent stem cells, preadipocyte derived induced pluripotent stem cells, chondrocyte derived induced pluripotent stem cells, and skeletal muscle derived induced pluripotent stem cells. In some embodiments, spheroids may also include other cell types such as keratinocytes or endothelial cells.

The terms “neuronal cell culture medium” or simply “culture medium” as used herein can be any nutritive substance suitable for supporting the growth, culture, cultivating, proliferating, propagating, or otherwise manipulating neuronal cells. In some embodiments, the medium comprises neurobasal medium supplemented with nerve growth factor (NGF). In some embodiments, the medium comprises fetal bovine serum (FBS). In some embodiments, the medium comprises L-glutamine. In some embodiments, the medium comprises ascorbic acid in a concentration ranging from about 0.001% weight by volume to about 0.01% weight by volume. In some embodiments, the medium comprises ascorbic acid in a concentration ranging from about 0.001% weight by volume to about 0.008% weight by volume. In some embodiments, the medium comprises ascorbic acid in a concentration ranging from about 0.001% weight by volume to about 0.006% weight by volume. In some embodiments, the medium comprises ascorbic acid in a concentration ranging from about 0.001% weight by volume to about 0.004% weight by volume. In some embodiments, the medium comprises ascorbic acid in a concentration ranging from about 0.002% weight by volume to about 0.01% weight by volume. In some embodiments, the medium comprises ascorbic acid in a concentration ranging from about 0.003% weight by volume to about 0.01% weight by volume. In some embodiments, the medium comprises ascorbic acid in a concentration ranging from about 0.004% weight by volume to about 0.01% weight by volume. In some embodiments, the medium comprises ascorbic acid in a concentration ranging from about 0.006% weight by volume to about 0.01% weight by volume. In some embodiments, the medium comprises ascorbic acid in a concentration ranging from about 0.008% weight by volume to about 0.01% weight by volume. In some embodiments, the medium comprises ascorbic acid in a concentration ranging from about 0.002% weight by volume to about 0.006% weight by volume. In some embodiments, the medium comprises ascorbic acid in a concentration ranging from about 0.003% weight by volume to about 0.005% weight by volume.

In some embodiments, the hydrogel, hydrogel matrix, and/or neuronal cell culture medium comprises any one or more of the following components: artemin, ascorbic acid, ATP, 13-endorphin, BDNF, bovine calf serum, bovine serum albumin, calcitonin gene-related peptide, capsaicin, carrageenan, CCL2, ciliary neurotrophic factor, CX3CL1, CXCL1, CXCL2, D-serine, fetal bovine serum, fluorocitrate. formalin, glial cell line-derived neurotrophic factor, glial fibrillary acid protein, glutamate, IL-1, IL-1 a, IL-113, IL-6, IL-10, IL-12, IL-17, IL-18, insulin, laminin, lipoxins, mac-l-saporin, methionine sulfoximine, minocycline, neuregulin-1, neuroprotectins, neurturin, NGF, nitric oxide, NT-3, NT-4, persephin, platelet lysate, PMX53, Poly-D-lysine (PLL), Poly-L-lysine (PLL), propentofylline, resolvins, S100 calcium-binding protein B, selenium, substance P, TNF-a, type I-V collagen, and zymosan.

As described herein, the term “optogenetics” refers to a biological technique which involves the use of light to control cells in living tissue, typically neurons, that have been genetically modified to express light-sensitive ion channels. It is a neuromodulation method employed in neuroscience that uses a combination of techniques from optics and genetics to control and monitor the activities of individual neurons in living tissue, even within freely-moving animals, and to precisely measure the effects of those manipulations in real-time. The key reagents used in optogenetics are light-sensitive proteins. Spatially-precise neuronal control is achieved using optogenetic actuators like channelrhodopsin, halorhodopsin, and archaerhodopsin, while temporally-precise recordings can be made with the help of optogenetic sensors for calcium (Aequorin, Cameleon, GCaMP), chloride (Clomeleon) or membrane voltage (Mermaid). In some embodiments, neural cells modified with optogenetic actuators and/or sensors are used in the culture systems described herein.

The term “plastic” refers to biocompatible polymers comprising hydrocarbons. In some embodiments, the plastic is selected from the group consisting of: Polystyrene (PS), Poly acrylo nitrile (PAN), Poly carbonate (PC), polyvinylpyrrolidonc, polybutadienc (PVP), Polyvinyl butyral (PVB), Poly vinyl chloride (PVC), Poly vinyl methyl ether (PVME), poly lactic-co-glycolic acid (PLGA), poly(1-lactic acid), polyester, polycaprolactone (PCL), poly ethylene oxide (PEO), polyaniline (PANI), polyflourenes, polypyrroles (PPY), poly ethylene dioxythiophene (PEDOT), and a mixture of two or any two or more of the foregoing polymers.

In some embodiments, the plastic is a mixture of three, four, five, six, seven, eight or more polymers.

The term “seeding” as used herein refers to, for example, transferring an amount of cells into a new culture vessel. The amount may be defined and may use volume or number of cells as the basis of the defined amount. The cells may be part of a suspension.

The terms “sequence identity” as used herein refers to, in the context of two or more nucleic acids or polypeptide sequences, the specified percentage of residues that are the same over a specified region. The term is synonymous with “sequence homology” or sequences being “homologous to” another sequence. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

The term “solid substrate” as used herein refers to any substance that is a solid support that is free of or substantially free of cellular toxins. In some embodiments, the solid substrate comprise one or a combination of silica, plastic, and metal. In some embodiments, the solid substrate comprises pores of a size and shape sufficient to allow diffusion or non-active transport of proteins, nutrients, and gas through the solid substrate in the presence of a cell culture medium. In some embodiments, the pore size is no more than about 10, 9, 8, 7, 6, 5, 4, 3, 2 or 1 micron in diameter. One of ordinary skill could determine how big of a pore size is necessary based upon the contents of the cell culture medium and exposure of cells growing on the solid substrate in a particular microenvironment. For instance, one of ordinary skill in the art can observe whether any cultured cells in the system or device are viable under conditions with a solid substrate comprises pores of various diameters. In some embodiments, the solid substrate comprises a base with a predetermined shape that defines the shape of the exterior and interior surface. In some embodiments, the base comprises one or a combination of silica, plastic, ceramic, or metal and wherein the base is in a shape of a cylinder or in a shape substantially similar to a cylinder, such that the first cell-impenetrable polymer and a first cell-penetrable polymer coat the interior surface of the base and define a cylindrical or substantially cylindrical interior chamber; and wherein the opening is positioned at one end of the cylinder. In some embodiments, the base comprises one or a plurality of pores of a size and shape sufficient to allow diffusion of protein, nutrients, and oxygen through the solid substrate in the presence of the cell culture medium. In some embodiments, the solid substrate comprises a plastic base with a pore size of no more than 1 micron in diameter and comprises at least one layer of hydrogel matrix; wherein the hydrogel matrix comprises at least a first cell-impenetrable polymer and at least a first cell-penetrable polymer; the base comprises a predetermined shape around which the first cell-impenetrable polymer and at least a first cell-penetrable polymer physically adhere or chemically bond; wherein the solid substrate comprises at least one compartment defined at least in part by the shape of an interior surface of the solid substrate and accessible from a point outside of the solid substrate by an opening, optionally positioned at one end of the solid substrate. In some embodiments, where the solid substrate comprises a hollow interior portion defined by at least one interior surface, the cells in suspension or tissue explants may be seeded by placement of cells at or proximate to the opening such that the cells may adhere to at least a portion the interior surface of the solid substrate for prior to growth. The at least one compartment or hollow interior of the solid substrate allows a containment of the cells in a particular three-dimensional shape defined by the shape of the interior surface solid substrate encourages directional growth of the cells away from the opening. In the case of neuronal cells, the degree of containment and shape of the at least one compartment are conducive to axon growth from soma positioned within the at least one compartment and at or proximate to the opening. In some embodiments, the solid substrate is cylindrical, tubular or substantially tubular or cylindrical such that the interior compartment is cylindrical or partially cylindrical in shape. In some embodiments, the solid substrate comprises one or a plurality of branched tubular interior compartments. In some embodiments, the bifurcating or multiply bifurcating shape of the hollow interior portion of the solids is configured for or allows axons to grow in multiple branched patterns. When and if electrodes are placed at to near the distal end of an axon and at or proximate to a neuronal cell soma, electrophysiological metrics, such as intracellular action potential can be measured within the device or system. In some embodiments, the electrodes are operably linked to a voltmeter, ammeter and/or a device capable of generating a current on a length of wire physically connecting the electrodes to the voltmeter, ammeter and/or device.

The disclosure relates to properly stuff hydrogel that comprises a mixture of both cell penetrable and cell impenetrable polymers. In some embodiments, the hydrogel comprises from about 10% to about 20% PEG and has a total modulus from about 0.1 to about 200 Pa. In some embodiments, the hydrogel has a modulus of about 0.5 Pa. In some embodiments, the hydrogel has a modulus of about 10 Pa. In some embodiments, the hydrogel has a modulus of about 50 Pa. In some embodiments, the hydrogel has a modulus of about 75 Pa. In some embodiments, the hydrogel has a modulus of about 90 Pa. In some embodiments, the hydrogel has a modulus of about 100 Pa. In some embodiments, the hydrogel has a modulus of about 125 Pa. In some embodiments, the hydrogel has a modulus of about 150 Pa. In some embodiments, the hydrogel has a modulus of about 175 Pa. In some embodiments, the hydrogel has a modulus of about 200 Pa. In some embodiments, the hydrogel has a modulus of no more than about 230 Pa.

### Spheroids

As used herein, a “spheroid” or “cell spheroid” can be, for example, any grouping of cells in a three-dimensional shape that generally corresponds to an oval or circle or convex or concave arc rotated about one of its principal axes, major or minor, and includes three-dimensional egg shapes, oblate and prolate spheroids, spheres, lens-shaped or substantially equivalent shapes.

A spheroid of the present invention can have any suitable width, length, thickness, and/or diameter. In some embodiments, a spheroid may have a width, length, thickness, and/or diameter in a range from about 150 microns to about 50,000 microns, or any range therein, such as, but not limited to, from about 100 microns to about 900 microns, about 100 microns to about 700 microns, about 300 microns to about 600 microns, about 400 microns to about 500 microns, about 500 microns to about 1,000 microns, about 600 microns to about 1,000 microns, about 700 microns to about 1,000 microns, about 800 microns to about 1,000 microns, about 900 microns to about 1,000 microns, about 750 microns to about 1,500 microns, about 1,000 microns to about 5,000 microns, about 1,000 microns to about 10,000 microns, about 2,000 to about 50,000 microns, about 25,000 microns to about 40,000 microns, or about 3,000 microns to about 15,000 microns. In some embodiments, a spheroid may have a width, length, thickness, and/or diameter of about 50 microns, 100 microns, 200 microns, 300 microns, 400 microns, 500 microns, 600 microns, 700 microns, 800 microns, 900 microns, 1,000 microns, 5,000 microns, 10,000 microns, 20,000 microns, 30,000 microns, 40,000 microns, or 50,000 microns. In some embodiments, a plurality of spheroids are generated, and each of the spheroids of the plurality may have a width, length, thickness, and/or diameter that varies by less than about 20%, such as, for example, less than about 15%, 10%, or 5%. In some embodiments, each of the spheroids of the plurality may have a different width, length, thickness, and/or diameter within any of the ranges set forth above.

The cells in a spheroid may have a particular orientation. In some embodiments, the spheroid may comprise an interior core and an exterior surface. In some embodiments, the spheroid may be hollow (i.e., may not comprise cells in the interior). In some embodiments, the interior core cells and the exterior surface cells are different types of cell. In some embodiments, the interior core comprises a magnetic nanoparticle.

The spheroids may vary in their stiffness, e.g., as measured by elastic modulus (Pascals; Pa). In certain embodiments, the elastic moduli of the spheroids are in a range from about 100 Pa to about 10,000 Pa, e.g., from about 100 Pa to about 12,000 Pa or from about 100 Pa to about, 4800 Pa. In some embodiments, the elastic moduli of the spheroids may be about 1200 Pa. As another example, the spheroid modulus may vary from about at least 10 Pa, at least about 100 Pa., at least about 150 Pa, at least about 200 Pa, or at least about 450 Pa. In some embodiments, the composition or device of the disclosure comprises one or a plurality of wells and each well comprises one or a plurality of different spheroids, a first, second, third, fourth or fifth or more population of spheroids. In one embodiment, the first spheroid comprises an elastic modulus from about 100 Pa to about 300 Pa, and the second spheroid comprises an elastic modulus from about 400 Pa to about 800 Pa. In another example, the first spheroid is characterized by an elastic modulus from about 50 to about 200 Pa, and a second spheroid is characterized by an elastic modulus from about 250 Pa to about 500 Pa.

In some embodiments, spheroids may be made up of one, two, three or more different cell types, including one or a plurality of neuronal cell types and/or one or a plurality of stem cell types. In some embodiments, the interior core cells may be made up of one, two, three, or more different cell types. In some embodiments, the exterior surface cells may be made up of one, two, three, or more different cell types.

In some embodiments, the spheroids comprise at least two types of cells. In some embodiments the spheroids comprise neuronal cells and non-neuronal cells. In some embodiments, the spheroids comprise neuronal cells and astrocytes at a ratio of about 5:1, 4:1, 3:1, 2:1 or 1:1 of neuronal cells to astrocytes. In some embodiments, the spheroids comprise neuronal cells and non-neuronal cells at a ratio of about 5:1, 4:1, 3:1, 2:1 or 1:1. In some embodiments, the spheroids comprise neuronal cells and non-neuronal cells at a ratio of about 1:5: 1:4, 1:3, or 1:2. Any combination of cell types disclosed herein may be used in the above-identified ratios within the spheroids of the disclosure.

Depending on the particular embodiment, groups of cells may be placed according to any suitable shape, geometry, and/or pattern. In some embodiments, the cells are arranged in a sphere across the surface area of a bead or nanoparticle with a solid or hollow core. For example, independent groups of cells may be deposited as spheroids, and the spheroids may be arranged within a three dimensional grid, or any other suitable three dimensional pattb em. The independent spheroids may all comprise approximately the same number of cells and be approximately the same size, or alternatively, different spheroids may have different numbers of cells and different sizes. In some embodiments, multiple spheroids may be arranged in shapes such as an L or T shape, radially from a single point or multiple points, sequential spheroids in a single line or parallel lines, tubes, cylinders, toroids, hierarchically branched vessel networks, high aspect ratio objects, thin closed shells, organoids, or other complex shapes which may correspond to geometries of tissues, vessels or other biological structures.

Any suitable physiological response of the spheroid may be determined, evaluated, measured, and/or identified in a method of the present disclosure. In some embodiments, 1, 2, 3, 4, or more physiological response(s) of the spheroid may be determined, evaluated, measured, and/or identified in a method of the present disclosure. In some embodiments, the physiological response of the spheroid may be a change in morphology for the spheroid. The method may comprise determining a change in morphology for the spheroid, which may include estimating at least one morphology parameter prior to contacting the spheroid with an agent, such as a chemical and/or biological compound, estimating the at least one morphology parameter after contacting the spheroid with the testing agent, and calculating the difference between the at least one morphology parameter prior to and after contacting the spheroid with the agent to provide the change in morphology for the spheroid. In some embodiments, the physiological response of the spheroid may be the spheroid shrinking or swelling in response to contact with a testing agent. Morphology of the spheroid may be determined using any methods known to those of skill in the art, such as, but not limited to, quantifying eccentricity and/or cross sectional area.

In some embodiments, the physiological response of the spheroid may be a change in volume for the spheroid. The method may comprise determining a change in volume for the spheroid, which may include estimating a first volume prior to contacting the spheroid with an agent, estimating a second volume after contacting the spheroid with the testing agent, and calculating the difference between the first volume and the second volume to provide the change in volume for the spheroid. In some embodiments, the physiological response of the spheroid may be the spheroid shrinking or swelling in response to contact with an testing agent.

The test agent may be any suitable compound, such as, for example, an organic compound, a small molecule compound (e.g., a small molecule organic compound), a protein, an antibody, an oligonucleotide (e.g., DNA and/or RNA), a gene therapy vehicle (e.g., a viral vector) and any combination thereof. One or more (e.g., 1, 2, 3, 4, 5, or more) agents may be used in a method of the present invention. For example, a method of the present invention may comprise contacting a spheroid of the present invention with two or more different agents. In some embodiments, a method of the present invention may modulate an activity in a spheroid indirectly, such as, for example, by contacting a spheroid of the present invention with a gene therapy vehicle (e.g., a viral vector). In some embodiments, the test agent is an opioid or small molecule with known or suspected analgesic properties.

The disclosure relates to a system comprising one or a plurality of spheroids. In some embodiments, the system comprise at least a first and a second spheroid, the first spheroid comprising one or plurality of cells from a dorsal root ganglia animal, and the second spheroid comprising one or a plurality of cells from a spinal cord of an animal.

The disclosure relates to a system comprising a first spheroid in fluid communication with a second spheroid. In some embodiments, the first and second spheroids are in physical contact with each other via a bundle of axons. In some embodiments, the bundle comprise 3, 4, 5, 6, 7, 8, 9, 10 or more axons. In some embodiments, the bundle comprises at least 10. 20, 30, 40, 50, 60, 70, 80, 90 or 100 axons.

The present disclosure relates to a composition comprising at least a first spheroid of cells comprising one or a combination of cells and/or tissues chosen from: a neuronal cell, nervous system ganglia, a stem cell, and an immune cell. In some embodiments, the spheroid comprises a tissue chosen from: a dorsal root ganglia and a trigeminal ganglia. In some embodiments, the spheroid comprises one or a plurality of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, astrocytes, oligodendrocytes, Schwann cells, microglia, ependymal cells, radial glia, satellite cells, enteric glial cells, and pituyicytes. In some embodiments, the spheroid comprises one or a plurality of glial cells. In some embodiments, the spheroid comprises one or a plurality of embryonic cells. In some embodiments, the spheroid comprises one or a plurality of mesenchymal stem cells. In some embodiments, the spheroid comprises one or a plurality of cells derived from an induced pluripotent stem cell. In some embodiments, the spheroid comprises one or a plurality of parasympathetic neurons. In some embodiments, the spheroid comprises one or a plurality of spinal motor neurons. In some embodiments, the spheroid comprises one or a plurality of central nervous system neurons. In some embodiments, the spheroid comprises one or a plurality of peripheral nervous system neurons. In some embodiments, the spheroid comprises one or a plurality of enteric nervous system neurons. In some embodiments, the spheroid comprises one or a plurality of motor neurons. In some embodiments, the spheroid comprises one or a plurality of sensory neurons. In some embodiments, the spheroid comprises one or a plurality of interneurons. In some embodiments, the spheroid comprises one or a plurality of cholinergic neurons. In some embodiments, the spheroid comprises one or a plurality of GAB Aergic neurons. In some embodiments, the spheroid comprises one or a plurality of glutamergic neurons, In some embodiments, the spheroid comprises one or a plurality of dopaminergic neurons. In some embodiments, the spheroid comprises one or a plurality of serotonergic neurons. In some embodiments, the spheroid comprises one or a plurality of trigeminal ganglion cells. In some embodiments, the spheroid comprises one or a plurality of astrocytes. In some embodiments, the spheroid comprises one or a plurality of oligodendrocytes. In some embodiments, the spheroid comprises one or a plurality of Schwann cells. In some embodiments, the spheroid comprises one or a plurality of microglial cells. In some embodiments, the spheroid comprises one or a plurality of ependymal cells. In some embodiments, the spheroid comprises one or a plurality of radial glia. In some embodiments, the spheroid comprises one or a plurality of satellite cells. In some embodiments, the spheroid comprises one or a plurality of enteric glial cells. In some embodiments, the spheroid comprises one or a plurality of pituyicytes.

In some embodiments, the spheroid comprises one or a plurality of one or combination of immune cells chosen from: a T cell, B cell, macrophage and astrocytes. In some embodiments, the spheroid comprises one or a plurality of one or a combination of stem cells chosen from: an embryonic stem cell, a mesenchymal stem cell, and an induced pluripotent stem cell. In some embodiments, the neuronal cell is derived from a stem cell chosen from: an embryonic stem cell, a mesenchymal stem cell, and an induced pluripotent stem cell.

Embodiments include each of the above-mentioned cell types with each other individually or in combination.

In some embodiments, the spheroid has a diameter from about 200 microns to about 700 microns. In some embodiments, the spheroid has a diameter from about 150 microns to about 800 microns. In some embodiments, the spheroid has a diameter of about 200 microns. In some embodiments, the spheroid has a diameter of about 300 microns. In some embodiments, the spheroid has a diameter of about 400 microns. In some embodiments, the spheroid has a diameter of about 500 microns. In some embodiments, the spheroid has a diameter of about 600 microns. In some embodiments, the spheroid has a diameter of about 700 microns. In some embodiments, the spheroid has a diameter of about 800 microns. In some embodiments, the spheroid has a diameter of about 900 microns. In some embodiments, the spheroid has a diameter of about 350 microns. In some embodiments, the spheroid has a diameter of about 450 microns. In some embodiments, the spheroid has a diameter of about 550 microns. In some embodiments, the spheroid has a diameter of about 650 microns. In some embodiments, the spheroid comprises one or a plurality of neuronal cells and one or a plurality of Schwann cells at a ratio of cell types equal to about 4 neuronal cells for every 1 Schwann cell. In some embodiments, the spheroid comprises one or a plurality of neuronal cells and one or a plurality of astrocytes at a ratio of about 4 neuronal cells for every 1 astrocyte. In some embodiments, the spheroid comprises one or a plurality of neuronal cells and one or a plurality of astrocytes at a ratio of about 1 neuronal cell for every 1 astrocyte. In some embodiments, the spheroid comprises one or a plurality of neuronal cells and one or a plurality of Schwann cells at a ratio of about 10 neuronal cells for every 1 Schwann cell. In some embodiments, the spheroid comprises one or a plurality of neuronal cells and one or a plurality of glial cells at a ratio equal to about four neuronal cells for every 1 glial cell. In some embodiments, a first spheroid is electrically and physically connected by way of a plurality of from about 10 to about 100 or more neurites in a parallel directional bundle to a second spheroid. In some embodiments the first spheroid comprises central nervous system neurons and the second spheroid comprises sensory neurons, such that, when excited by a stimulus, electricity is conducted from the spheroids comprising the central nervous system neurons to the spheroid comprising the sensory neurons. In this way, the system simulates a afferent nerve fiber between two spheroids. In some embodiments, a first spheroid is electrically and physically connected by way of a plurality of from about 30 to about 100 or more neurites in a parallel directional bundle to a second spheroid. In some embodiments, a first spheroid is electrically and physically connected by way of a plurality of from about 50 to about 100 or more neurites in a parallel directional bundle to a second spheroid. In some embodiments, a first spheroid is electrically and physically connected by way of a plurality of from about 75 to about 150 or more neurites in a parallel directional bundle to a second spheroid. In some embodiments, a first spheroid is electrically and physically connected by way of a plurality of from about 100 to about 1000 or more neurites in a parallel directional bundle to a second spheroid. In some embodiments, the bundle of axons comprises a length of about 1 mm, 1.1 mm, 1.2 mm, 1.3 mm, 1.4, 1.5, 1.6, 1.7, 1.8, 1.9, 2.0, 2.1, 2.2, 2.3, 2.4, 2.5, 2.6, 2.7 or about 2.8 millimeters.

In some embodiments, any one or plurality of cells described herein are differentiated from induced pluripotent stem cells. In some embodiments, the spheroid are free of induced pluripotent stem cells and/or immune cells. In some embodiments, the spheroid are free of undifferentiated stem cells.

In some embodiments, the spheroid comprises no less than about 10,000, 20,000, 25,000, 30,000, 35,000, 40,000, 45,000, 50,000, 55,000, 60,000, 65,000, 70,000, or 75,000 cells. In some embodiments, the spheroid comprises no less than 75,000 cells. In some embodiments, the spheroid comprises no less than 65,000 cells. In some embodiments, the spheroid comprises no less than 60,000 cells. In some embodiments, the spheroid comprises no less than 100,000 cells. In some embodiments, the spheroid comprises no less than 125,000 cells. In some embodiments, the spheroid comprises no less than 150,000 cells. In some embodiments, the spheroid comprises no less than 175,000 cells. In some embodiments, the spheroid comprises no less than 200,000 cells. In some embodiments, the spheroid comprises no less than 225,000 cells. In some embodiments, the spheroid comprises no less than 250,000 cells. In some embodiments, the spheroid comprises no less than 12,500 cells. In some embodiments, the spheroid comprises from about 12,500 cells to about 250,000 cells. In some embodiments, the spheroid comprises from about 12,500 cells to about 100,000 cells. In some embodiments, the spheroid comprises from about 12,500 cells to about 75,000 cells. In some embodiments, the spheroid comprises from about 12,500 cells to about 29,000 cells.

In some embodiments, the spheroid further comprises one or a plurality of magnetic particles. In some embodiments, the magnetic particles comprise one or more hollow interiors. In some embodiments, the magnetic particles comprises one or more layers of polymer onto which the cells form a spheroid.

The present disclosure also relates to a system comprising: (i) a cell culture vessel comprising a hydrogel; (ii) one or a plurality of spheroids comprising one or plurality of neuronal cells and/or isolated tissue explants; (iii) an amplifier comprising a generator for electrical current; (iv) a voltmeter and/or ammeter; and (v) at least a first stimulating electrode and at least a first recording electrode; wherein the amplifier, voltmeter and/or ammeter, and electrodes are electrically connected to the each other via a circuit in which electrical current is fed to the at least one stimulating electrode from the amplifier and electrical current is received at the recording electrode and fed to the voltmeter and/or ammeter; wherein the stimulating electrode is positioned at or proximate to one or a plurality of soma of the neuronal cells and/or isolated tissue explants and the recording electrode is positioned at a predetermined distance distal to the soma, such that an electrical field is established across the cell culture vessel. In some embodiments, the spheroid is any of the spheroids described herein.

In some embodiments, the culture vessel comprises 96, 192, 384 or more interior chambers. In some embodiments, the 96, 192, 384 or more interior chambers comprise one or plurality of isolated Schwann cells and/or one or plurality of oligodendrocytes sufficiently proximate to the one or plurality of isolated tissue explants and/or the one or plurality of neuronal cells such that the Schwann cells or the oligodendrocytes deposit myelin to axon growth from the tissue explants and/or neuronal cells.

In some embodiments, the system further comprises a solid substrate onto which the hydrogel matrix is crosslinked, said solid substrate comprising at least one plastic surface with pores from about 1 micron to about 5 microns in diameter. In some embodiments, the solid substrate comprises a contiguous exterior surface and an interior surface, such solid substrate comprising at least one portion in a cylindrical or substantially cylindrical shape and at least one hollow interior defined at its edge by at least one portion of the interior surface, said interior surface comprising one or a plurality of pores from about 0.1 microns to about 1.0 microns in diameter wherein the hollow interior of the solid substrate is accessible from a point exterior to the solid substrate through at least one opening; wherein the hollow interior portion comprises a first portion proximate to the opening and at least a second portion distal to the opening; wherein the one or plurality of neuronal cells and/or the one or plurality of tissue explants are positioned at or proximate to the first portion of the hollow interior and are in physical contact with the hydrogel matrix, and wherein the second portion of the at least one hollow interior is in fluid communication with the first portion such that axons are capable of growth from the one or plurality of neuronal cells and/or the one or plurality of tissue explants into the second interior portion of the hollow interior. In some embodiments, the system or composition is free of a sponge. In some embodiments, the hydrogel comprises at least a first cell-impenetrable polymer and a first cell-penetrable polymer. In some embodiments, the at least one cell-impenetrable polymer comprises no greater than about 15% PEG and the at least one cell-penetrable polymer comprises from about 0.05% to about 1.00% of one or a combination of self-assembling peptides chosen from: RAD 16-I, RAD 16-II, EAK 16-I, EAK 16-II, and dEAK 16. In some embodiments, the composition is free of polyethylene glycol (PEG). In some embodiments, the hydrogel comprises a first region and a second region, the first region is formed in the shape of a cylinder or rectangular prism oriented with its longitudinal axis passing through the top and bottom of the cell culture vessel and each of either the cylinder or rectangular prism comprising a space defined by an inner surface of the cylinder or rectangular prism, said space and accessible by one or more openings through the top of the cell culture vessel; wherein the second region comprises a space formed in the shape of its interior walls with an opening on its side adjacent to and in fluid communication with the first region. In some embodiments, the hydrogel comprises at least 1% polyethylene glycol (PEG). In some embodiments, the system further comprises a cell medium comprising nerve growth factor (NGF) at a concentration from about 5 to about 20 picograms per milliliter and/or ascorbic acid in a concentration ranging from about 0.001% weight by volume to about 0.01% weight by volume.

In some embodiments, the spheroids are in culture for no less than about 3, 30, 90, or 365 days. In some embodiments, at least one portion of the solid substrate is cylindrical or substantially cylindrical such that at least one portion of the interior surface of the solid substrate defines a cylindrical or substantially cylindrical hollow interior chamber in which the spheroids are positioned. In some embodiments, the hydrogel comprises a series of two or more cavities in fluid communication with each other by a series of channels, at least one cavity comprising a spheroid and at least a second cavity comprising a second spheroid, suspension of cells, or DRG; wherein the spheroid and the second spheroid, suspension of cells, or DRG is connected by a three-dimensional axon. In some embodiments, the cavities are wells with a U-shaped or rounded wells positioned in a horizontal or substantially horizontal plane of the solid substrate with each channel comprises one or a plurality of axons connecting the one or plurality of spheroids.

In some embodiments, the one or plurality of spheroids comprises one or a plurality of neuronal cells with axonal growth from about 100 microns to about 500 microns in width and from about 0.11 to about 10000 microns in length. In some embodiments, the three-dimensional axon is at least about 10 microns in height at its lowest point or is at least three cellular monolayers in height.

In some embodiments, the one or plurality of spheroids comprises one or a plurality of neuronal cells with axonal growth from about 1 to about 10 millimeters in length, wherein the axonal growth comprises from about 50 to about 150 neurites in a parallel, bundle. In some embodiments, the parallel bundle unidirectionally grows from the first spheroid to the second spheroid, or the second spheroid to the first spheroid. In some embodiments, the parallel bundle unidirectionally grows from a spheroid comprising cells of the central nervous system to a spheroid comprising cells of the sensory nervous system. The connection between the two spheroids in the presence of a first and second electrode can form a circuit. Such a circuit can be excited or stimulated by controlled voltage drops of manipulable amplitudes, periodicities, latencies over time, to monitor the cells responses in vitro. In some embodiments such as circuit is an in vitro synapse.

In some embodiments, the system comprises a first spheroid comprising: (i) one or a plurality of neuronal cells; and/or (ii) one or a plurality of Schwann cells or oligodendrocytes; and a second spheroid comprising: (i) one or a plurality of peripheral neurons; wherein each spheroid is positioned in the cavity. In some embodiments, the system comprises a first, second and third cavity each configured to hold a spheroid and at least 50 microliters of cell culture medium, wherein the cavities are aligned such that the first cavity is positioned proximal to the second cavity and distal to the third cavity. In some embodiments, the system comprises at least a fourth cavity into which cavities are positioned in a pattern such that each cavity defines a corner of a square. In some embodiments, the cavities are aligned into a line such that axons originating from the first spheroid in the first cavity extend to the second cavity, and axons from the spheroid in the second cavity extend to the axons in the third cavity.

In some embodiments, any of the compositions, systems, or methods as described in PCT/US2015/050061 may be used in embodiments of the present disclosure.

In some embodiments, the methods relate to a method of manufacturing a system. culture plate or device for culturing cells, the method comprising obtaining a stem cell, such as a induce pluripotent stem cell, exposing the cell to one or plurality of cellular growth factors, differentiating the stem cells into a neuronal cell, and seeding the cell into a solid substrate comprising a first and/or second cavity or well. In some embodiments, the first and/or second cavity is a U bottom well, a curved-bottom well or flat-bottom well. In some embodiments, the method comprise seeding about 10, 15, 20, 25, 30, 35, 40, 45, 50, 55, 60, 65, 70, 75, 80, 90, 100, 125, 150, 175, 200, 225 or 250 thousand cells. In some embodiments, the step of seeding the cells comprises seeding one or a plurality f cells in a series of cavities or wells separated within a solid substrate and each cavity or well comprising cell culture medium. In some embodiments, the step of seeding the cavities or wells comprises seeding the cells in a pattern positioned within the solid substrate such that each well comprises a spheroid of cells and each spheroid is grown in a suspension or hanging drop format. In some embodiments, the method of manufacturing a system, culture plate or device for culturing cells comprises allowing the cells to culture undisturbed for sufficient time for the cells to spontaneously form one or a plurality of spheroids.

The disclosure also relates to a method of testing the toxicity of an agent by exposing an agent to one or a plurality of spheroids on or within a cavity or well within a solid substrate. In some embodiments, the methods further comprises allowing the agent to be exposed to the one or plurality of spheroids for a time sufficient for the agent to become absorbed by one or a plurality of cells of the one or plurality of spheroids and then measuring the viability of the cells through a recording, observation of morphological changes or a combination of both.

The disclosure also relates to the method of forming a spheroid of cells derived from stem cells or cells from the nervous system of a subject. In some embodiments, the method of forming a spheroid comprises (i) differentiating cells from a stem cell to a cell or plurality of cell types that are one or a combination of a neuronal cell, astrocyte, Schwann cell, or any other cell disclosed herein, and then (ii) mixing the one or plurality of cells for a time period sufficient to form a spheroid. In some embodiments, the method does not comprise a step of differentiating any cells after a spheroid is formed. In some embodiments, the methods are free of exposing the spheroid or any cell to one or a plurality of DRGs.

### Methods

The disclosure relates to a method of screening one or a plurality stimuli for neuromodulatory effect on cells with the disclosed compositions or systems, wherein the compositions or systems comprise (i) a first spheroid of cells in electrical communication with a second spheroid of cells; (ii) a first electrode proximate to the first spheroid; and (iii) a second electrode proximate to the second spheroid, and the method comprises:
(a) seeding one or a plurality of spheroids in a system or composition disclosed herein;
(b) positioning an electrode by or proximate to the spheroids;
(c) establishing a control waveform between and across a first and second spheroid;
(d) exposing the spheroids to a stimulus;
(e) measuring or detecting an experimental waveform in the presence of the stimulus;
(f) normalizing the experimental waveform by subtracting the magnitude and direction of the control waveform;
(g) correlating the difference between the magnitude and direction of the experimental waveform and the control waveform to a neuromodulatory effect of the stimulus. In some embodiments, the stimulus is electrical potential applied across the first and second electrodes proximate to the first and second spheroids, respectively; and the experimental waveform is a detectable electrical field potential across the spheroids. In some embodiments, the stimulus is a light or wavelength of light applied to one spheroid and the experimental waveform is the detectable waveform is calcium influx across the membrane of cells in the first or second spheroid. In some embodiments, the methods include a method of detecting a recording in vitro system comprising; (i) a first spheroid of cells in electrical communication with a second spheroid of cells; (ii) a first electrode proximate to the first spheroid; and (iii) a second electrode proximate to the second spheroid, the method comprising:
(i) applying a voltage across the first and second electrodes at the first electrode for a sufficient time period and with a sufficient voltage to generate a detectable control waveform from the second electrode;
(ii) detecting the control waveform from the second electrode.

In some embodiments, the control waveform comprises a first and a second, fast negative-going wave followed by a third positive-going wave. In some embodiments, the method further comprises a step of detecting a waveform associated with the field potential of the synapse in the presence of the test agent. In some embodiments, the method further comprises a step of analyzing the waveform associated with a field potential in the presence of the test agent with the control waveform to determine neuromodulatory effect. In some embodiments, the step of analyzing comprises comparing the duration of the first, second, or third peaks of the waveform associated with a field potential in the presence of a test agent with the duration of the first, second, or third peaks of the control wave form; and, if the duration of the first second or third peaks of the waveform are increased in the presence of the test agent, the test agent is an analgesic. In some embodiments, the test agent is an opioid.

In some embodiments, the step of establishing a control wave form comprises stimulating a first electrode at or proximate to a first spheroid comprising human cells no more frequently than about once every ten seconds. In some embodiments, the step of establishing a control wave form comprises stimulating a first electrode at or proximate to a first spheroid comprising human cells without creating a depression response in the waveform measurements.

Methods of the disclosure relate to a method further comprising establishing a control waveform between and across a first and second spheroid only after a step of detecting spontaneous, bursts of waveforms between the first and second spheroids. In some embodiments mammalian cells are about 1, 2, 3, 4, 5, 6 or more months in culture prior to the step (c). In some embodiments, the cells are human cells from about 3 to about 6 or about 6 months in culture prior to performing step (c). In some embodiments, the methods further comprise a step of allowing the spheroids to grow in culture for a time period sufficient to allow the spheroid to exhibit spontaneous and/or periodic waveforms prior to step (c). In some embodiments, step (f) is performed by subtracting the waveform from the pre-synaptic position from the waveform in the post-synaptic position prior to and after exposing the system/spheroids to a test agent.

In some embodiments, any of the compositions, systems, or methods as described in PCT/US2015/050061 may be used in embodiments of the present disclosure.

In some embodiments, the methods relate to a method of manufacturing a system. culture plate or device for culturing cells, the method comprising obtaining a stem cell, such as a induce pluripotent stem cell, exposing the cell to one or plurality of cellular growth factors, differentiating the stem cells into a neuronal cell, and seeding the cell into a solid substrate comprising a first and/or second cavity or well. In some embodiments, the first and/or second cavity is a U bottom well, a curved-bottom well or flat-bottom well. In some embodiments, the method comprise seeding about 10, 15, 20, 25, 30, 35, 40, 45, 50, 55, 60, 65, 70, 75, 80, 90, 100, 125, 150, 175, 200, 225 or 250 thousand cells. In some embodiments, the step of seeding the cells comprises seeding one or a plurality f cells in a series of cavities or wells separated within a solid substrate and each cavity or well comprising cell culture medium. In some embodiments, the step of seeding the cavities or wells comprises seeding the cells in a pattern positioned within the solid substrate such that each well comprises a spheroid of cells and each spheroid is grown in a suspension or hanging drop format. In some embodiments, the method of manufacturing a system, culture plate or device for culturing cells comprises allowing the cells to culture undisturbed for sufficient time for the cells to spontaneously form one or a plurality of spheroids.

The disclosure also relates to a method of testing the toxicity of an agent by exposing an agent to one or a plurality of spheroids on or within a cavity or well within a solid substrate. In some embodiments, the methods further comprises allowing the agent to be exposed to the one or plurality of spheroids for a time sufficient for the agent to become absorbed by one or a plurality of cells of the one or plurality of spheroids and then measuring the viability of the cells through a recording, observation of morphological changes or a combination of both.

The present disclosure also relates to a method of measuring or quantifying a neuromodulatory effect of an agent comprising: (a) culturing one or a plurality of spheroids in any of the compositions disclosed herein in the presence and absence of the agent; (b) applying a voltage potential across the one or a plurality of spheroids in the presence and absence of the agent; (c) measuring one or a plurality of electrophysiological metrics from the one or plurality of spheroids in the presence and absence of the agent; and (d) correlating the difference in one or a plurality of electrophysiological metrics through the one or plurality of spheroids to the neuromodulatory effect of the agent, such that a change in electrophysiological metrics in the presence of the agent as compared to the electrophysiological metrics measured in the absence of the agent is indicative of a neuromodulatory effect, and no change of electrophysiological metrics in the presence of the agent as compared to the electrophysiological metrics measured in the absence of the agent is indicative of the agent not conferring a neuromodulatory effect.

The present disclosure also relates to a method of measuring or quantifying a neuromodulatory effect of an agent comprising: (a) culturing one or a plurality of spheroids in any of the compositions disclosed herein in the presence and absence of the agent; (b) measuring and/or observing one or more morphometric changes of the one or plurality of spheroids in the presence and absence of the agent; and (c) correlating the one or more morphometric changes with the neuromodulatory effect of the agent, such that a change in morphometrics in the presence of the agent as compared to the morphometrics measured and/or observed in the absence of the agent is indicative of a neuromodulatory effect, and no change of morphometrics in the presence of the agent as compared to the morphometrics measured and/or observed in the absence of the agent is indicative of the agent not conferring a neuromodulatory effect.

The above-described methods can be implemented in any of numerous ways. For example, the embodiments may be implemented using a computer program product (i.e. software), hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device. Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. The disclosure also relates to a as a computer readable storage medium comprising executable instructions to perform any Additionally, such software may be written using any of a number of suitable programming languages and/or programming or scripting tools, and also may be compiled as executable machine language code or intermediate code that is executed on a framework or virtual machine. In some embodiments, the language code is Igor and includes any of the steps identified individually or in sequence in the Examples.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements. Also, the disclosure relates to various embodiments in which one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The disclosure also relates to a computer program product encoded on a computer-readable storage medium comprising instructions for: (a) identifying or quantifying one or a plurality of wave components from a dataset; (b) calculating a first normalized score or scores corresponding to a magnitude of one or plurality of wave components as compared to a control score. In some embodiments, the computer program product further comprises instructions for: (c) identifying whether a normalized score varies from a control values based upon a magnitude of change calculated between the normalized score and the control values. In some embodiments, the computer program product further comprises instructions for a step of importing a control and experimental dataset from a plurality of waveforms from a dataset prior to step (a). In some embodiments, the wave components are wave latency, amplitude, periodicity, wave length or a combination thereof over a given time period. In some embodiments, such wave components are measured over 1, 2, 3, 4, 5, 6, 7, 8, 9, 10, 15, 20, 30 or 40 or more minutes. In some embodiments, such wave components are measured over 1, 2, 3, 4, 5, 6, 7, 8, 9, 10, or more minutes but over 50 or 100 millisecond increments. In some embodiments, such wave components are measured in milliseconds over several 100 millisecond increments.

In some embodiments, the first normalized and second normalized scores calculated by the computer program product of the disclosure correspond to one or a plurality of waveforms measured from a circuit disclosed herein (such a circuit being active between an electrical source to a first electrode, through cells or spheroids of the disclosure and into the second electrode electrically connected to a voltmeter or ammeter or other device that monitors and displays waveform components. In some embodiments, the computer program product of the disclosure further comprises a step of calculating a first threshold relative to a first control dataset and calculating a second threshold relative to a second control dataset. In some embodiments, the computer program product of the disclosure further comprises (d) comparing the first normalized score to a first control threshold and comparing a second normalized score to a second control threshold; and (e) classifying an agent as neuromodulatory based upon results of comparing of step (b) and/or (d) relative to the first and/or second threshold, wherein each of steps (d) and (e) are performed after step (b), and wherein the first threshold is calculated relative to a first control dataset and the second threshold is calculated relative to a second control.

In some embodiments, the analyzing step (b) of the computer program product of the disclosure comprises: (i) determining an absence, presence or quantity of wave components within one or a plurality of agents exposed to cells or systems disclosed herein, wherein step (i) is performed prior to step (d), and wherein the normalized score of step (d) is based upon step (i). In some embodiments, the analyzing step (c) of the computer program product of the disclosure comprises: (ii) determining an absence, presence or quantity of waveform components assigned to a test agent, wherein step (ii) is performed prior to step (d), and wherein the normalized score of step (d) is based upon steps (ii).

The disclosure further relates to a system comprising: (i) any of the disclosed computer program product; and (ii) a processor operable to execute programs; and/or a memory associated with the processor.

The disclosure additionally relates to a system for identifying a neuromodulatory effect of a test agent on a synapse comprising: a processor operable to execute programs; a memory associated with the processor; a database associated with said processor and said memory; a program stored in the memory and executable by the processor, the program being operable for: ((a) identifying or quantifying one or a plurality of wave components from a dataset; (b) calculating a first normalized score or scores corresponding to a magnitude of one or plurality of wave components as compared to a control score. A “score” is a numerical value that may be assigned or generated after normalization of the value based upon the presence, absence, or quantity of wave components in a in vitro synaptic circuit. In some embodiments, the score is normalized in respect to a control data value. As used herein, the term “threshold” refers to a defined value by which a normalized score can be categorized. By comparing to a preset threshold, a subject, with corresponding qualitative and/or quantitative data corresponding to a normalized score, can be classified based upon whether it is above or below the preset threshold.

In some embodiments, the program stored in the memory and executable by the processor of the system of the disclosure is further operable for: (d) comparing the first normalized score to a first threshold and comparing a second normalized score to a second threshold; and (e) classifying the subject as being neuromodulatory based upon results of comparing of step (b) and/or (d) relative to the first and/or second threshold, wherein each of steps (d) and (e) are performed after step (b), and wherein the first threshold is calculated relative to a first control dataset and the second threshold is calculated relative to a second control. In some embodiments, the analyzing step (b) operated by the program of the disclosed system comprises: (i) determining an absence, presence or quantity of a waveform component assigned to a test agent, wherein step (i) is performed prior to step (e), and wherein the normalized score of step (d) is based upon step (i).

The disclosure relates to a computer-implemented method of determining the neurotoxicity or neuromodulatory activity of a test agent comprising any of steps (a) through (e) disclosed herein, whereby, if there is a variation between the normalized score and the control threshold, the variation comprises a neuromodulatory effect. In some embodiments, the test agent has neurotoxic effects if the variation between the normalized score and the control threshold is a negative magnitude suggesting the electrical activity of the cells and spheroid in the system has diminished.

Functions, operations, components and/or features described herein with reference to one or more embodiments, may be combined with, or may be utilized in combination with, one or more other functions, operations, components and/or features described herein with reference to one or more other embodiments, or vice versa.

Although the disclosure has been described with reference to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure.

The following examples are meant to be non-limiting examples of how to make and use the embodiments disclosed in this application. Any publications, patents or patent applications disclosed in the examples or the body of the specification are incorporated by reference in their entireties.

### EXAMPLES

### Fabrication of DRG-SCDH Microphysiological Cocultures

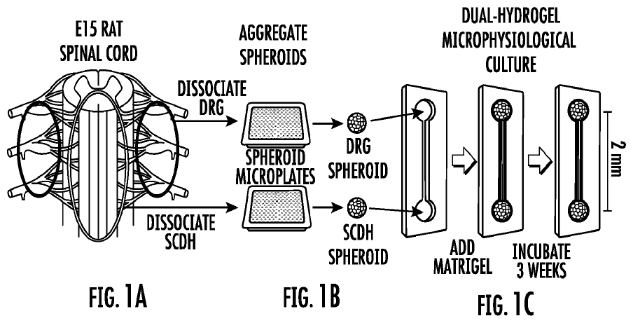

Our lab has previously developed embryonic rat microphysiological models of sensory nerve and validated their use for both in vitro neurotoxicity screening and in-depth comparative analysis of chemotherapy-induced peripheral neuropathy (36-40). Here, we advanced this model by coculturing two distinct neurospheroids, one from DRG and one from SCDH tissue, on either end of the culture system, optimized culture conditions to support 3-dimensional axonal growth both of neuronal types, and thoroughly characterized the resulting microphysiological interactions. DRG and SCDH tissues were harvested from an entire litter of gestation day 15 rat embryos. Tissue of each type was separately pooled, dissociated, and re-aggregated into a large homogenous batch of equivalent spheroids (FIG. 1). DRG and SCDH spheroids were seeded into opposite ends of a growth-restrictive polyethylene glycol hydrogel mold, which was then filled with a growth-permissive Matrigel-based hydrogel to provide a 3-dimensional extracellular matrix (ECM) for neurite migration. Over the course of three weeks, spheroids extended neurites and matured into a macroscopic, 3-dimensional, anisotropic, microphysiological tissue supporting long-distance nerve conduction and organized interaction between distinct nerve tissue types.

### Emergent Recapitulation of DRG-SCDH Unidirectional Circuit Structure and Tissue Morphology

We first set out to identify ECM conditions that promote emergence of the in vivo structure and function of DRG and SCDH tissue. Spheroids were cultured in several different gelatin methacrylate and Matrigel-based grown permissive hydrogel formulations of increasing stiffness (FIG. S1A-C). Spheroids remained comparably viable in all ECM formulations (FIG. S1D) indicating that any physiological differences among conditions are not a result of declining cell health. Consistent with previous reports (41-46), immunofluorescent staining of total nerve growth with the neuron-specific cytoskeletal protein, beta-III-tubulin, confirmed that stiff, gelatin methacrylate-based ECM promoted DRG neurite outgrowth and inhibited SCDH neurite outgrowth in monoculture (FIG. S1E). We further confirmed that DRG-SCDH coculture in stiff ECM produced the desired unidirectional neurite growth from DRG to SCDH (FIG. S1F) but surprisingly, bioelectric activity was undetectable in the SCDH spheroid region following electrical stimulation of DRG tissue which makes evaluation of synaptic physiology impossible. In contrast, soft Matrigel-based ECM permitted robust outgrowth of both DRG and SCDH neurites in monoculture (FIG. S1E) and robust detection of bioelectric SCDH field potentials in DRG-SCDH cocultures. Thus, stiff gelatin methacrylate ECM produced the desired unidirectional growth but not the desired physiological behavior while soft Matrigel ECM produced the desired physiological behavior but did not appear to produce the desired unidirectional growth pattern. These results illustrate how unexpected emergent microphysiological behaviors arise from interaction between both the cells in culture and the ECM composition and that these interactions are critical to accurate interpretation of data produced from MPS.

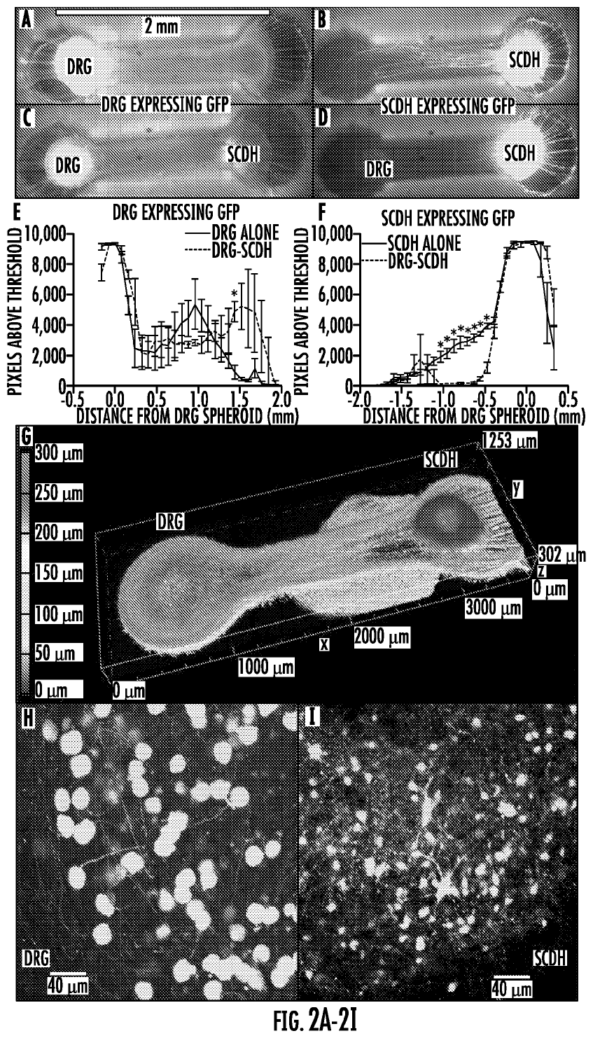

The apparent potential for bidirectional nerve growth in soft, Matrigel-based ECM could presumably confound the interpretation of field potential recordings in the SCDH spheroid because these potentials may be conflated with direct, electrically evoked activation of SCDH through distal neurite stimulation. Neurite tracing was performed to evaluate the extent of this potentially confounding bidirectional nerve growth. DRG or SCDH spheroids were infected with green fluorescent protein (GFP)-expressing adeno-associated viruses (AAVs) and cultured either alone or in the presence of an uninfected spheroid of the other type. Neurite outgrowth was then tracked over the three-week maturation period using green fluorescence to identify neurites extending from specified spheroids. Fluorescent neurite outgrowth was quantified after 17 days in microphysiological culture (FIG. 2A-I). Full statistical analysis of the neurite outgrowth assay, including all relevant F, p, and df values is presented in FIG. S1.

Compared to DRG monocultures, GFP-expressing DRG tissue in co-cultures showed little significant difference in neurite outgrowth except at a single location, approximately 1.5 mm distal from the DRG spheroid (p=0.0107), consistent with the front side of the unlabeled SCDH spheroid (FIG. 2E). In contrast, compared to SCDH monocultures, GFP-expressing SCDH tissue in co-cultures showed a consistently significant decrease in neurite growth between 0.5 and 1 mm distal from the SCDH spheroid, corresponding to the shared growth channel (FIG. 2F). These experiments demonstrated that although both DRG (FIG. 2A) and SCDH monocultures (FIG. 2B) extend significant nerve growth along the length of the growth permissive channel, SCDH nerve growth was significantly inhibited in DRG-SCDH coculture (FIG. 2D,F), while DRG nerve growth remained unaffected (FIG. 2C,E). The spontaneous recapitulation of unidirectional DRG-SCDH nerve growth in microphysiological culture is a novel, unexpected result and demonstrates an important emergent behavior of these tissues, the directed connectivity characteristic of in vivo nerve connections that underlay lower afferent pain signaling.

In subsequent cultures, AAV-GFP infection rate was reduced to about 20% to generate DRG-SCDH cocultures in which individual neuron cytoarchitecture could be resolved. 3-dimensional rendering of a z-stack through the mature culture demonstrated that DRG spheroids spontaneously adopted a flat tissue morphology (FIG. 2G, left) relative to SCDH spheroids that retained a more spheroidal morphology (FIG. 2G, right). Maximum intensity projections through higher magnification z-stacks of the DRG spheroid (FIG. 2H) and the SCDH spheroid (FIG. 2I) demonstrated that DRG soma were larger in size, with DRG soma averaging 18.4 μm and SCDH soma averaging 11.4 μm in diameter (Student's t-test, t=3.765, p=0.0197, df=4), and extended a single large axon into the ECM that is easily distinguished at 20× while SCDH neuronal soma were smaller in size and extended multiple, fine neurites forming a mesh-like neuritic arbor. Thus, these two tissues retain distinct characteristics of neuronal and tissue morphology in microphysiological culture that are consistent with in vivo DRG and SCDH nerve tissue.

### Asymmetrical Expression of Synaptic Proteins Suggests Unidirectional DRG-SCDH Connectivity

Qualitative immunofluorescent staining confirmed that both DRG and SCDH tissues expressed the neuronal markers, β-III-tubulin, and microtubule-associated protein 2 (MAP2) (FIG. S2C,I). The peripheral nerve specific marker, peripherin (FIG. S2D) was highly expressed in the DRG, but not SCDH, confirming peripheral and central nerve identity, respectively. The synaptic proteins, synapsin-I, postsynaptic density protein 95 (PSD95), and vesicular GABA transporter (vGAT), were highly expressed in the SCDH (FIG. S2F,G,J) suggesting the potential formation of functional anterograde DRG-SCDH synapses. However, relatively minimal expression of these synaptic proteins in the DRG spheroid region suggested limited potential for significant retrograde SCDH-DRG synapse formation.

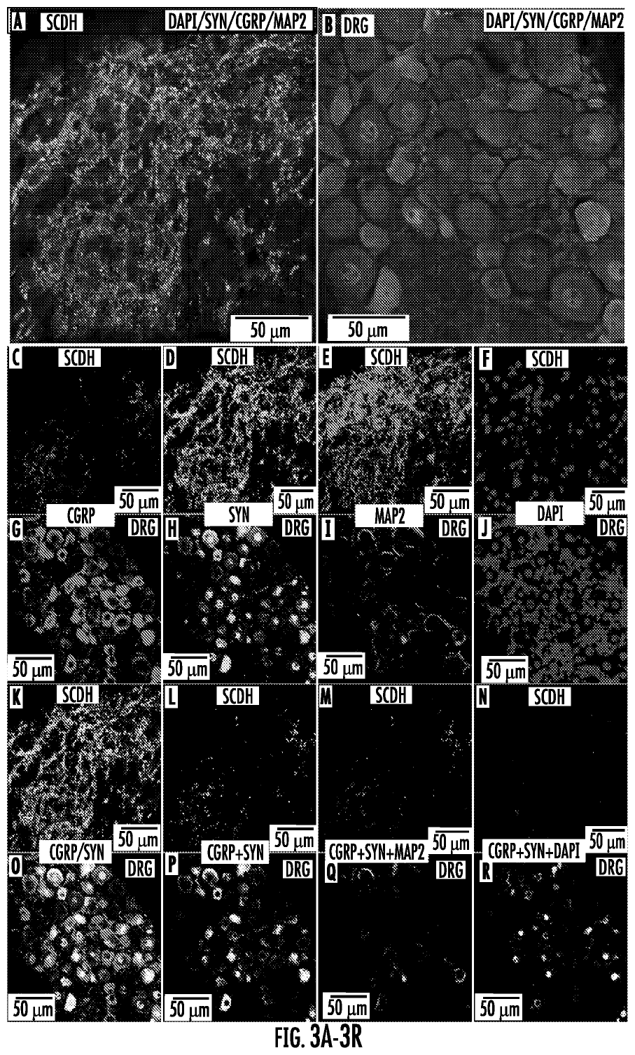

Analysis of colocalization of the presynaptic marker, synapsin-I; PNS-specific presynaptic marker, calcitonin gene-related peptide (CGRP); and the dendritic marker, MAP2, was performed to visualize the potential for synaptic connections in DRG-SCDH cocultures. When immunofluorescently stained for these three markers, a highly punctate staining pattern of synapsin-I, consistent with synaptic nerve terminals within the MAP2-stained dendritic arbor of SCDH, was observed upon high magnification imaging of immunofluorescently stained, cryosectioned microphysiological cocultures (FIG. 3A). Although all neurons in the DRG spheroid region were synapsin-I positive and a subset of neurons were CGRP positive, there was an absence of punctate staining in the DRG spheroid region suggesting an absence of synaptic connectivity (FIG. 3B). Colocalization analysis was performed on binarized images (FIG. 3C-J). Although CGRP staining density was higher in the DRG spheroid (FIG. 3G) than the SCDH spheroid (FIG. 3C) in the presented images, 42% of CGRP expression colocalized with synapsin-1 expression in SCDH (FIG. 3K,L), while only 24% of CGRP expression colocalized with synapsin-1 in the DRG spheroid (FIG. 3O,P). Furthermore, 39% of CGRP/synapsin-1 colocalized puncta in the SCDH spheroid also tri-colocalized with MAP2 expression (FIG. 3M), indicative of a synaptic connection, while only 3% of CGRP/synapsin-1 colocalized puncta also tri-colocalized with DAPI, indicative of intracellular expression of these synaptic proteins. In contrast, only 19% of CGRP/synapsin-1 colocalized pixels also tri-colocalized with MAP2 in the DRG spheroid, while a larger proportion, 35%, tri-colocalized with DAPI. Together these data suggest that DRG neurons are producing synaptic proteins and that these proteins are preferentially localized at putative synapses in the SCDH spheroid but preferentially localized at intracellular, non-synaptic sites in the DRG soma. The colocalization of punctate synaptic markers in the SCDH but not DRG spheroid region indicates the potential for appropriate anterograde DRG-SCDH but not retrograde SCDH-DRG synaptic connections.

### Optogenetics Confirm Emergence of Functional Unidirectional DRG-SCDH Synaptic Neurotransmission

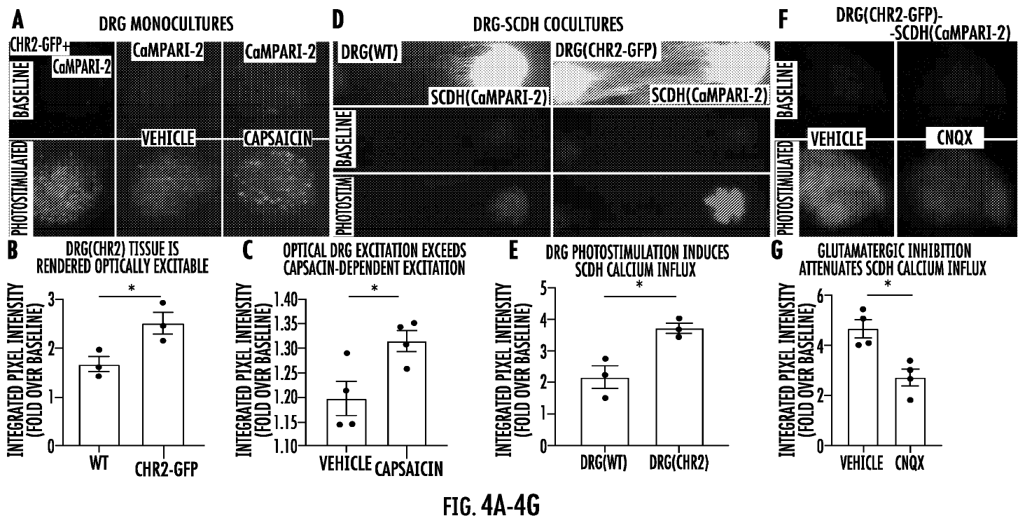

Viral expression of a channelrhodopsin-2-GFP (CHR2-GFP) fusion protein (47) renders nerves optically excitable while viral expression of the calcium integrator, CaMPARI-2 (48), renders neuronal calcium currents optically recordable. We first confirmed that viral expression of CHR2-GFP rendered microphysiological DRG tissue optically excitable. After co-expressing both CHR2-GFP and CaMPARI-2 in DRG monocultures, photostimulation-induced calcium influx recorded by CaMPARI-2 was significantly greater in DRG cultures expressing CHR2-GFP than baseline currents in cultures lacking CHR2-GFP (FIG. 4A,B [t-test: t=3.048, p=0.0381, df=4]). For comparison, treatment with capsaicin, which is often used as a physiologically-relevant stimulus for in vitro DRG neurons (49, 50), also induced significant calcium influx in DRG(CHR2-GFP+CaMPARI-2) monocultures (FIG. 4A,C [t-test: t=2.0851, p=0.0291, df=6]). Synchronized CHR2-induced calcium influx was larger than asynchronous capsaicin-induced calcium influx confirming that optical excitation of DRG tissue is well above the threshold for physiological significance (FIG. 4A-C).

We then confirmed that concerted optogenetic stimulation of DRG nerve tissue produces a robust physiological response in cocultured SCDH tissue. Six CaMPARI-2 expressing SCDH spheroids were cocultured with either optically excitable DRG tissue (n=3, denoted DRG(CHR2-GFP)-SCDH(CaMPARI-2) constructs) or wild-type DRG tissue (n=3, denoted DRG(WT)-SCDH(CaMPARI-2) constructs). DRG(WT) tissue did not respond to optical excitation and only baseline levels of calcium influx were recorded in the SCDH spheroid (FIG. 4D). In contrast, optical excitation of DRG tissue in DRG(CHR2-GFP)-SCDH(CaMPARI-2) constructs significantly increased calcium influx in SCDH tissue (FIG. 4D,E [t-test: t=3.881, p=0.0178, df=4]). This confirms that optical excitation in DRG tissue induces active calcium influx in cocultured SCDH tissue, consistent with synaptic transmission.

We finally confirmed that optically excitable DRG tissue activates calcium currents in SCDH tissue through glutamatergic synaptic transmission. Ten additional DRG(CHR2-GFP)-SCDH(CaMPARI-2) cultures were generated. Half of these cocultures were pretreated with the AMPA/Kainate receptor antagonist, CNQX, to inhibit glutamatergic neurotransmission while the other half were treated with vehicle alone (0.2% DMSO) for 15 minutes prior to photostimulation. CNQX pretreatment significantly reduced calcium influx in SCDH following optical excitation of DRG tissue relative to treatment with vehicle alone (FIG. 4F,G [t-test, t=3.945, p=0.0076, df=6]). This result confirms that photostimulated DRG nerve tissue excites SCDH nerve tissue through glutamatergic neurotransmission in this microphysiological coculture.

### Distinct and Replicable Local Field Potentials Recorded in Cocultures Reflect the Local Underlying Physiology

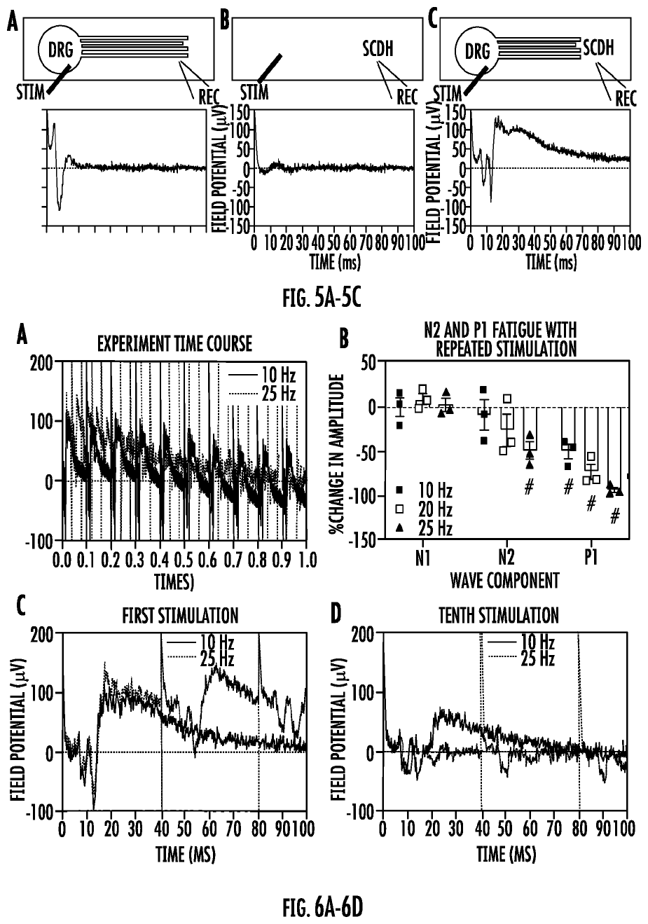

Extracellular field potential production was qualitatively evaluated throughout DRG and SCDH monocultures, DRG-DRG cocultures, SCDH-SCDH cocultures, and DRG-SCDH cocultures to identify a potential synaptic waveform specific to the DRG-SCDH configuration. Electrically-evoked compound action potentials (CAPs) were readily recorded in DRG spheroid monocultures. CAP conduction was detected both in the DRG spheroid following stimulation of the nerve region (FIG. S3A) and in the nerve region following stimulation of the DRG spheroid in mature DRG monocultures (FIG. 5A). Field potentials recorded in the spheroid region consisted of a large negative-going population spike followed by several smaller negative-going peaks while field potentials recorded in the nerve outgrowth consisted of a single similar large negative-going population spike lacking subsequent smaller peaks. Consistent with electrically evoked action potentials, these CAPs are insensitive to EDTA and CNQX but can be completely blocked by tetrodotoxin (FIG. S4).

Electrically-evoked CAP conduction was not detectable in the nerve (FIG. S3D) or spheroid region (FIG. 5B) of SCDH spheroid monocultures after direct stimulation, despite the observation that nerve growth extends the entire length of the growth-permissive channel (FIG. 2B). It is likely that the SCDH nerve tissue was activated by the electrical stimulation, but the resulting field potentials were not large or synchronous enough to be detected through extracellular recording.

Waveforms recorded in the DRG spheroid region of DRG-SCDH cocultures (FIG. S3H, recording A) were nearly identical to waveforms recorded in DRG monocultures (FIG. S3A) indicating that the DRG field potential was relatively unaffected by coculture with SCDH tissue. However, a consistent and unique waveform indicative of synaptic transmission emerged in SCDH spheroids after coculture with DRG tissue (FIG. 5C). This waveform was recorded in the SCDH after stimulation of DRG spheroids, was replicable, and was distinct from waveforms observed in DRG or SCDH monocultures, DRG-DRG cocultures, or SCDH-SCDH cocultures (FIG. S3A-F). This waveform consisted of two, fast negative-going population spikes (denoted N1 and N2) followed by a slower but prolonged positive-going wave (denoted P1). Stimulation of the distal DRG neurite growth in FIG. S3I confirms that this synaptic waveform was not conflated with electrically-evoked SDCH spheroid activation resulting from direct stimulation of distal SCDH neurite growth. This distal stimulation site was well beyond the range of SCDH neurite outgrowth observed in DRG-SCDH coculture (FIG. 2D) but still produced a SCDH spheroid waveform containing the same three components as more proximal stimulation sites, albeit with the longer latency and reduced amplitude expected from a more distal stimulation. The 2 mm stimulation site was found to be optimal as it produced the most synchronous waveform that permitted best resolution of the individual components and was therefore used for all subsequent experiments (FIG. S3I).

Fatigue Associated with Repeated Stimulation Further Distinguishes Synaptic Waveform Components

The synaptic nature of the unique DRG-SCDH waveform was first challenged with high frequency stimulation, which is expected to fatigue synaptically-based components more readily than electrically evoked components (51, 52). The DRG spheroid region of DRG-SCDH coculture constructs was sequentially stimulated for one second at increasing frequencies of 10 Hz, 20 Hz, and 25 Hz (FIG. 6A). The amplitude of N1, N2, and P1 was measured for each stimulation in the train, and amplitudes were normalized to initial amplitude to obtain a percent change in amplitude across the stimulation train (FIG. 6B). N1 amplitude remained unchanged across the first ten stimulations at all frequencies (FIG. 6B-D). In contrast, a significant reduction in N2 was observed at 25 Hz (one sample t-test vs 0%, t=5.031, p=0.0³73, df=2) and a significant reduction in P1 amplitude was observed at frequencies of 10 Hz and above (10 Hz: t=5.973 p=0.0373, df=2, 20 Hz: t=8.233 p=0.0144, df=2, 25 Hz: t=33.14, p=0.0009, df=2). These results suggest that N1 is a result of electrically-evoked CAP conduction while N2 and P1 result from synaptic transmission.

Selective Pharmacological Inhibition Dissociates Synaptic Waveform Components from Fiber Volley

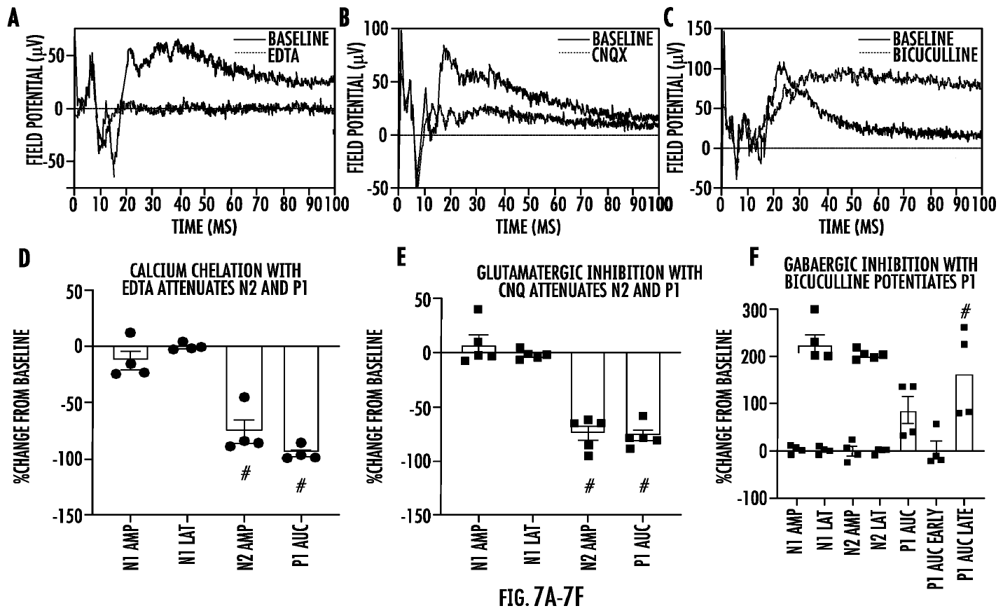

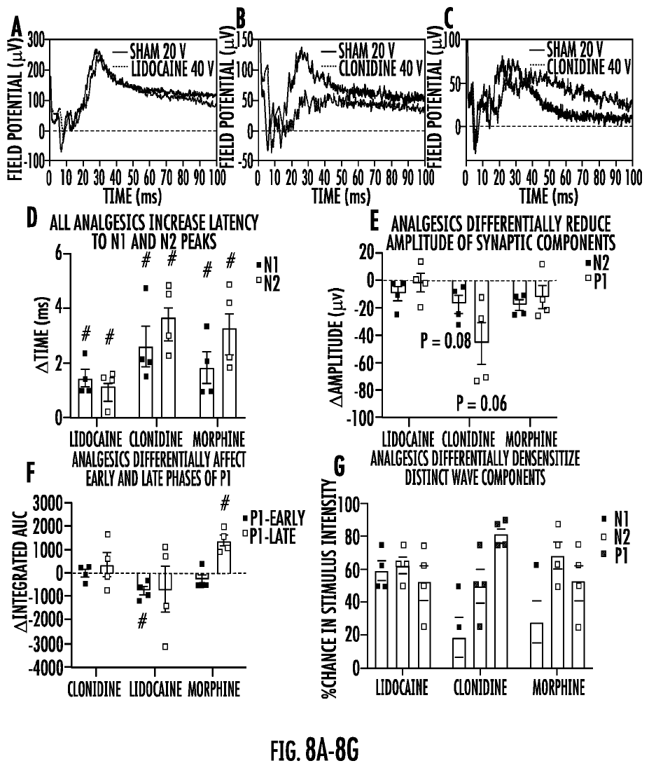

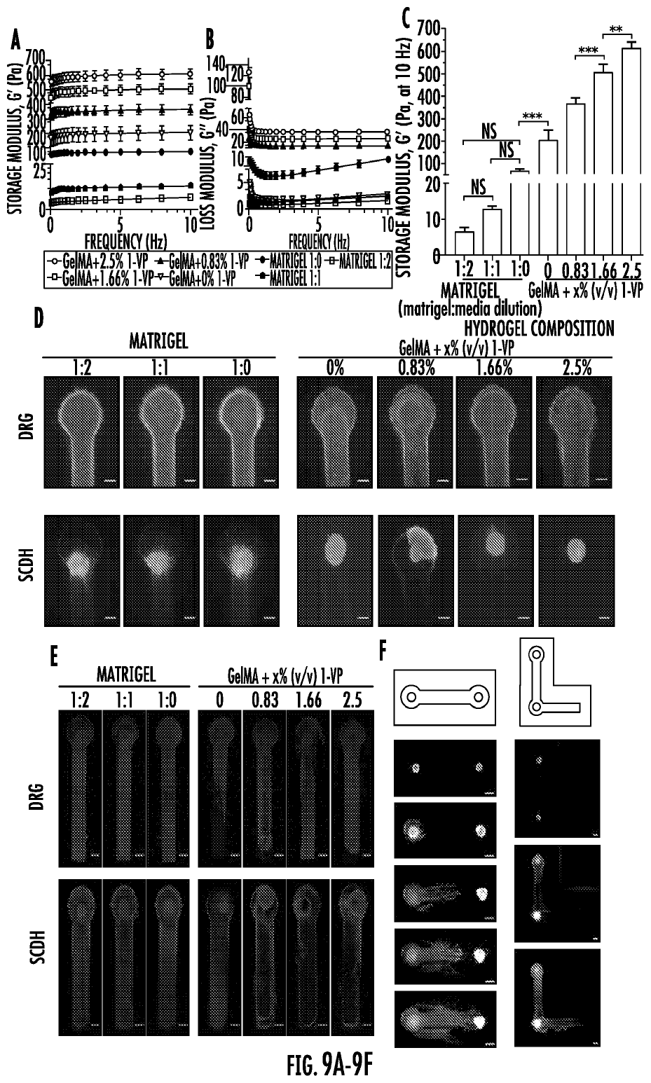

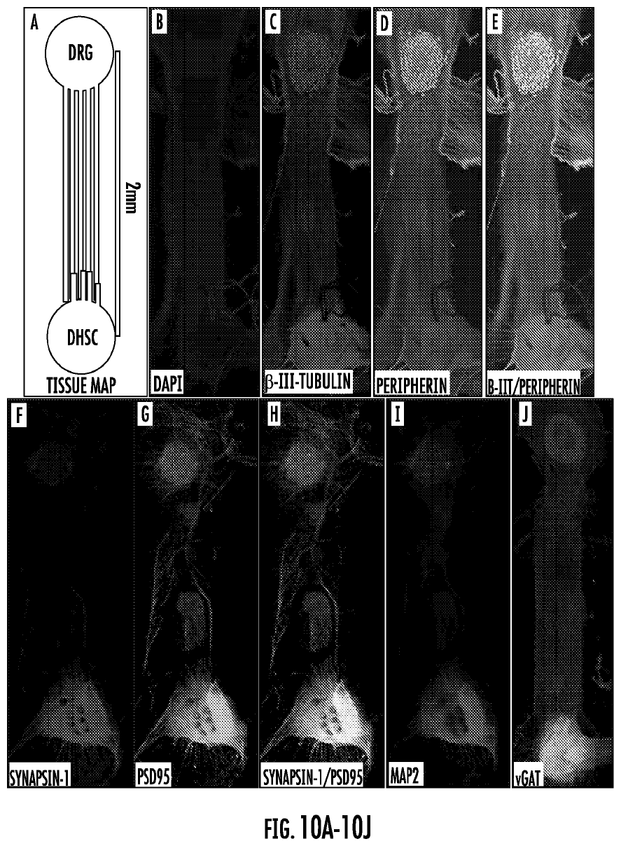

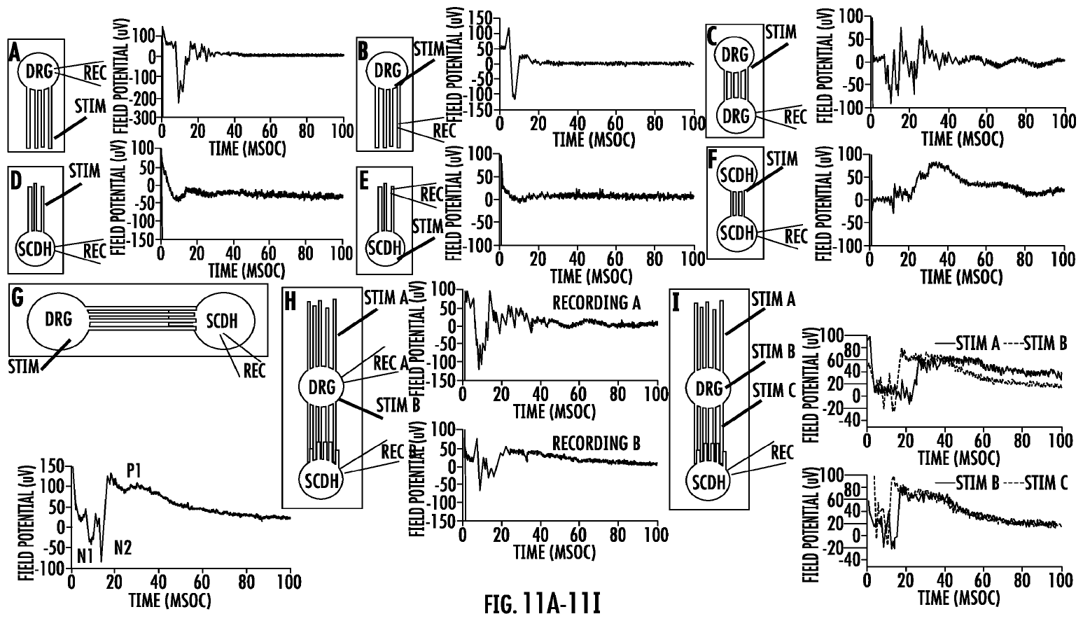

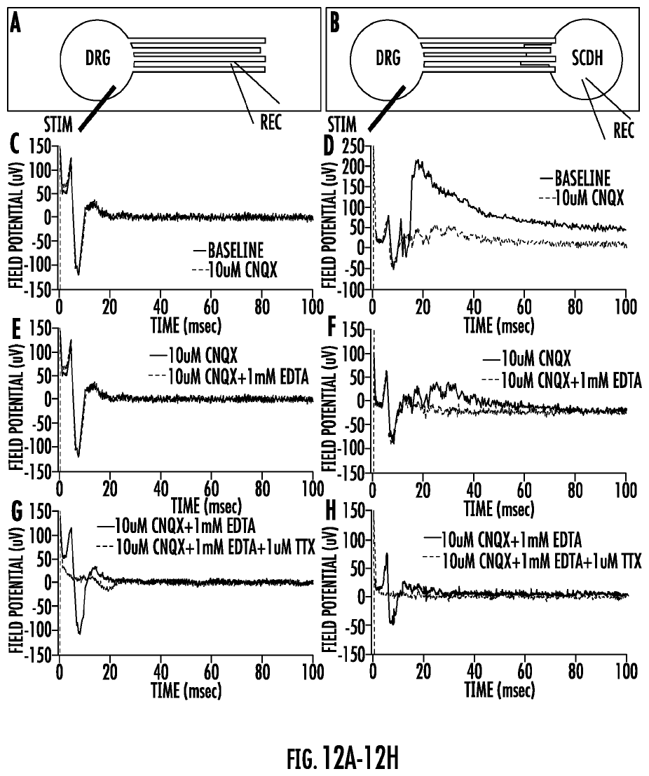

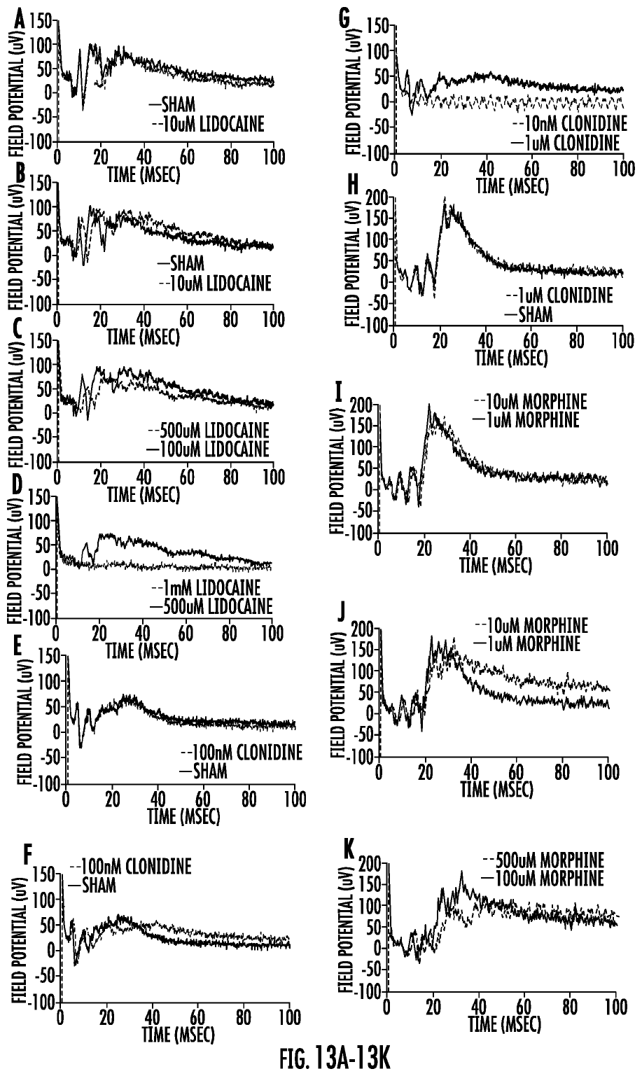

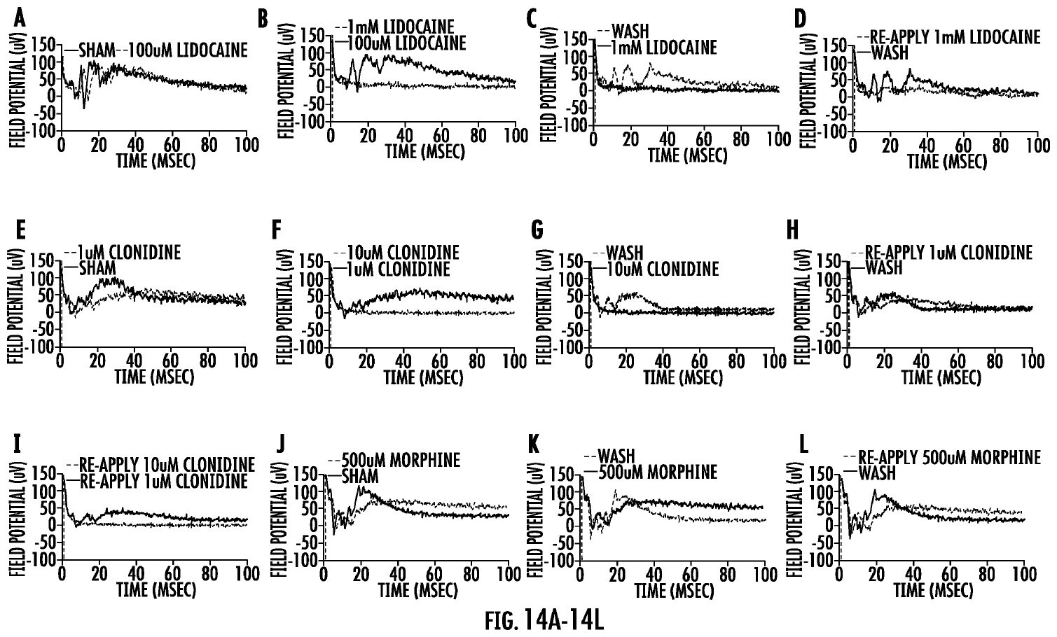

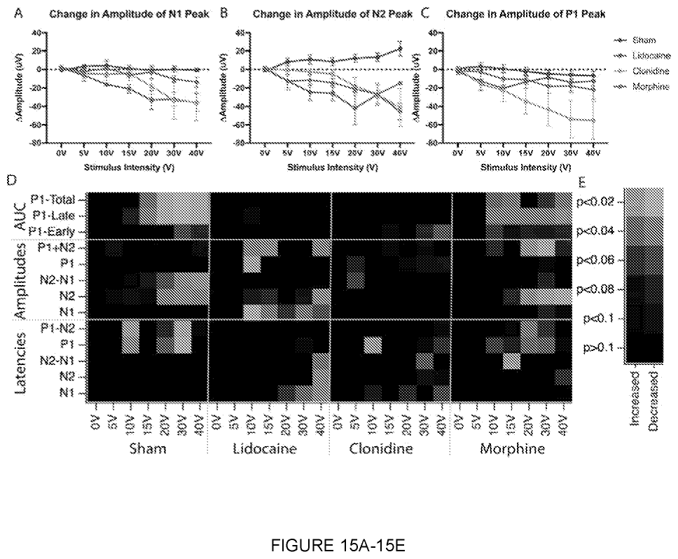

The synaptic basis of the unique DRG-SCDH waveform was further challenged through selective pharmacological inhibition of synaptic transmission. Bath application of EDTA chelates free calcium, preventing calcium-dependent release of synaptic vesicles. The AMPA/Kainate receptor antagonist, CNQX, was applied to specifically block glutamatergic neurotransmission, while the GABAA-receptor antagonist, bicuculline, was applied to specifically block GABAergic neurotransmission. Blockade of synaptic transmission with EDTA (FIG. 7A,D) completely blocked the N2 population spike (one sample t-test vs 0%, t=7.309, p=0.0053, df=3) as well as the P1 wave (t=30.53, p<0.0001, df=3). Similarly, blockade of glutamatergic neurotransmission with CNQX (FIG. 7B,E) significantly reduced N2 (t=11.23, p=0.0004, df=3) and P1 (t=15.42, p=0.0001, df=4). In contrast, blockade of GABAergic neurotransmission with bicuculline (FIG. 7C,F) specifically and significantly increased the late phase of the P1 wave (t=3.433, p=0.0415) while having no effect on N1 or N2 (FIG. 7C,F). None of these treatments affected the latency or amplitude of N1.

N1 was insensitive to high frequency stimulation (FIG. 6) and all modulators of synaptic activity (FIG. 7), but was completely blocked by the voltage-gated sodium channel inhibitor, tetrodotoxin (FIG. S4). This is consistent with direct recording of the electrically-evoked, concerted depolarization of presynaptic afferent DRG nerve fibers growing through the SCDH spheroid region, or fiber volley. In contrast, N2 and P1 fatigue under repeated stimulation and are not observed when synaptic vesicle release is prevented by calcium chelation, confirming they are synaptic in nature. N2 is negative-going, similar in size and time-locked to N1, inhibited by glutamatergic inhibition, but insensitive to GABAergic inhibition. This is consistent with a concerted primary field excitatory post-synaptic potential (fEPSP) resulting from glutamatergic neurotransmission from DRG to SCDH tissue. P1 is synaptically-evoked, but is delayed and prolonged relative to N1 and N2, continuing tens of milliseconds after termination of N2. This likely represents a recording of higher-order, multi-synaptic impulses propagating through the SCDH, which is prolonged by disinhibition after blockade of inhibitory neurotransmission with bicuculline. Together, these results strongly suggest that functional and organized synaptic physiology is an emergent behavior of DRG and SCDH tissue in microphysiological coculture.

### Common Analgesics Induce Overlapping but Distinct Effects on Synaptic Physiology

Preliminary qualitative dose-response experiments were performed to identify the lowest effective dose of each analgesic and to demonstrate the reversibility of drug effects with at least a partial washout (FIG. S5,6). The lowest effective dose was then replicated in four separate microphysiological cultures. A full stimulus-response curve was performed for each sample at baseline, after a sham treatment, and after analgesic treatment and the change in latency and amplitude of N1, N2, and P1 wave components as well as the integrated area under the curve (AUC) of P1 were calculated for each stimulus intensity at each stage of treatment.

Quantitative analysis of the stimulus-response curves revealed evidence of analgesic-induced attenuation of the fiber volley peak across stimulus intensities, suggesting that interpretation of the postsynaptic effect of analgesics is partially conflated with presynaptic desensitization (FIG. S7). Therefore, the maximum response observed after analgesic treatment was paired with the sham stimulation intensity that had previously produced an N1 fiber-volley peak of most similar amplitude and differences in the synaptic components of the waveform were compared between N1-matched traces to better isolate changes to the postsynaptic response to an input of similar intensity (FIG. 8A-C). Significant changes in waveform component metrics between N1-matched sham and analgesic traces were identified with a one sample t-test vs 0 (indicating no change) comprised of n=4 paired traces for each drug treatment (df=3) and all exact p-values resulting from this analysis are shown in Table S2. Statistical analysis confirmed that all three analgesics significantly increased the latency to N1 and N2 (FIG. 8D). Lidocaine had no additional significant effects on the shape of the synaptic waveform. In contrast, additional changes were evident after treatment with both clonidine and morphine. Clonidine preferentially decreased P1 amplitude while morphine significantly reduced N2 amplitude (FIG. 8E) and clonidine uniquely decreased the early phase of P1 while morphine uniquely increased the late phase of P1 (FIG. 8F). These results indicated that lidocaine acts primarily on presynaptic DRG tissue while clonidine and morphine affect the postsynaptic response in different ways independent of changes in presynaptic input strength.

Finally, the maximal post-treatment amplitude of each waveform component was independently matched with the associated pretreatment trace that evoked a response of most similar magnitude. The percent change in stimulus intensity required to evoke this response of similar amplitude was then calculated to independently quantify changes in the sensitivity of each distinct waveform component. Lidocaine uniquely decreased the sensitivity of the presynaptic fiber-volley, requiring approximately 60% greater stimulus intensity to observe an N1 peak of similar magnitude (FIG. 8G). A similar desensitization of N2 and P1 were observed indicating that the desensitization of N1 drives the desensitization of the dependent N2 and P1 peaks. In contrast, N2 and P1 were primarily desensitized after treatment with clonidine and morphine while no significant effect on N1 sensitivity was observed. These results confirm that lidocaine primarily desensitizes presynaptic DRG nerve tissue while clonidine and morphine directly desensitize the postsynaptic response.

### Discussion

Here, we present several converging lines of evidence indicating that emergent properties of microphysiological DRG-SCDH spheroid cocultures self-assemble an efficient and informative model of lower afferent pain signaling in our defined MPS. Microphysiological cocultures spontaneously assemble into a unidirectional circuit with extensive anterograde (DRG-to-SCDH) but limited retrograde (SCDH-to-DRG) neurite extension (FIG. 2A-F). DRG and SCDH spheroid tissue retain distinct morphologies that are consistent with in vivo DRG and SCDH tissue structure and cytoarchitecture (FIG. 2G-I). Colocalization of pre- and post-synaptic markers in the SCDH but not DRG spheroid region confirm the potential for anterograde but not retrograde synapse formation (FIG. 3). A specific and unique synaptic field potential composed of at least two, fast negative-going peaks (N1 and N2) followed by a prolonged positive-going wave (P1) is generated in the SCDH spheroid region after stimulation of cocultured DRG tissue (FIG. S3). Functional and pharmacological challenges confirmed the N1 peak to be presynaptic fiber-volley and the N2 peak to be a primary glutamatergic fEPSP while the less synchronous P1 peak likely represents secondary and higher order recurrent synaptic activity within the SCDH spheroid (FIG. 6-6). Finally, proof-of-concept, small-scale analgesic screening identified overlapping but distinct effects on microphysiological synaptic transmission induced by lidocaine, clonidine, and morphine that are consistent with in vivo descriptions of their distinct mechanisms of actions (FIG. 8).

In vivo, nociceptive signals originate in peripheral pain fibers and are transmitted unidirectionally to the CNS via synaptic transmission in the SCDH (53). Consistent with a single axis of meaningful information transfer, each large DRG soma extends a single large axon, and axons of many cells converge into relatively flat nerve tissue ultrastructure, reaching a vertical depth of only 50 μm despite several millimeters of horizontal growth (FIG. 2G,H). In contrast, the in vivo SCDH is a multilaminate structure in which information transfer occurs 3-dimensionally, both collaterally to neurons within the same layer and vertically to neurons of deeper and more superficial layers (54). Consistent with multiple axes of information transfer, small SCDH soma extend multiple, fine, ramified neuronal fibers that coalesce into a 3-dimensional neurite meshwork, reaching ˜300 μm in height despite very limited horizontal outgrowth (FIG. 2G,I). Emergent cellular and tissue morphology forms a tissue structure that permits both unidirectional information transfer between DRG and SCDH tissue and multidirectional information transfer within microphysiological SCDH tissue consistent with in vivo descriptions of their structure-function relationship.

This organized tissue ultrastructure facilitates emergence of the most valuable feature of microphysiological systems: in vitro reproduction of organized, complex, tissue-level physiology that is not possible with conventional culture methodology (55). Neurophysiology can be characterized, to an extent, using conventional dispersion culture combined with microelectrode array (MEA) or intracellular electrophysiological recording techniques. However, the physiology of a single neuron cultured in relative isolation on 2-dimensional substrates cannot approximate the complexity of, and is often much different than, the behavior of the same cell population within the context of a fully developed tissue (56-59). Here, we demonstrate organized tissue-level physiological interaction between DRG and SCDH cell populations in our defined MPS. This interaction occurs between distinct neurospheroid types separated by two millimeters and connected only by directed nerve fiber growth. This level of discrete tissue organization and connectivity exceeds that of assembloid MPS that consist of fused organoids grown in direct contact with each other or differentiated into distinct cell types in different parts of the same spheroid from the same pool of progenitors (34, 35). Critically, through both optogenetic and electrophysiological methods, we confirmed that concerted stimulation of DRG tissue results in a discrete, concerted physiological response in cocultured SCDH tissue through glutamatergic neurotransmission.

Optogenetic calcium imaging and field potential recording are complimentary approaches that together enable direct, rapid, highly informative, and clinically relevant physiological readouts appropriate for drug screening or preclinical research. The optogenetic approach affords ultimate control over tissue-type specific stimulation and physiological response recording, applied here to provide robust confirmation of the directionality of synaptic neurotransmission. Specific photostimulation of DRG tissue through viral CHR2-GFP expression robustly activated calcium influx in SCHD, specifically recorded through viral expression of CaMPARI-2, that was blocked after inhibition of glutamatergic neurotransmission. The optical clarity and ease of incorporating a variety of genetically modified neurons into this MPS affords extensive future opportunity for advanced optogenetic characterization of DRG-SCDH circuit physiology. With appropriately designed optogenetic tools, the physiology of individual neurons or neuronal subtypes can be directly evaluated within the context of overall circuit function, enabling detailed mechanistic description of analgesic actions in microphysiological DRG-SCDH circuits.

Extracellular field potential recording can be performed with higher throughput, enabling rapid identification of potential novel analgesics, and provides the greater time resolution necessary to differentiate the contribution of distinct underlying biological processes. We determined that glutamatergic input from the DRG tissue first generates an initial primary fEPSP in the SCDH spheroid, which then initiates secondary and higher-order recurrent synaptic transmission, which is limited in duration by GABAergic signaling. Dorsal horn field potentials recorded in ex vivo slice preparations from the rat spinal cord vary significantly based on recording lamina and stimulation site (60) and this MPS does not currently replicate the complexity of a fully developed rat spinal cord. However, both ex vivo rat dorsal horn and the microphysiological synaptic waveform are comprised of fast synaptic components followed by a longer, slower, positive deflection that are not observed after blockade of calcium influx or glutamatergic inhibition and modulated by GABAAR inhibition (61). We hypothesized that these common electrophysiological features shared by microphysiological DRG-SCDH cocultures and ex vivo rat spinal cord slices can be exploited to infer analgesic potential of novel therapeutics.

We tested this hypothesis by characterizing the effect of known analgesics (lidocaine, clonidine, and morphine) on synpatic transmission in this MPS. The analgesic properties of the local anesthetic, lidocaine, are primarily mediated by impairment of action potential propagation through peripheral pain fibers by enhancing the hyperpolarizing afterpotential, but is not thought to directly affect synaptic transmission (62). Similarly, in microphysiological DRG-SCDH coculture, lidocaine primarily reduced the sensitivity of the presynaptic fiber volley peak, N1 (FIG. 8G, FIG. S7). After controlling for the magnitude of N1, there were no direct effects of lidocaine on the synaptic components of the microphysiological waveform identified (FIG. 8D-F). The α2 adrenergic receptor agonist, clonidine, impairs action potential propagation through peripheral nerve fibers, but also inhibits voltage-gated calcium channels, impairing presynaptic neurotransmitter release in addition to desensitization of afferent pain fibers (62-64). The synaptic effects of clonidine predominate in microphysiological DRG-SCDH coculture. Synaptic components N2 and P1 were primarily desensitized after clonidine treatment (FIG. 8G). After controlling for N1 amplitude, there was a trending decrease the amplitude of the primary fEPSP, N2, and the higher-order recurrent synaptic transmission peak, P1, and a significant decrease in the early (but not late) phase of P1 (FIG. 8E,F). Thus, this MPS readily distinguished the electrophysiological profile of a local anesthetic that primarily targets desensitization of afferent pain fibers from an α2 adrenergic receptor agonist that primarily affects synaptic transmission.

In vivo, opioid receptors are found both presynaptically, on DRG-derived afferent nerve terminals, and post-synaptically on resident SCDH neurons (65). Activation of presynaptic opioid receptors has been shown to impair afferent DRG-derived mechanoreceptor synaptic input to the SCDH through inhibition of voltage-gated calcium channels (66), but is not thought to impair CAP propagation along the length of DRG-derived nociceptors. Opioid receptor-dependent inhibition of GABAergic interneurons is a well-established mechanism of opioid-induced disinhibition of excitatory neurons in both the brain and the spinal cord (31, 33). Morphine perfusion in microphysiological DRG-SCDH primarily desensitized the synaptic components N2 and P1 of the microphysiological waveform, similar to clonidine (FIG. 8G). After controlling for N1 amplitude, morphine significantly reduced the primary fEPSP and significantly reshaped the P1 peak by specifically increasing the late (but not early) phase (FIG. 8E,F). Impairment in primary fEPSP production is consistent with reduced presynaptic release of neurotransmitter while prolonged propagation of higher-order synaptic activity is consistent with disinhibition of SCDH circuits. The enhanced late phase of P1 strongly resembled the effect of direct GABAAR inhibition with bicuculline (FIG. 7C,F). Thus, it is likely that opioid receptor activation disinhibits microphysiological SCDH through inhibition of GABAergic neurotransmission similar to in vivo descriptions of opioid effects on the brain and spinal cord (31, 33). We hypothesize that screening potential novel analgesics for similar effects on synaptic transmission in this MPS will identify therapeutics with greater analgesic potential prior to exhaustive testing in animal models, thereby increasing the efficiency of analgesic development.

The microphysiological approach offers distinct advantages over conventional in vitro and in vivo model systems that may augment and streamline large-scale analgesic screening. While necessary to validate the physiological relevance of emerging analgesics and evaluate off-target effects, in vivo experimentation cannot be conducted with high enough throughput to evaluate all candidate compounds. Conversely, conventional 2-dimensional in vitro experimentation can be conducted at large scale but is unable to recapitulate tissue-level nerve function and may not accurately represent in vivo cellular neurophysiology (67-70). 3-dimensional in vitro model systems more closely approximate in vivo physiological function and may therefore more accurately identify novel compounds with analgesic potential. The results described here represent the first descriptions of analgesic-responsive, concerted, tissue-level DRG-SCDH synaptic transmission measured in vitro in a manner that is scalable for high-throughput experimentation. As many as 50 identical microphysiological cultures can be generated from a single litter of embryonic rats, each of which can be physiologically analyzed in a matter of minutes.

The application of microphysiological systems has exciting potential for basic research into the neurobiology of pain and efficient screening of novel analgesics. The use of human pluripotent stem cell (hPSC)-derived tissue in microphysiological systems has recently garnered considerable interest as it stands to eliminate interspecies differences that often confound translational research and enable patient-specific disease modeling and therapeutic intervention (20, 71). However, nearly everything we know about the neurobiology of pain has been described using preclinical animal models. Information obtained from hPSC-derived microphysiological systems cannot be properly contextualized within the literature without a deep understanding of emergent behavior of both hPSC and animal-derived cells in microphysiological systems (21). Here, we show how critical emergent behavior of embryonic rodent DRG and SCDH tissue in MPS enables tissue to spontaneously assemble into a neuronal circuit that recapitulates the critical aspects of in vivo DRG-SCDH circuit physiology necessary to profile the effects of distinct analgesics. Animal-derived cellular models such as these not only can be used immediately for drug screening or basic research but inform the design of analogous human MPS and provide a bridge of understanding to inform how novel human MPS relate to the vast body of research performed using animal models on which our basic understanding of neurophysiology is based.

### Materials and Methods

This study aimed to develop an in vitro microphysiological model of tissue-level synaptic transmission between peripheral nociceptors and dorsal spinal cord neurons and validate potential future use in analgesic screens. We first sought to identify an optimal ECM composition that promotes the emergence of the appropriate structure-function relationship between DRG and SCDH nerve tissue. Our lab has previously had success growing 3-dimensional nerve cultures in both gelatin methacrylate and Matrigel-based hydrogels and hypothesized based on previous reports (41-46) that modulation of gel stiffness could be used to direct neurite growth. Therefore, we designed gelatin methacrylate and Matrigel-based gel formulations that would result in ECM of various stiffness and compared morphology and physiology of cultures across gel formulations to identify the optimal ECM. We next sought to confirm that cultures adopted the robust, unidirectional DRG-SCDH tissue growth in the identified optimal ECM. Unidirectionality of nerve connectivity and information transfer is characteristic of in vivo DRG-SCDH circuits and we hypothesized that this tissue structure would also give rise to the desired unidirectional synaptic connectivity in microphysiological culture. To distinguish neurites emanating from different spheroids in the shared growth channel, select spheroids were infecting with GFP-expressing AAV prior to seeding in microphysiological culture. We then compared the relative extension of DRG-derived and SCDH-derived neurites into the shared growth channel and evaluated any evident cellular and tissue-level morphological differences. We next aimed to confirm that concerted, afferent DRG-SCDH, tissue-level synaptic function was present in microphysiological cultures under optimal ECM conditions. Multiple orthogonal optogenetic, electrophysiological, and pharmacological approaches were applied to selectively excite the DRG neuronal population and selectively inhibit neurotransmission to confirm that DRG excitation results in SCDH excitation through synaptic communication. Finally, we reasoned that any potential model of lower afferent nociceptive signaling would need to be responsible to common analgesics and we hypothesized that common analgesics with distinct underlying mechanisms of action would result in differential modulation of synaptic physiology in this microphysiological coculture. We identified lidocaine, clonidine, and morphine as three common analgesics with distinct underlying mechanisms. We applied these analgesics to our microphysiological cultures and quantified and compared consistent changes to the sensitivity and shape of the characteristic microphysiological synaptic waveform.

### Microphysiological Coculture System

All experiments were performed using a 3D dual-hydrogel MPS (FIG. 1). Fabrication methods have previously been extensively described and validated (36-40, 72, 73). Briefly, all DRG and SCDH nerve tissue was harvested from entire litters of embryonic day 15 (E15) rat embryos. All tissue was pooled by type, dissociated into single cell suspensions, and re-aggregated in spheroid microplates generating 30-50 spheroids of each type, identical in size and composition. Growth-restrictive outer-gel molds were patterned through digital projection lithography to define the shape of permissive growth area, spheroids were seeded in molds, and molds were filled with growth-permissive Matrigel. Spheroids extended neurites throughout the growth permissive inner gel forming microphysiological nerve tissue over the course of a three-week maturation period.

### Spheroid Formation

All animal handling and tissue harvesting procedures were performed according to guidelines set by the U.S. NIH and approved in advance by the Institutional Animal Care and Use Committee (IACUC) at Tulane University. DRG and SCDH tissue from an E15 rat litter were separately harvested, pooled, and digested in 0.25% trypsin in phosphate-buffered saline (PBS), pH 7.4, at 37° C. for 15 minutes. After digestion, tissue was pelleted at 500×g for five minutes, trypsin was removed, tissue was gently resuspended in growth media, dissociated through trituration, and passed through a 40 μM nylon mesh filter. DRG and SCDH cells were then separately seeded in 96-well ultra-low attachment spheroid microplates (Corning Inc., Corning, NY, USA) at a concentration of 45,000 DRG cells per well and 60,000 SCDH cells per well in growth media composed of Neurobasal Medium supplemented with 2% v/v B27 supplement, 1% v/v N2 supplement, 1% v/v GlutaMAX, 20 ng/mL nerve growth factor 2.5S native mouse protein, 10 ng/mL recombinant human/murine/rat brain derived neurotrophic factor (PeproTech, Cranbury, NJ, USA), 10 ng/mL recombinant human glial cell derived neurotrophic factor (PeproTech), and 1% v/v antibiotic/antimycotic solution (all from Thermo-Fisher Scientific, Waltham, MA unless otherwise noted). Microplates were centrifuged at 500×g for five minutes, and spheroids were aggregated after overnight incubation at 37° C. and 5% CO2.

### Dual-Hydrogel Coculture Fabrication

Outer gels were fabricated on Costar Transwell clear polyester membrane 6-well plate inserts (Thermo-Fisher) by irradiating a solution of 10% w/v polyethylene glycol dimethacrylate (Polysciences Inc., Warrington, PA, USA), 1.1 mM lithium phenyl-2,4,6-trimethylbenzoylphosphinate (LAP; Allevi, Philadelphia, PA, USA), and 0.0001% w/v TEMPO (Millipore-Sigma, St. Louis, MO, USA) in PBS (pH 7.4) with ultraviolet (UV) light patterned by a digital micromirror device (DMD, DLP 4500, Wintech Digital Systems Technology Crop., San Marcos, CA). The growth-restrictive polyethylene glycol gel will only polymerize when exposed to UV light, therefore the pattern of UV light projected by the DMD dictates the shape of the growth-restrictive mold. The DMD was used to pattern gel molds comprised of two bulbous regions, large enough for spheroid placement, connected by a long, thin channel that allows neurite growth between the spheroids (FIG. 1C). The desired spheroids were placed in the bulbs and the void was filled with a growth-permissive inner-gel solution, the inner-gel was cured, growth media was applied, and cultures matured for 17-21 days in growth medium at 37° C. and 5% CO2 prior to morphological or physiological analysis.

During initial growth-permissive gel stiffness optimization, both gelatin methacrylate and Matrigel-based inner gels of varying stiffness were prepared. Matrigel stiffness was modulated by increasing the concentration of Matrigel. Corning Matrigel hESC-qualified matrix (Thermo-Fisher) was diluted 1:2, 1:1, and 1:0 in growth media, applied to the inner-gel void, and was cured at 37° C. and 5% CO2 for 15 minutes prior to application of growth media. Gelatin methacrylate stiffness was modulated by increasing the concentration of 1-vinyl-2-pyrrolidinone in the gel precursor solution. 4% w/v gelatin methacrylate (Allevi) and 4 μg/mL laminin (Thermo-Fisher Scientific) were dissolved in 1.1 mM LAP and 1-vinyl-2-pyrrolidinone (Sigma) was added at 0%, 0.83%, 1.66%, or 2.5% v/v. The gel precursor solutions were added to the inner-gel void, gels were cured by irradiation with UV light from the DMD for 30 seconds, and growth media was immediately added to cured dual-hydrogel cultures. 50% Matrigel was determined to be the optimum ECM formulation and was used as the growth-permissive inner gel for all subsequent morphological and physiological analyses.

### Rheological Characterization of Gels

The viscoelastic properties of the gelatin methacrylate and Matrigel gel formulations were assessed using a shear rheometer (TA Instruments, AR2000, New Castle, DE). Circular samples (4 cm diameter) were prepared within a silicone mold on top of a Parafilm sheet (Bemis Company Inc., Neenah, WI). Gelatin methacrylate samples were exposed to 30 seconds of ultraviolet light (UV; 385 nm) to initiate crosslinking. Matrigel was allowed to self-assemble at 37 C for 30 minutes. For rheological testing, samples were loaded between the plate and the cone (1∘steel cone, 4 cm diameter, room temperature) before running a 0.1-10 Hz frequency sweep with a constant 4% strain. Storage (G′) and loss (G″) moduli were reported (n=3 per gel formulation). A one-way ANOVA was used to compare storage moduli.

### Nerve Viability Assay

Outgrowth from the spheroids in coculture was assessed using calcein AM from the LIVE/DEAD Viability kit (Thermo Fisher Scientific, Waltham, MA). Constructs were incubated in culture media containing the live stain for 30 minutes then imaged with the Nikon AZ100 (Tokyo, Japan) microscope at 0, 5, 10, 15, and 20 days in vitro (DIV) for dumbbell constructs and 0, 10, and 20 DIV for L-shaped constructs. The same construct was imaged at each timepoint to assess changes in outgrowth over time, data from DIV 10 are shown in FIG. S1.

### Neurite Outgrowth Tracing

The directionality of tissue growth was characterized by tracking the live migration of fluorescent neurites emanating from DRG and SCDH tissue over time. AAV9-Syn-GFP viral particles (Vigene Biosciences CV17001-AV9, Rockville, MD, USA) were incorporated during spheroid formation with a multiplicity of infection (MOI) of two million. The plasmid carried by this virus results in expression of GFP under control of the neuron-specific synapsin-I promoter. Infected spheroids were then washed in excess growth media and seeded in dual-hydrogel constructs. GFP-expressing DRG and SCDH spheroids were seeded both alone (monoculture) and in coculture with an uninfected spheroid of the other tissue type. Three replicate cultures of each of four conditions were analyzed including DRG(GFP) monocultures, SCDH(GFP) monocultures, DRG(GFP)-SCDH cocultures, and DRG-SCDH(GFP) cocultures. After 17 days of microphysiological nerve growth, GFP expression was imaged live on a fluorescent microscope (2× magnification with 5× optical zoom). The resulting images were then thresholded in ImageJ prior to quantification. All images were first processed by standard contrast limited adaptive histogram equalization (CLAHE) do reduce blur and a region of interest (ROI) was created to isolate the nerve growth channel. The auto-threshold function was applied to this ROI across all monoculture images to obtain an unbiased estimation of the appropriate threshold value. The estimated threshold value was averaged across all three DRG monocultures and separately averaged across all three SCDH monocultures. The average DRG monoculture threshold was then applied to all GFP-expressing DRG monocultures and cocultures and the average SCDH monoculture threshold value was applied to all GFP-expressing SCDH monocultures and cultures. A binary mask was then created from the thresholded image to assign each pixel as either supra- or sub-threshold. Finally, ROIs corresponding to sequential 0.8 mm bins spanning the entire construct were created and supra-threshold pixels were totaled in each bin to obtain a total above-threshold pixel count at discrete, increasing distances from GFP-expressing spheroids.

### Microphysiological Nerve Morphology

A batch of DRG and SCDH spheroids were generated and 20% of the spheroids were infected with AAV9-Syn-GFP with an MOI of two million to reduce the proportion of infected neurons and enable visualization of individual neurons. Spheroids were then washed and cocultured in dual-hydrogel constructs for 17 days. Live constructs were then imaged with 3D confocal microscopy on a Nikon confocal microscope to analyze both cellular and tissue-level morphology. For gross tissue morphology, three adjacent z-stacks encompassing the entire vertical growth of the culture were imaged at 10× magnification, stitched together, 3-dimensionally rendered with alpha blending, and depth coded in Nikon Elements software to produce a 3D map of culture thickness. Z-stacks encompassing the DRG and SCDH were also separately imaged at 20× for morphological analysis of individual neurons. Maximum intensity projections through select 20 μm vertical sections of the z-stacks were generated to highlight individual neuronal soma and proximal neurite projections. To calculate average somal size, the diameter of all soma with distinct borders was measured throughout the z-stack encompassing three DRG and SCDH spheroid regions from three independent cultures. The average somal diameter was calculated for each spheroid resulting in an “n” of 3 for statistical analysis.

### Preparation and Imaging of Whole-Construct Immunofluorescent Analysis

Mature constructs were removed from the incubator and washed 3×5 minutes with PBS to remove growth media from the dual-hydrogel scaffolds. Constructs were then fixed with ice-cold 4% paraformaldehyde for 20 minutes and washed 3×10 minutes with ice-cold PBS to remove excess fixative. After immunostaining, cultures were mounted in ProLong Glass antifade mounting media with NucBlue DNA stain on a microscope slide and covered with a coverslip. Three-dimensional antibody staining was visualized on an Olympus confocal microscope. Three adjacent image stacks were generated at 10× magnification and stitched together in cellSens to encompass the entire nerve construct. Images presented are maximum intensity projections through the resulting stitched image stack.

### Preparation and Imaging of Immunofluorescent Staining of Cryosections

Mature constructs were fixed with 4% paraformaldehyde for 30 minutes at 4° C. The solution was removed and constructs were washed 3×5 min with ice-cold PBS to remove excess fixative. Constructs were then sequentially submerged for 24 hours in 15% and 30% sucrose solution in PBS at 4° C. Sucrose solution was removed, and wells were filled with optimal cutting temperature compound (Thermo-Fisher Scientific) and placed in a dry ice/isopentane bath for five minutes. After freezing the construct, they were stored at −80° C. until cryosectioning. Constructs were cryosectioned perpendicular to the axis of neurite growth creating 10-20 μm thick slices which were transferred onto positively charged microscope slides. Sides were stored at −30° C. until immunostaining. After immunostaining, sections were mounted in ProLong Glass antifade mounting media with NucBlue DNA stain on a microscope slide and covered with a coverslip. Sections were imaged in cellSens software on an Olympus confocal microscope at 40× magnification and 3× digital zoom.

Images were then thresholded in ImageJ for colocalization analysis. Images representing each stain in the SCDH were individually opened and the threshold was increased to drop out faint background staining. Images were then converted to a binary mask (1=above, 0=below threshold) and the total number above-threshold pixels in the image were counted to quantify total staining for each antibody. Binary images representing individual channels were then multiplied together in a pixel-wise fashion to generate a new binary colocalization map (1=colocalization, 0=no colocalization). Total number of colocalized pixels were divided by the total number of above-threshold pixels in the individual channel images to calculate percent colocalization. To ensure consistency in the distribution of staining intensities include in the analysis, the thresholds identified in SCDH images were then applied to the analogous images obtained from DRG spheroid cryosections and the colocalization analysis was repeated.

### Immunofluorescent Staining of Whole Constructs and Cryosections

Tissue was blocked and permeabilized in 5% goat serum and 0.3% Triton-X-100 in PBS for one hour, incubated with primary antibodies overnight in 5% goat serum and 0.3% Triton-X-100 in PBS at 4° C., washed 3×10 minutes with 0.3% Triton-X-100 in PBS, incubated with secondary antibodies overnight in 5% goat serum in PBS at 4° C., and washed 3×10 minutes in PBS. All steps were performed at room temperature with gentle agitation unless otherwise noted. Primary antibodies and dilutions included mouse anti alpha-III-tubulin (B3T) at 1:1000 (Abcam, ab78078), chicken anti microtubule-associated protein 2 (MAP2) at 1:5000 (Novus Biologicals, NB300-213), rabbit anti peripherin at 1:1000 (Abcam, ab1530), rabbit anti vesicular GABA transporter (vGAT) at 1:1000 (Novus, NBP2-20857), rabbit anti synapsin-I at 1:500 (Abcam, ab64581), mouse anti-PSD95 (NeuroMab K28/43), and mouse anti CGRP at 1:500 (Abcam ab81887). Secondary antibodies included Alexa Fluor 594 goat anti mouse at 1:1000 (Abcam, ab150116), Alexa Fluor 488 goat anti rabbit at 1:1000 (Abcam, ab150077), and Alexa Fluor 647 goat anti chicken at 1:1000 (Abcam, ab150171).

### Optogenetic Methods

AAV1-hSyn-NES-his-CaMPARI2-WPRE-SV40 was a gift from Eric Schreiter (Addgene viral prep #101060-AAV1; http://n2t.net/addgene:101060; RRID:Addgene_101060) and AAV8-Syn-CHR2(H134R)-GFP was a gift from Edward Boyden (Addgene viral prep #58880-AAV8; http://n2t.net/addgene:58880; RRID:Addgene_5880). Viral particles of both types were added during spheroid formation with a MOI of 200,000. Cultures were seeded and matured for three weeks. Baseline red fluorescence was imaged with both three and ten second exposures prior to photostimulation using a Nikon AZ100 fluorescent microscope (2× magnification plus 5× or 8× digital zoom). All constructs across all experiments received an identical photostimulation protocol, during which the entire construct was illuminated for three seconds with 488 nm light focused through the fluorescent microscope (2× magnification, 5× digital zoom) to activate the CHR2-GFP fusion protein and 405 nm light was simultaneously applied with a 50 mW laser emitting diode focused on the spheroid region of interest to provide CaMPARI-2 photoconversion light. Post-photostimulation red fluorescence was again imaged with methods identical to baseline imaging. The integrated pixel intensity for a region of interest encompassing each spheroid of interest was quantified in Fiji (ImageJ) and post-photostimulation intensity was normalized to pre stimulation intensity to obtain a final value for fold change in integrated pixel intensity.

### Microphysiological Field Potential Recording

For all electrophysiological experiments, mature constructs were submerged in room-temperature artificial cerebrospinal fluid (aCSF) bath composed of 170 mM NaCl, 7 mM KCl, 37 mM NaHCO₃, 0.91 mM Na2HPO4·7H2O, 14 mM D+glucose, 4 mM MgSO4, and 2 mM CaCl2) in deionized water and continuously bubbled with 95% O2 and 5% CO2. A concentric bipolar stimulating electrode (FHC Inc., Bowdoin, ME, USA) was inserted into the nerve tissue at the desired stimulation site. The stimulus-response protocol was executed with LabChart Software (AD Instruments, Colorado Springs, CO, USA) including voltage steps with 0, 1, 2, 3, 4, 5, 6, 7, 8, 9, 10, 15, 20, 30, and 40-volt amplitudes. Resulting field potentials were recorded by a platinum wire (A-M Systems, Sequim, WA), inserted into a pulled-glass micropipette filled with aCSF with resistance adjusted to 1-2 MG. The tip of the glass recording pipette was inserted into nerve tissue at the desired recording site. Bioelectric signals were amplified with a Model 3000 AC/DC Differential Amplifier (A-M systems, Sequim, WA) set at 100× gain and 0.1 Hz high pass and 3 kHz low pass filtering, electrical interference was removed with the Hum Bug Noise Eliminator (Quest Scientific, North Vancouver, Canada), traces were digitized with the Powerlab analog-to-digital converter (AD Instruments), recorded in LabChart, and exported to Igor Pro (v. 8, WaveMetrics Inc., Lake Oswego, OR, USA) for quantitative analysis. The ten replicate traces obtained at each voltage step were averaged into a single trace representative of each step for analysis.

Field potential production was initially characterized in monocultures. DRG and SCDH monocultures were stimulated in the nerve growth region (concentrated nerve fibers) while recording in the spheroid region (concentrated neuronal soma) and stimulated in the spheroid region while recording in the nerve growth region (FIG. S3A,B,D,E). When recording in cocultures, one spheroid was electrically stimulated while resulting field potentials were recorded in the adjacent spheroid region (FIG. S3C,F,G). Additional constructs were generated that permitted bidirectional growth of DRG but not SCDH spheroid tissue and the waveform produced in the SCDH after short, medium, and long-distance stimulations was recorded and compared to infer the potential contribution of direct electrical activation of SCDH tissue to the DRG-SCDH synaptic waveform (FIG. S3H,I).

### Synaptic Fatigue

In DRG-SCDH cocultures, 20 V, 200 μsec square-wave stimulations were delivered to DRG tissue sequentially at 10 Hz, 20 Hz, and 25 Hz for one second while recording waveform production in the SCDH. The amplitude of each waveform component (N1, N2, and P1) was measured in Igor Pro for each response to the first ten successive stimulations at each frequency. The amplitude of each component at each successive stimulation was then subtracted from and normalized to its baseline amplitude (after the first stimulation in that train) to calculate the percent change from baseline of each peak after each stimulation in each frequency train.

### Pharmacological Challenges

DRG-SCDH cocultures were further challenged through specific pharmacological inhibition of glutamatergic and GABAergic neurotransmission to confirm the synaptic nature of the DRG-SCDH waveform. Constructs were removed from culture media and equilibrated in aCSF for 30 minutes and a baseline stimulus-response curve was performed. Cultures were then perfused for ten minutes with aCSF containing various pharmacological treatments and the stimulus-response curve was repeated and compared to the baseline data to isolate drug-dependent effects. aCSF composed of 1 mM ethylenediaminetetraacetic acid (EDTA, Millipore-Sigma) and 0 mM CaCl2) was perfused to inhibit all synaptic transmission. aCSF containing 10 μM 6-Cyano-7-nitroquinoxaline-2,3-dione disodium salt hydrate (CNQX, Millipore-Sigma) was perfused to inhibit AMPA/Kainate receptor-dependent glutamatergic neurotransmission. 100 μM (+)-bicuculline (Millipore-Sigma) was perfused to inhibit 7-aminobutyric acid (GABA) receptor type A (GABAAR)-dependent GABAergic neurotransmission. 1 μM tetrodotoxin (Abcam) was perfused to inhibit all voltage-gated sodium channel-dependent CAP conduction. CNQX, bicuculline, and tetrodotoxin were dissolved in DMSO at 1000× and diluted 1:1000 in aCSF when used. DMSO concentrations were maintained at 0.1% throughout all pharmacological experiments.

The waveform corresponding to each voltage step under baseline and drug-treated conditions was quantitatively analyzed with a custom algorithm written in Igor Pro that identifies the location of the N1 peak, the N2 peak, and the P1 peak. Accurate peak selection was confirmed by a scorer blind to the treatment groups. The maximum amplitude of each peak, the latency to each peak, the difference in amplitudes between each peak, the difference in latencies between each peak, the integrated area under the curve (AUC) between N2 and P1 (P1-early), the integrated AUC between P1 and the end of the recording (P1-late), and the integrated area from N2 to the end of the trace (P1-total) were automatically calculated for each trace. There was no indication of changes in presynaptic sensitivity following treatment with EDTA, CNQX, or bicuculline; therefore maximal (40V) stimulation traces were directly compared between baseline and drug-treated stimulus response curves.

### Comparison of Analgesics

Three commonly used analgesic compounds of different classes were applied to DRG-SCDH cocultures to characterize their effects on microphysiological afferent pain signaling. A local anesthetic, (lidocaine), an α2 agonist (clonidine), and the prototypical opioid analgesic (morphine) were perfused in aCSF at increasing concentration to identify lowest effective dose. DRG-SCDH constructs were again equilibrated in aCSF for 30 minutes to achieve a stable baseline. Lidocaine (Millipore-Sigma) was first dissolved in DMSO and diluted into aCSF to a final concentration of 10 μM, 100 μM, 500 μM, and 1 mM. Clonidine (Millipore-Sigma) was dissolved directly in aCSF (with 0.1% DMSO) at 100 μM and diluted to concentrations of 100 nM, 1 μM, 10 μM, and 100 μM. Morphine sulfate (provided by the NIH National Institute on Drug Abuse) was dissolved directly in aCSF (with 0.1% DMSO) and diluted to concentrations of 1 μM, 10 μM, 100 μM, and 500 μM. A stimulus-response curve was performed after application of each dose of each drug and compared to baseline recordings to isolate drug-dependent effects (FIG. S5). A washout was then performed to ensure the reversibility of the drug-dependent effects (FIG. S6).

The lowest effective doses for each drug were then replicated four times for in-depth quantification and statistical analysis. Constructs were placed on the rig and equilibrated in aCSF for 30 minutes and a stimulus-response curve was obtained at baseline. A sham treatment was then performed in which the entire recording bath was drained and was replaced with aCSF containing vehicle (0.1% DMSO), the construct was incubated for ten minutes, and a second stimulus-response curve was obtained. The entire bath was drained again and was replaced with aCSF containing the analgesic treatment, then the construct was incubated for ten minutes, and a third stimulus-response curve was obtained.

The waveform corresponding to each voltage step under baseline, sham, and analgesic-treated conditions was quantitatively analyzed with a custom algorithm written in Igor Pro that identifies the location of the N1 peak, the N2 peak, and the P1 peak. Accurate peak selection was confirmed by a scorer blind to the treatment groups. The maximum amplitude of each peak, the latency to each peak, the difference in amplitudes between each peak, the difference in latencies between each peak, the integrated area under the curve (AUC) between N2 and P1 (P1-early), the integrated AUC between P1 and the end of the recording (P1-late), and the integrated area from N2 to the end of the trace (P1-total) were automatically calculated for each trace.

After initial analysis, it was apparent that analgesic treatments affected the presynaptic fiber-volley peak (N1) in addition to the synaptic components N2 and P1. A second analysis was performed to control for apparent analgesic-induced differences in afferent input sensitivity. The waveform produced after 40 V stimulation of analgesic treated constructs was matched with the lower-intensity sham waveform with the most similar N1 amplitude. This comparison aligns traces with the most similar apparent strength of afferent input and any remaining differences are more likely direct effects on synaptic transmission. Finally, analgesic-induced differences in the sensitivity of the three different waveform components were directly calculated. The maximal amplitude of N1, N2, and P1 were obtained from the 40V analgesic-treated traces. The sham treated traces that resulted in the most similar amplitudes for each of these components were then identified.

### Statistical Analysis

The GFP-expressing neurite tracing experiment was designed to separately compare neurite outgrowth from DRG and SCDH monocultures to growth of these same tissues in DRG-SCDH coculture. Two 2-way mixed model ANOVAs with the between-subjects factor of “culture type” (levels include “monoculture” and “coculture”) and the within-subjects factor of “location” (levels include bins of increasing distances from GFP-expressing spheroid in 0.8 mm increments) were used to identify significant differences in GFP-expressing neurite density over increasing distance from GFP-expressing DRG and SCDH spheroid centers. Following identification of significant “culture type” by “location” interaction, LSD post-hoc tests were performed to identify significant differences between levels of “culture type” at each level of “location”.

A significant difference in the average somal size between DRG and SCDH neurons was tested with a Student's t-test. The diameters of all clearly distinguishable neuronal soma were measured across three different DRG and SCDH spheroids. The average diameter of all soma within each construct was calculated and these six average (three DRG and three SCDH) values were compared with a Student's t-test.

The optogenetic experiments were conducted with between-samples designs. The fold change in red fluorescence before and after photostimulation was compared between control cultures and groups of cultures that received different independent treatments. Thus, the difference in fold change between control and treatment groups was compared with an unpaired t-test in four separate experiments. First, three DRG(CaMPARI-2) cultures were compared to three DRG(CHR2-GFP+CaMPARI-2) cultures. Images were collected with a three second exposure and 2× magnification with 8× zoom (FIG. 4A,B). Second, eight identical DRG(CaMPARI-2) constructs were split into two groups which were treated for five minutes with either 10 μM capsaicin or vehicle alone (0.1% DMSO) prior to photostimulation to record capsaicin-induced calcium currents. Images were collected with a ten second exposure at 2× magnification with 8× zoom (FIG. 4A,C). Third, three DRG(WT)-SCDH(CaMPARI-2) were compared to three DRG(CHR2-GFP)-SCDH(CaMPARI-2). Images were collected with a three second exposure at 2× magnification with 5× zoom (FIG. 4D,E). Finally, eight identical DRG(CHR2-GFP)-SCDH(CaMPARI-2) cultures were split into two groups which were treated with either 200 uM CNQX or vehicle alone (0.28% DMSO) for 15 minutes prior to photostimulation to inhibit glutamatergic neurotransmission. Images were collected with a three second exposure and 2× magnification with 8× zoom (FIG. 4F,G).

Electrophysiological experiments were conducted according to within-samples designs. Each described metric was measured before and after treatment from electrophysiological waveforms derived from the same construct. To account for between-construct differences in the absolute magnitude of the waveform (independent of treatment), the value of the metric after treatment was subtracted from its value before treatment to obtain a “change in metric” value. The change in each metric was then compared with a one-sample t-test to a value of zero, which would be indicative of no change. The synaptic fatigue experiment was replicated in three independent cultures. The percent change from baseline was averaged across experiments and significant changes in each described metric were identified with a one-sample t-test vs 0% (no change) after the tenth stimulation (FIG. 6B). Pharmacological inhibition of neurotransmission was replicated four or five times. Four independent constructs were treated with EDTA, five constructs were treated with CNQX, and four constructs were treated with bicuculine and the percent change from baseline of each described metric was averaged across constructs and significant changes were identified with a one sample t-test vs 0% (FIG. 7D-F). Each analgesic was applied to four independent cultures. The change in each of these values between baseline and sham treatments, and between sham and analgesic treatments at each stimulation intensity was averaged across constructs and a one sample t-test vs 0 (no change) was performed to identify significant sham or analgesic treatment induced changes at each voltage (FIG. S7). To control for apparent differences in presynaptic desensitization, differences in each described metric between the 40V analgesic-treated waveform and the N1-matched sham waveform were averaged across constructs and significant differences were determined with a one sample t-test vs 0 (FIG. 8D-F). Finally, the percent change in stimulus intensity required to evoke this response of similar amplitude was averaged across constructs for each waveform component and statistical significance was determined with a one sample t-test vs 0% (FIG. 8G).

TABLE S1
Detailed statistical analysis of GFP-expressing
neurite tracing study shown in FIG. 2A-F.

| | | SCDH | | | DRG | | |
|---|---|---|---|---|---|---|---|
| | | F | p | df | F | p | df |
| Main | Culture | 13.76 | 0.0207 | 1 | 0.9611 | 0.3824 | 1 |
| Effect | Type | | | | | | |
| | Location | 84.18 | <0.0001 | 25 | 14.29 | 0.0005 | 26 |

| | | | | | | |
|---|---|---|---|---|---|---|
| Interaction | 4.987 | <0.0001 | 25 | 2.461 | 0.0007 | 26 |

LSD Post Hoc Tests

| Distance from SCDH | p | Distance from DRG | p |
|---|---|---|---|
| −1.68 | 0.2242 | −0.16 | 0.0796 |
| −1.5 | 0.4405 | −0.06 | 0.3116 |
| −1.52 | 0.2812 | 0 | 0.4093 |
| −1.44 | 0.313 | 0.08 | 0.3858 |
| −1.35 | 0.5252 | 0.16 | 0.0615 |
| −1.28 | 0.649 | 0.24 | 0.2051 |
| −1.2 | 0.8656 | 0.32 | 0.9218 |
| −1.12 | 0.1516 | 0.4 | 0.8851 |
| −1.04 | 0.0375 | 0.48 | 0.6431 |
| −0.96 | 0.0271 | 0.56 | 0.8298 |
| −0.88 | 0.0311 | 0.64 | 0.7502 |
| −0.8 | 0.0239 | 0.72 | 0.8044 |
| −0.72 | 0.0082 | 0.8 | 0.4109 |
| −0.64 | 0.0086 | 0.88 | 0.2824 |
| −0.56 | 0.0002 | 0.96 | 0.2656 |
| −0.48 | 0.0057 | 1.04 | 0.205 |
| −0.4 | 0.1475 | 1.12 | 0.774 |
| −0.32 | 0.9745 | 1.2 | 0.6555 |
| −0.24 | 0.5352 | 1.28 | 0.6175 |
| −0.15 | 0.3584 | 1.36 | 0.3276 |
| −0.08 | 0.4226 | 1.44 | 0.0107 |
| 0 | 0.4226 | 1.52 | 0.0873 |
| 0.08 | 0.4226 | 1.6 | 0.2088 |
| 0.16 | 0.4022 | 1.68 | 0.289 |
| 0.24 | 0.178 | 1.76 | 0.3192 |
| 0.32 | 0.2252 | 1.84 | 0.4218 |
| | | 1.92 | 0.2543 |

Analysis of GFP-expressing DRG tissue identified a significant main effect of “culture type” (F=13.76, p=0.0207, df=1), a significant main effect of“location” (F=84.18, p<0.0001, df=25), and a significant interaction between “culture type” and “location” (F=4.987, p<0.0001, df=25). Analysis of GFP-expressing SCDH tissue also identified a significant main effect of“location” (F=14.29, p=0.0005, df=26) and a significant interaction between “culture type” and “location” (F=2.461, p=0.0007, df=26), but did not identify a main effect of “culture type”. LSD post-hoc tests were employed to describe the source of these significant differences between bin locations across construct types. DRG tissue in co-cultures showed little significant difference in neurite outgrowth except at a single location approximately 1.5 mm distal from the DRG spheroid (p=0.0107), consistent with the front side of the unlabeled SCDH spheroid (FIG. 2E). In contrast, compared to SCDH monocultures, GFP-expressing SCDH tissue in co-cultures showed a consistently significant decrease in neurite growth between 0.5 and 1 mm distal from the SCDH spheroid, corresponding to the shared growth channel (FIG. 2F).

TABLE S2
Full p-value summary of N1-matched analgesic waveform analysis.

| | Lidocaine | Clonidine | Morphine |
|---|---|---|---|
| N1 Latency | 9.0203 | 0.8378 | 0.9494 |
| N1 Amplitude | 0.731 | 0.2258 | 0.5587 |
| N2 Latency | 9.0372 | 0.8695 | 0.9212 |
| N2 Amplitude | 0.1588 | [symbol] .0799 | 0.9198 |
| P1 Latency | 0.7 [symbol] 96 | [symbol] .1364 | [symbol] |
| P1 Amplitude | [symbol] | [symbol] | 0.243 |
| P1 Early AUC | 0.9854 | [symbol] | [symbol] |
| P1 Late AUC | 0.5329 | [symbol] | [symbol] |
| P1 Total AUC | 0.5829 | [symbol] .2623 | 0.0218 |
| N1 Sensitivity | 0.0022 | 0.2152 | 0.117 |
| N2 Sensitivity | 0.0 [symbol] 12 | 0.0163 | 0.0034 |
| P1 Sensitivity | [symbol] | [symbol] | [symbol] |

[symbol]

indicates data missing or illegible when filed

N1-matching was performed to align maximum amplitude analgesic-treated traces with pretreatment traces with the most similar N1 amplitude evoked by lower intensity stimulation. Differences in N1, N2, and P1 latency and amplitude as well as P1 as well as early, late, and total integrated area under the curve (AUC) of P1 between pretreatment and analgesic-treated traces were calculated and significant changes were determined with a one sample t-test vs 0 (no change). Exact p-values are presented, and significant (or very nearly significant) values are highlighted in bold font and correspond to the analysis shown in FIG. 8D-G.

### Example 2

### Human Methods

Generation o±3D Spheroid Cultures. Cryogenically preserved human stem-cell derived spinal cord dorsal horn (SCDH) precursor cells were a kind gift from the Randolph Ashton. Cells were rapidly thawed and diluted 1:4 in Neurobasal Medium (Thermo-Fisher Scientific, Waltham, MA). Cells were centrifuged at 300 g for 4 minutes and the supernatant was removed. Cells were then resuspended in neuronal differentiation media comprised of Neurobasal Medium supplemented with 2% v/v B27 supplement (Thermo-Fisher), 1% v/v N2 supplement (Thermo-Fisher), 1% v/v GlutaMAX (Thermo-Fisher), 20 ng/mL human nerve growth factor, 10 ng/mL recombinant human/murine/rat brain derived neurotrophic factor, 10 ng/mL recombinant human glial cell derived neurotrophic factor, 10 ng/mL recombinant human NT-3 (all growth factors from PeproTech, Cranbury, NJ, USA), 1 uM dibutyrl-cAMP (Millipore-Sigma, St. Louis, MO, USA), 10 uM Y-27632 (Abcam, Waltham, MA, USA), 10 uM DAPT (Tocris, Minneapolis, MN, USA), and 1% v/v antibiotic/antimycotic solution (Thermo-Fisher). Cells were plated at 500,000 cells/cm² on culture well plates coated with Matrigel (Corning) diluted at 1:100 for six days in neuronal differentiation media changed every-other-day to force terminal neuron differentiation. Cryogenically preserved human stem cell-derived nociceptors were purchased from Anatomic, Inc (Minneapolis, MN, USA). Cells were rapidly thawed, diluted 1:4 in Neurobasal Medium, centrifuged at 300 g for 4 minutes, and the supernatant was removed. Nociceptors were then resuspended in human culture growth media (identical to neuronal differentiation media but lacking DAPT and Y-27632) plated at 80,000 cells/cm² in well plates coated with Matrigel diluted at 1:100, allowed to adhere overnight, and washed to remove any dead cell debris. Nociceptor and terminally differentiated SCDH cell cultures were lifted by incubation in Accutase at 37° C. for 15 minutes. The cell suspensions were collected, centrifuged at 300 g for 4 minutes, the supernatant was removed, and both cell types were resuspended in culture growth media. Cells were then seeded in 96-well ultra-low attachment spheroid microplates (Corning) at a concentration of 20,000 cells per well and microplates were centrifuged at 500 g for 5 minutes. 3D neural spheroids were allowed to aggregate for 48 hours at 37° C. and 5% CO2.

Micropatterning Microphysiological Nerve Cocultures. Fabrication and validation of 3D peripheral nerve cultures has previously been described extensively (Bowser and Moore 2019; Curley and Moore 2011; Huval et al. 2015; Khoshakhlagh et al. 2018; Kramer et al. 2020; Sharma et al. 2019). Outer-gels were fabricated by irradiating a solution of 10% w/v polyethylene glycol dimethacrylate (PEG, Polysciences Inc., Warrington, PA, USA), 1.1 mM lithium phenyl-2,4,6-trimethylbenzoylphosphinate (LAP; Allevi, Philadelphia, PA, USA), and 0.0001% w/v TEMPO (Millipore-Sigma, St. Louis, MO, USA) in phosphate-buffered saline (PBS, pH 7.4) with ultraviolet (UV) light patterned to create an inner void that is 2 mm long and 0.4 mm wide that is continuous with 0.75 mm diameter circular bulbs on either end for spheroid placement, surrounded by a growth-restrictive PEG mold. A nociceptor spheroid was placed in the bulb at one end of each outer-gel mold with a pipette, a spinal cord dorsal horn spheroid was placed on in the bulb on the opposite end. The void was then filled with an inner-gel solution composed of Matrigel (Corning Inc., Corning, NY, USA) diluted 1:1 with culture growth media, and the construct was incubated for 15 minutes at 37 C to cure the Matrigel. Culture media was added underneath the transwell membrane for each construct and cultures were returned to the incubator for maturation.

Maturation of Microphysiological Nerve Cultures. Assembled dual-hydrogel DRG-spheroid cultures were then incubated for 40-240 days in human culture growth medium, changed on Monday, Wednesday, and Friday, during which neurites extend from the nociceptor spheroid along the permissive growth channel to innervate the SCDH spheroid at the opposite end of the channel.

Evoked Response Field Potential Recording. At the time of recording, mature dual-hydrogel human cocultures were manually transferred from the incubator to the electrophysiology apparatus. The culture media was removed and cultures were continuously bathed with artificial cerebrospinal fluid (ACSF) composed of 170 mM NaCl, 7 mM KCl, 37 mM NaHCO₃, 0.91 mM Na2HPO4·7H2O, 14 mM D+glucose, 4 mM MgSO4, and 2 mM CaCl2) in deionized water and continuously bubbled with 95% O₂ and 5% CO₂. A platinum and glass recording electrode (1 μMΩ resistance) was lowered into the SCDH spheroid region and a concentric bipolar platinum stimulating electrode was placed in the innervating nociceptor spheroid. Bipolar 40 V, 200 μsec stimuli were delivered once per minute to the middle of the nociceptor spheroid for the duration of the experiment. Resulting CAP propagation was recorded in the SCDH spheroid region by the external glass and platinum field recording electrode for ten seconds following each stimulation. Experimental drugs were applied through the perfusing ACSF at defined timepoints to record the evolution of the waveform across each experiment.

Spontaneous Field potential Recording. Experiments describing the spontaneous activity in these cultures were identical to the evoked response field potential recording methods, except that the stimulator delivered a 0V stimulus instead of a 40V stimulus.

### Summary of the Differences Between Human and Rats

An extensive stimulation protocol was used in rat tissue to generate a full stimulus-response curve. Rat DRG spheroids were stimulated at 0.5 Hz with increasing intensity from 0 V to the maximum 40 V stimulation. This stimulation protocol was found to be not feasible for young hPSC-derived nerve cultures as 0.5 Hz stimulation was found to induce apparent long term depression of the circuit. Initially, a coordinated response of a given magnitude could be recorded in the SCDH spheroid after stimulation of the adjacent nociceptor spheroid. However, after 10 stimulations at 0.5 Hz it was no longer possible to record a coordinated response in the SCDH spheroid to a coordinated stimulation of the nociceptor spheroid 2 mm distal. It was found that increasing the inter-stimulus interval to 60 seconds minimized the amount of apparent long-term depression after the same number of stimulations. Therefore, hPSC-derived nerve cultures were stimulated with the maximal 40 V stimulation at a frequency of one stimulation every minute to ensure that the experiments were free of long-term depression.

### Maturation of the Nerve Model

The pattern of bioelectric data recorded from these cultures are a function of the cellular composition of each spheroid, the gross geometry of the hydrogel system, the specific geometry of physical synaptic connections formed within and between the nociceptor and dorsal spinal cord tissues, and the maturity of the tissue. Thus, the shape and patterns of the recorded electrophysiological waveforms are unique and specific to this particular dual-hydrogel system with strategically-directed nerve bundle growth, combination and placement of spheroids, and the length of time the functional synaptic connections have been given to mature. As the cultures continue to mature, individual types of cells become more or less excitable, leading to spontaneous bursts of activity with the cultures. This, in turn, leads to plasticity of the functional synaptic connections within and between spheroids continuously, with some connections strengthening and others weakening, generating new waveform shapes and patterns of activity. These changes in the levels of spontaneous activity and patterns of functional synaptic connections create different patterns and geometries of the bioelectric activity that is observed at the recording electrode, generating electrophysiological waveforms with different shapes and timing than younger cultures. Although the waveforms recorded in older cultures have different shapes and patterns than those of young cultures, both provide important information about the underlying biology and the response of these tissues to applications of biologically active drugs and potential novel analgesics.

### Bioelectric Waveforms in Young Cultures

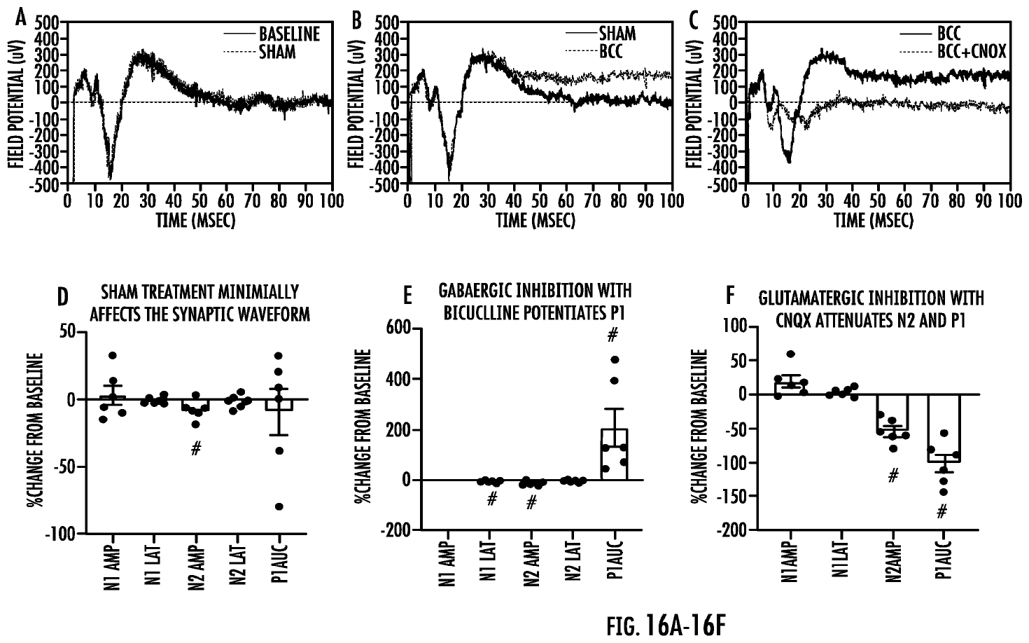

The shape and behavior of younger embryonic rat cultures is described extensively in Pollard et al. 2021. Similar waveforms were observed in young human pluripotent stem cell-derived nerve cultures after 6-8 weeks of maturation. This waveform consists of early, negative-going spike in activity (analogous to N1), a later, negative going spike in activity (analogous to N2), and an even later, positive-going wave of activity (analogous to P1). (FIG. 16A). Consistent with the young rat cultures, application of the GABAa receptor antagonist, bicuculline (BCC), prolongs the period of synaptically evoked activity in dorsal spinal cord tissue following stimulation of the afferent nociceptor spheroid (FIG. 16B,E) and application of the AMPA type glutamatergic receptor antagonist CNQX blocks a large portion of the synaptically evoked activity (FIG. 16C,F). This indicates that the same basic neuronal functions of glutamatergic excitation and GABAergic inhibition shape the pattern of electrophysiological activity in cocultures of hPSC-derived nociceptors and dorsal spinal cord cells and embryonic rat DRG-SCDH cocultures.

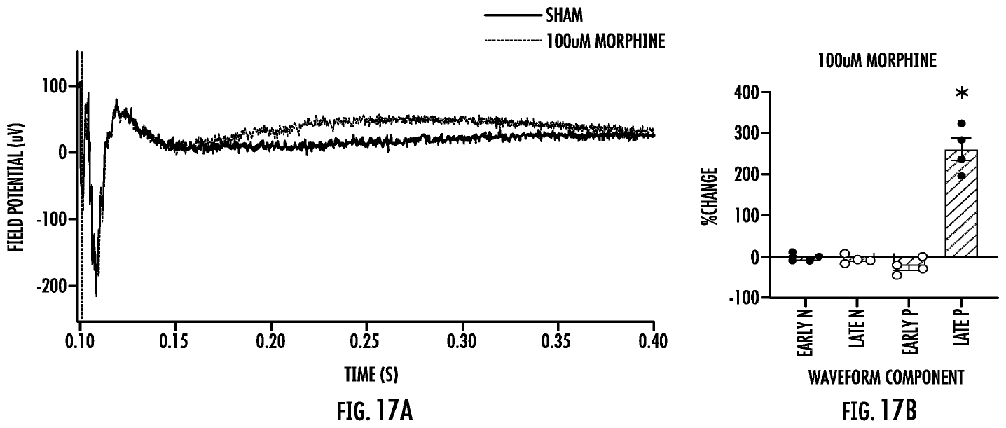

Additionally, activation of opioid receptors in hPSC-derived nociceptor-dorsal spinal cord cocultures produces similar effects to what was described previously in embryonic rat DRG-SCDH cocultures. Application of 100 uM morphine prolongs the late, positive-going portion of the synaptically evoked waveform in dorsal spinal cord spheroids upon stimulation of afferent nociceptor tissue (FIG. 17A,B) in a manner similar to bicuculline.

Thus, these human stem cell-derived cocultures matured for a relatively short period of time (6-8 weeks) in our specific microphysiological system can be used to infer the analgesic potential of novel pharmaceuticals through methodology similar to that described in Pollard et al. 2021.

### Example Experiments

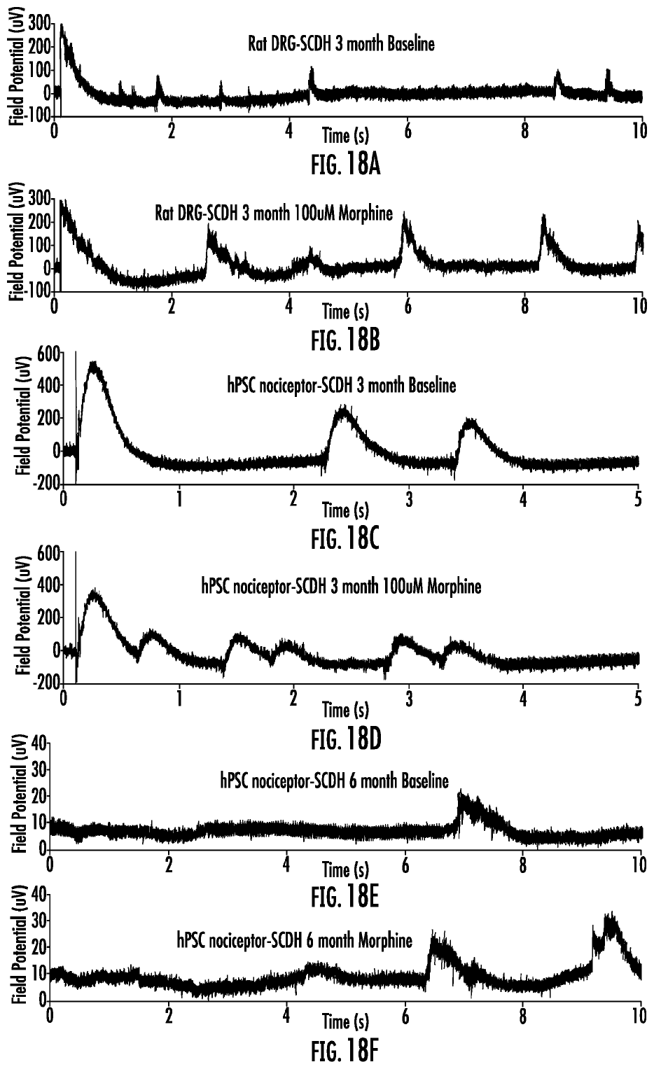

The pattern of bioelectric activity recorded from both embryonic rat DRG-SCDH and hPSC-derived nociceptor-dorsal spinal cord cocultures continues to evolve as cell types present and functional synaptic connections formed continue to mature. After 3 months in culture, semi-spontaneous burst firing appears in dorsal spinal cord spheroids of both embryonic rat DRG-SCDH and hPSC-derived nociceptor-SCDH cocultures (FIG. 18A,C). After application of 100 uM morphine, the semi-spontaneous burst firing increases in frequency and/or amplitude (FIG. 18B,D). After 6 months in culture, fully spontaneous burst firing is observed in the SCDH spheroid of hPSC-derived nociceptor-SCDH cocultures (FIG. 18E). Application of 100 uM morphine again increases the frequency and amplitude of these spontaneous bursts of activity. Thus, as in young cultures, morphine application in older cultures again increases the excitability of the SCDH spheroids when cocultured with peripheral nerve tissue. However, due to changes in the underlying physical synaptic connections within the more mature tissue, morphine-induced hyperexcitability manifests as increased frequency and amplitude of semi- and fully spontaneous burst firing rather than enhancing the duration of the initial, evoked response to afferent excitation.

The data presented here indicates that the physiology of the individual cells and synaptic connectivity within these cultures continue to mature for months in culture. The maturation of this physiology generates more complex and intricate macroscopic physiology than that of younger cultures. In younger cultures, only an immediate response to afferent stimulation is observed, generating a waveform with at least three meaningful components as described in Pollard et al. 2021. In older cultures, semi-spontaneous and fully spontaneous burst firing patterns characterized as large, repetitive, positive-going peaks emerge. Semi-spontaneous bursting occurs several seconds after stimulation. This time frame is too long to be considered directly evoked but cannot be considered spontaneous due to the initial stimulation necessary to generate them. Fully spontaneous bursting is recorded in complete absence of any stimulation. The frequency and amplitude of both semi-spontaneous and fully spontaneous burst firing events are sensitive to opioid receptor activation, confirming that they are informative for understanding the actions of analgesics in this model system.

Embryonic rat and human neurons in our culture systems likely develop spontaneous activity over time in culture due to changes in the array of transmembrane ion channels expressed in some or all of the neuronal subtypes as the neurons mature. After functional synapses have formed, there is always a small probability of spontaneous release of neurotransmitter at every synapse. This means that there is a constant trickle of a small amount excitatory neurotransmitter released and excitatory synapses. In young cultures, this small amount of spontaneous neurotransmitter release is likely not enough to result in excitation of the entire postsynaptic cell to the point of action potential firing due to low expression of neurotransmitter receptor. However, even the small amount of excitation resulting from spontaneous release of neurotransmitter can cause recruitment of increased amounts of neurotransmitter receptor to the post-synaptic cell membrane and increased expression of neurotransmitter receptor protein. This results in increased amounts of neurotransmitter receptor present at the post synaptic cell membrane as the neurons mature. Thus, the same amount of spontaneous neurotransmitter release will result in a larger post-synaptic depolarization, excitation of the post synaptic cell to the point of action potential firing, and subsequent downstream excitation of additional efferently connected neurons. This coordinated, connected network of neurons firing simultaneously will produce the type of spontaneous or semi-spontaneous burst firing patterns observed in the older rodent and human microphysiological cultures.

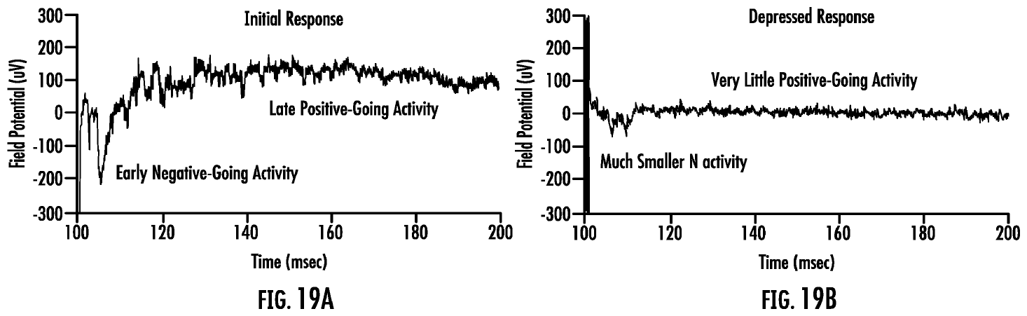

The important physiological information obtained from these constructs regarding the effects of morphine, and presumably other analgesics, is derived from the ability to measure the different waveform components before and after analgesic treatment. This is not possible if the stimulation protocol itself results in long-term depression of the trace because the information we need to obtain is lost. FIG. 19 describes an example of such depression. N1, N2, and P1 are clearly identifiable in the initial trace in panel A, but the rat stimulation protocol alone causes pronounced depression of the waveform. This makes it impossible to measure the same waveform components before and after treatment because the stimulation protocol itself causes their disappearance.

### Example 3

### Data Analysis

The microphysiological systems described in the present disclosure generate thousands of waveforms and more data than could possibly be analyzed by hand. The following algorithm was written in Igor Pro to aid in automated analysis of this many waveforms. All the waveforms associated with all samples from a given experiment are saved within a single Igor file. The algorithm then cycles through each trace within the file to identify positive-going burst firing events. The algorithm sequentially analyzes each trace in 100 msec segments. The algorithm first finds the maximum data value within the 100 msec window. If this maximum point is greater than three times the average noise of the recording and is a local maximum then the data point meets the criteria for being a burst firing event. If the conditions are met, the algorithm finds the amplitude and timepoint associated with the peak of this event and exports it to a data table for further analysis. Overall, a series hundreds or thousands of traces comprised of X and Y data points are input to the algorithm and the algorithm exports a series of X and Y points that are associated with the peak of each burst firing event in the trace. The average amplitude and distribution of inter-burst intervals of semi-spontaneous or fully spontaneous burst firing events under control and analgesic-treated conditions across the experiment can then be easily calculated from the resulting data table to quantitatively describe the effects of analgesics on culture excitability and compare them to the effects of morphine.

There are a number of potential descriptive metrics that may be important for understanding the physiology observed in these cultures by comparing them across control and analgesic-treated conditions. The code below demonstrates automated calculation of the amplitude of the spontaneous bursts, the latency to evoked and semi-spontaneous bursts, the inter-burst interval between semi-spontaneous and fully spontaneous bursts, and the duration of evoked, semi-spontaneous, and fully spontaneous burst firing. Future automated analysis code will be written to describe the duration of evoked, semi-spontaneous, and fully spontaneous bursts, and defining characteristics of the shape of the bursts including the initial slope of the curve, the time to the peak, and the decay time.

Below is the core of the peak finding algorithm. It finds the maximum value within a given window and, if it meets the specified conditions, it exports the X (time) values to one column of a data table, the Y (amplitude) values to another, and increases the count of spontaneous bursts by 1. This algorithm is written in Igor's native programming language. It is entirely custom and novel other than the built in Igor commands.

function pwaverecursedfr(t,x,y,l)

variable t
variable x
variable y
variable 1
Wave/Z w = WaveRefIndexedDFR(:, y)
wave PwaveYs
wave PwaveXs
wave wavecount
make/o /n=1 noise=((wavemax(w, 0,0.01)+wavemax(w, 0.01,0.02)+wavemax(w,
0.02,0.03)+wavemax(w, 0.03,0.04)+wavemax(w, 0.04,0.05)+wavemax(w,
0.05,0.06)+wavemax(w, 0.06,0.07)+wavemax(w, 0.07,0.08)+wavemax(w, 0.08,0.09))/9)
if (t == 0.15 && wavemax(w,t,t+0.15)>2*noise[0])
PwaveYs[x][y]-wavemax(w,t,t+0.15)
findlevel /Q /R=(t,t+0.15) w, wavemax(w, t,t+0.15)
PwaveXs[x][y]=V_LevelX
wavecount[y]=wavecount[y]+1
t=t+0.15
endif
if (t>0.15)
findlevel /Q /R=(t,t+0.1) w, wavemax(w, t,t+0.1)
make/o /n=1 xmax=V_levelX
endif
if (wavemax(w, t,t+0.1)>3*noise[0] && wavemax(w, t,t+0.1)>wavemax(w, xmax[0]−
0.05,xmax[0]−0.001) && wavemax(w, t,t+0.1)>wavemax(w, xmax[0]+0.001,xmax[0]+0.05) &&
(wavemax(w, xmax[0]−0.05,xmax[0]+0.05)−wavemin(w, xmax[0]−
0.05,xmax[0]+0.05))>4*noise[0])
PwaveYs[x][y]=wavemax(w, t,t+0.1)
findlevel /Q /R=(t,t+0.1) w, wavemax(w, t,t+0.1)
PwaveXs[x][y]=V_LevelX
wavecount[y]=wavecount[y]+1
endif
if (t<9.9)
pwaverecursedfr(t+0.1,x+1,y,1)
endif
if (t>9.9 && y<1*10)
pwaverecursedfr(0.1, 0, y+1, 1)
endif
end

Below is another function that repeats the above function but with different starting values to direct the algorithm to analyze all the traces in the file. As written, the code will analyze the first 140 traces in the file, but it can be extended indefinitely.

Function findpwavesindex 1( )
make/o /n=(100, 140) PwaveYs=0
make/o /n=(100,140) PwaveXs=0
make/o /n=140 wavecount=0
pwaverecursedfr(0.15,0,0,1)
pwaverecursedfr(0.15,0,11,2)
pwaverecursedfr(0.15,0,21,3)
pwaverecursedfr(0.15,0,31,4)
pwaverecursedfr(0.15,0,41,5)
pwaverecursedfr(0.15,0,51,6)
pwaverecursedfr(0.15,0,61,7)
pwaverecursedfr(0.15,0,71,8)
pwaverecursedfr(0.15,0,81,9)
pwaverecursedfr(0.15,0,91,10)
pwaverecursedfr(0.15,0,101,11)
pwaverecursedfr(0.15,0,111,12)
pwaverecursedfr(0.15,0,121,13)
pwaverecursedfr(0.15,0,131,14)
End

Finally, below is a series of functions that take the data exported by the peak finding algorithm and performs various calculations with it. It averages the number of peaks found across the 10 trials related to each treatment, it complies the exported amplitudes and latencies into a presentation format, it calculates the average amplitude and latency of the first 5 peaks in every trace, and it calculates all of the inter-peak intervals to generate the distribution of inter-peak intervals.

The function below averages the total number of burst firing events detected for each group of 10 traces. Treatments were performed in ten minute increments, with one recording per minute, so each group of 10 traces represents one treatment. For example, the first ten traces represent the baseline period, the second ten traces represent a sham treatment, the third ten traces represent another sham treatment, the forth ten traces represent morphine treatment, the fifth ten traces represent a second morphine treatment, etc. So this code is generating an average wave-count value that is representative of all the traces observed under each condition.

function compilecounts( )
wave wavecount
make/o /n=16 avgwavecnt-0
avgwavecnt[0]=mean(wavecount,0,9)
avgwavecnt[1]=mean(wavecount,10,19)
avgwavecnt[2]=mean(wavecount,20,29)
avgwavecnt[3]=mean(wavecount,30,39)
avgwavecnt[4]=mean(wavecount,40,49)
avgwavecnt[5]=mean(wavecount,50,59)
avgwavecnt[6]=mean(wavecount,60,69)
avgwavecnt[7]=mean(wavecount,70,79)
avgwavecnt[8]=mean(wavecount,80,89)
avgwavecnt[9]=mean(wavecount,90,99)
avgwavecnt[10]=mean(wavecount,100,109)
avgwavecnt[11]=mean(wavecount,110,119)
avgwavecnt[12]=mean(wavecount,120,129)
avgwavecnt[13]=mean(wavecount,130,139)
avgwavecnt[14]=mean(wavecount,140,149)
avgwavecnt[15]=mean(wavecount,150,159)
avgwavecnt[16]=mean(wavecount,160,169)
avgwavecnt[17]=mean(wavecount,170,179)
avgwavecnt[18]=mean(wavecount,180,189)
avgwavecnt[19]=mean(wavecount,190,199)
avgwavecnt[20]=mean(wavecount,200,209)
avgwavecnt[21]=mean(wavecount,210,219)
end

The function below creates data columns that will be populated later and then executes the functions that will populate the created data columns.

function compileAMPlat( )
make /o /n=(10,160) cleanPwaveYs
make /o /n=(10,160) cleanPwaveXs
recAMPlat(0,0,0,1)
recAMPlat(0,51,0,2)
recAMPlat(0,101,0,3)
recAMPlat(0,151,0,4)
end

The core peak picking function creates an X and Y column for each 150 msec interval in each trace and initially populates them all with zeroes. If it then detects a peak in that 150 msec interval it replaces the original zero value the X and Y column associated with that interval with the time and amplitude values, respectively. If it does not detect a peak, then it leaves the zero in place. This helps to create simplified images that represent entire experiments, but makes it very difficult to calculate simple averages. The function below copies the exported data from the peak-picking algorithm into the new data columns created above, and then deletes all of the zeroes leaving behind only the time and amplitude values related to successfully detected peaks and making it much simpler to calculate average data.

function recAMPlat(r,col,row,k)
variable r
variable row
variable col
variable k
wave PwaveYs
wave PwaveXs
wave cleanPwaveYs
wave cleanPwaveXs
if (PwaveXs[r][col]>0 && r<99)
cleanPwave Ys[row][col]=Pwave Ys[r][col]
cleanPwaveXs[row][col]=PwaveXs[r][col]
recAMPlat(r+1,col,row+1,k)
endif
if (PwaveXs[r][col]==0 && r<99)
recAMPlat(r+1,col,row,k)
endif
if (r==99 && col<k*50)
recAMPlat(0,col+1,0,k)
endif
end

The function below acts on the data columns with the zeroes removed that were just created above. It first creates new data columns to be populated with the average amplitude and latency values, and then populates those columns. This code is set up to compare the amplitude and latency of the first burst of activity across all treatments (or groups of 10 traces), then compare the amplitude and latency of the second burst, then the third, then the fourth, then the fifth. All of these latencies and amplitudes can then be exported to a stats program for analysis.

function extractAMPlat(k)
variable k
wave cleanpwaveYs
wave cleanpwaveXs
make /o /n=16 avgwave1amp
make /o /n=16 avgwave2amp
make /o /n=16 avgwave3amp
make /o /n=16 avgwave4amp
make /o /n=16 avgwave5amp
make /o /n=16 avgwave1lat
make /o /n=16 avgwave2lat
make /o /n=16 avgwave3lat
make /o /n=16 avgwave4lat
make /o /n=16 avgwave5lat
matrixtranspose cleanpwaveYs
matrixtranspose cleanpwaveXs
make/o /n=160 pwave1amp=cleanpwaveYs[p]
make/o /n=160 pwave2amp=cleanpwaveYs[p+k*10]
make/o /n=160 pwave3amp=cleanpwaveYs[p+k*10*2]
make/o /n=160 pwave4amp=cleanpwaveYs[p+k*10*3]
make/o /n=160 pwave5amp-cleanpwaveYs[p+k*10*4]
make/o /n=160 pwavecamp-cleanpwaveYs[p+k*10*5]
make/o /n=160 pwave7amp=cleanpwaveYs[p+k*10*6]
make/o /n=160 pwave8amp=cleanpwaveYs[p+k*10*7]
make/o /n=160 pwave9amp=cleanpwaveYs[p+k*10*8]
make/o /n=160 pwave10amp=cleanpwaveYs[p+k*10*9]
make/o /n=160 pwave1lat=cleanpwaveXs[p]
make/o /n=160 pwave2lat-cleanpwaveXs[p+k*10]
make/o /n=160 pwave3lat=cleanpwaveXs[p+k*10*2]
make/o /n=160 pwave4lat=cleanpwaveXs[p+k*10*3]
make/o /n=160 pwave5lat=cleanpwaveXs[p+k*10*4]
make/o /n=160 pwave6lat-cleanpwaveXs[p+k*10*5]
make/o /n=160 pwave7lat=cleanpwaveXs[p+k*10*6]
make/o /n=160 pwave8lat-cleanpwaveXs[p+k*10*7]
make/o /n=160 pwave9lat=cleanpwaveXs[p+k*10*8]
make/o /n=160 pwave10lat=cleanpwaveXs[p+k*10*9]
avgwave1amp[0]=mean(pwave1amp,0,9)
avgwave1amp[1]=mean(pwave1amp,10,19)
avgwave1amp[2]=mean(pwave1amp,20,29)
avgwave1amp[3]=mean(pwave1amp,30,39)
avgwave1amp[4]=mean(pwave1amp,40,49)
avgwave1amp[5]=mean(pwave1amp,50,59)
avgwave1amp[6]=mean(pwave1amp,60,69)
avgwave1amp[7]=mean(pwave1amp,70,79)
avgwave1amp[8]=mean(pwave1amp,80,89)
avgwave1amp[9]=mean(pwave1amp,90,99)
avgwave1amp[10]=mean(pwave1amp,100,109)
avgwave1amp[11]=mean(pwave1amp,110,119)
avgwave1amp[12]=mean(pwave1amp,120,129)
avgwave1amp[13]=mean(pwave1amp,130,139)
avgwave1amp[14]=mean(pwave1amp,140,149)
avgwave1amp[15]=mean(pwave1amp,150,159)
avgwave1amp[16]=mean(pwave1amp,160,169)
avgwave1amp[17]=mean(pwave1amp,170,179)
avgwave1amp[18]=mean(pwave1amp,180,189)
avgwave1amp[19]=mean(pwave1amp,190,199)
avgwave1amp[20]=mean(pwave1amp,200,209)
avgwave1amp[21]=mean(pwave1amp,210,219)
avgwave1lat[0]=mean(pwave1lat,0,9)
avgwave1lat[1]=mean(pwave1lat,10,19)
avgwave1lat[2]=mean(pwave1lat,20,29)
avgwave1lat[3]=mean(pwave1lat,30,39)
avgwave1lat[4]=mean(pwave1lat,40,49)
avgwave1lat[5]=mean(pwave1lat,50,59)
avgwave1lat[6]=mean(pwave1lat,60,69)
avgwave1lat[7]=mean(pwave1lat,70,79)
avgwave1lat[8]=mean(pwave1lat,80,89)
avgwave1lat[9]=mean(pwave1lat,90,99)
avgwave1lat[10]=mean(pwave1lat,100,109)
avgwave1lat[11]=mean(pwave1lat, 110,119)
avgwave1lat[12]=mean(pwave1lat,120,129)
avgwave1lat[13]=mean(pwave1lat,130,139)
avgwave1lat[14]=mean(pwave1lat, 140,149)
avgwave1lat[15]=mean(pwave1lat,150,159)
avgwave1lat[16]=mean(pwave1lat,160,169)
avgwave1lat[17]=mean(pwave1lat,170,179)
avgwave1lat[18]=mean(pwave1lat,180,189)
avgwave1lat[19]=mean(pwave1lat, 190,199)
avgwave1lat[20]=mean(pwave1lat,200,209)
avgwave1lat[21]=mean(pwave1lat,210,219)
avgwave2amp[0]=mean(pwave2amp,0,9)
avgwave2amp[1]=mean(pwave2amp,10,19)
avgwave2amp[2]=mean(pwave2amp,20,29)
avgwave2amp[3]=mean(pwave2amp,30,39)
avgwave2amp[4]=mean(pwave2amp,40,49)
avgwave2amp[5]=mean(pwave2amp,50,59)
avgwave2amp[6]=mean(pwave2amp,60,69)
avgwave2amp[7]=mean(pwave2amp,70,79)
avgwave2amp[8]=mean(pwave2amp,80,89)
avgwave2amp[9]=mean(pwave2amp,90,99)
avgwave2amp[10]=mean(pwave2amp,100,109)
avgwave2amp[11]=mean(pwave2amp,110,119)
avgwave2amp[12]=mean(pwave2amp,120,129)
avgwave2amp[13]=mean(pwave2amp,130,139)
avgwave2amp[14]=mean(pwave2amp,140,149)
avgwave2amp[15]=mean(pwave2amp,150,159)
avgwave2amp[16]=mean(pwave2amp,160,169)
avgwave2amp[17]=mean(pwave2amp,170,179)
avgwave2amp[18]=mean(pwave2amp,180,189)
avgwave2amp[19]=mean(pwave2amp,190,199)
avgwave2amp[20]=mean(pwave2amp,200,209)
avgwave2amp[21]=mean(pwave2amp,210,219)
avgwave2lat[0]=mean(pwave2lat,0,9)
avgwave2lat[1]=mean(pwave2lat,10,19)
avgwave2lat[2]=mean(pwave2lat,20,29)
avgwave2lat[3]=mean(pwave2lat,30,39)
avgwave2lat[4]=mean(pwave2lat,40,49)
avgwave2lat[5]=mean(pwave2lat,50,59)
avgwave2lat[6]=mean(pwave2lat,60,69)
avgwave2lat[7]=mean(pwave2lat,70,79)
avgwave2lat[8]=mean(pwave2lat,80,89)
avgwave2lat[9]=mean(pwave2lat,90,99)
avgwave2lat[10]=mean(pwave2lat,100,109)
avgwave2lat[11]=mean(pwave2lat,110,119)
avgwave2lat[12]=mean(pwave2lat,120,129)
avgwave2lat[13]=mean(pwave2lat,130,139)
avgwave2lat[14]=mean(pwave2lat,140,149)
avgwave2lat[15]=mean(pwave2lat,150,159)
avgwave2lat[16]=mean(pwave2lat,160,169)
avgwave2lat[17]=mean(pwave2lat,170,179)
avgwave2lat[18]=mean(pwave2lat,180,189)
avgwave2lat[19]=mean(pwave2lat,190,199)
avgwave2lat[20]=mean(pwave2lat,200,209)
avgwave2lat[21]=mean(pwave2lat,210,219)
avgwave3amp[0]=mean(pwave3amp,0,9)
avgwave3amp[1]=mean(pwave3amp,10,19)
avgwave3amp[2]=mean(pwave3amp,20,29)
avgwave3amp[3]=mean(pwave3amp,30,39)
avgwave3amp[4]=mean(pwave3amp,40,49)
avgwave3amp[5]=mean(pwave3amp,50,59)
avgwave3amp[6]=mean(pwave3amp,60,69)
avgwave3amp[7]=mean(pwave3amp,70,79)
avgwave3amp[8]=mean(pwave3amp,80,89)
avgwave3amp[9]=mean(pwave3amp,90,99)
avgwave3amp[10]=mean(pwave3amp,100,109)
avgwave3amp[11]=mean(pwave3amp,110,119)
avgwave3amp[12]=mean(pwave3amp,120,129)
avgwave3amp[13]=mean(pwave3amp,130,139)
avgwave3amp[14]=mean(pwave3amp,140,149)
avgwave3amp[15]=mean(pwave3amp,150,159)
avgwave3amp[16]=mean(pwave3amp,160,169)
avgwave3amp[17]=mean(pwave3amp,170,179)
avgwave3amp[18]=mean(pwave3amp,180,189)
avgwave3amp[19]=mean(pwave3amp,190,199)
avgwave3amp[20]=mean(pwave3amp,200,209)
avgwave3amp[21]=mean(pwave3amp,210,219)
avgwave3lat[0]=mean(pwave3lat,0,9)
avgwave3lat[1]=mean(pwave3lat,10,19)
avgwave3lat[2]=mean(pwave3lat,20,29)
avgwave3lat[3]=mean(pwave3lat,30,39)
avgwave3lat[4]=mean(pwave3lat,40,49)
avgwave3lat[5]=mean(pwave3lat,50,59)
avgwave3lat[6]=mean(pwave3lat,60,69)
avgwave3lat[7]=mean(pwave3lat,70,79)
avgwave3lat[8]=mean(pwave3lat,80,89)
avgwave3lat[9]=mean(pwave3lat,90,99)
avgwave3lat[10]=mean(pwave3lat,100,109)
avgwave3lat[11]=mean(pwave3lat,110,119)
avgwave3lat[12]=mean(pwave3lat,120,129)
avgwave3lat[13]=mean(pwave3lat,130,139)
avgwave3lat[14]=mean(pwave3lat,140,149)
avgwave3lat[15]=mean(pwave3lat,150,159)
avgwave3lat[16]=mean(pwave3lat,160,169)
avgwave3lat[17]=mean(pwave3lat,170,179)
avgwave3lat[18]=mean(pwave3lat,180,189)
avgwave3lat[19]=mean(pwave3lat,190,199)
avgwave3lat[20]=mean(pwave3lat,200,209)
avgwave3lat[21]=mean(pwave3lat,210,219)
avgwave4amp[0]=mean(pwave4amp,0,9)
avgwave4amp[1]=mean(pwave4amp,10,19)
avgwave4amp[2]=mean(pwave4amp,20,29)
avgwave4amp[3]=mean(pwave4amp,30,39)
avgwave4amp[4]=mean(pwave4amp,40,49)
avgwave4amp[5]=mean(pwave4amp,50,59)
avgwave4amp[6]=mean(pwave4amp,60,69)
avgwave4amp[7]=mean(pwave4amp,70,79)
avgwave4amp[8]=mean(pwave4amp,80,89)
avgwave4amp[9]=mean(pwave4amp,90,99)
avgwave4amp[10]=mean(pwave4amp,100,109)
avgwave4amp[11]=mean(pwave4amp,110,119)
avgwave4amp[12]=mean(pwave4amp,120,129)
avgwave4amp[13]=mean(pwave4amp,130,139)
avgwave4amp[14]=mean(pwave4amp,140,149)
avgwave4amp[15]=mean(pwave4amp,150,159)
avgwave4amp[16]=mean(pwave4amp,160,169)
avgwave4amp[17]=mean(pwave4amp,170,179)
avgwave4amp[18]=mean(pwave4amp,180,189)
avgwave4amp[19]=mean(pwave4amp,190,199)
avgwave4amp[20]=mean(pwave4amp,200,209)
avgwave4amp[21]=mean(pwave4amp,210,219)
avgwave4lat[0]=mean(pwave4lat,0,9)
avgwave4lat[1]=mean(pwave4lat,10,19)
avgwave4lat[2]=mean(pwave4lat,20,29)
avgwave4lat[3]=mean(pwave4lat,30,39)
avgwave4lat[4]=mean(pwave4lat,40,49)
avgwave4lat[5]=mean(pwave4lat,50,59)
avgwave4lat[6]=mean(pwave4lat,60,69)
avgwave4lat[7]=mean(pwave4lat,70,79)
avgwave4lat[8]=mean(pwave4lat,80,89)
avgwave4lat[9]=mean(pwave4lat,90,99)
avgwave4lat[10]=mean(pwave4lat,100,109)
avgwave4lat[11]=mean(pwave4lat,110,119)
avgwave4lat[12]=mean(pwave4lat,120,129)
avgwave4lat[13]=mean(pwave4lat,130,139)
avgwave4lat[14]=mean(pwave4lat,140,149)
avgwave4lat[15]=mean(pwave4lat,150,159)
avgwave4lat[16]=mean(pwave4lat,160,169)
avgwave4lat[17]=mean(pwave4lat,170,179)
avgwave4lat[18]=mean(pwave4lat,180,189)
avgwave4lat[19]=mean(pwave4lat,190,199)
avgwave4lat[20]=mean(pwave4lat,200,209)
avgwave4lat[21]=mean(pwave4lat,210,219)
avgwave5amp[0]=mean(pwave5amp,0,9)
avgwave5amp[1]=mean(pwave5amp,10,19)
avgwave5amp[2]=mean(pwave5amp,20,29)
avgwave5amp[3]=mean(pwave5amp,30,39)
avgwave5amp[4]=mean(pwave5amp,40,49)
avgwave5amp[5]=mean(pwave5amp,50,59)
avgwave5amp[6]=mean(pwave5amp,60,69)
avgwave5amp[7]=mean(pwave5amp,70,79)
avgwave5amp[8]=mean(pwave5amp,80,89)
avgwave5amp[9]=mean(pwave5amp,90,99)
avgwave5amp[10]=mean(pwave5amp,100,109)
avgwave5amp[11]=mean(pwave5amp,110,119)
avgwave5amp[12]=mean(pwave5amp,120,129)
avgwave5amp[13]=mean(pwave5amp,130,139)
avgwave5amp[14]=mean(pwave5amp,140,149)
avgwave5amp[15]=mean(pwave5amp,150,159)
avgwave5amp[16]=mean(pwave5amp,160,169)
avgwave5amp[17]=mean(pwave5amp,170,179)
avgwave5amp[18]=mean(pwave5amp,180,189)
avgwave5amp[19]=mean(pwave5amp,190,199)
avgwave5amp[20]=mean(pwave5amp,200,209)
avgwave5amp[21]=mean(pwave5amp,210,219)
avgwave5lat[0]=mean(pwave5lat,0,9)
avgwave5lat[1]=mean(pwave5lat,10,19)
avgwave5lat[2]=mean(pwave5lat,20,29)
avgwave5lat[3]=mean(pwave5lat,30,39)
avgwave5lat[4]=mean(pwave5lat,40,49)
avgwave5lat[5]=mean(pwave5lat,50,59)
avgwave5lat[6]=mean(pwave5lat,60,69)
avgwave5lat[7]=mean(pwave5lat,70,79)
avgwave5lat[8]=mean(pwave5lat,80,89)
avgwave5lat[9]=mean(pwave5lat,90,99)
avgwave5lat[10]=mean(pwave5lat,100,109)
avgwave5lat[11]=mean(pwave5lat,110,119)
avgwave5lat[12]=mean(pwave5lat,120,129)
avgwave5lat[13]=mean(pwave5lat,130,139)
avgwave5lat[14]=mean(pwave5lat,140, 149)
avgwave5lat[15]=mean(pwave5lat,150,159)
avgwave5lat[16]=mean(pwave5lat,160,169)
avgwave5lat[17]=mean(pwave5lat,170,179)
avgwave5lat[18]=mean(pwave5lat,180,189)
avgwave5lat[19]=mean(pwave5lat,190, 199)
avgwave5lat[20]=mean(pwave5lat,200,209)
avgwave5lat[21]=mean(pwave5lat,210,219)
end

This function creates a new data table to be populated by the next function, and then executes the two functions to populate it.

Function peakINTERVALS( )

wave pwaveXs
make/o /n=(100,140) trimPX=0
delete0px(0,0,0,0)
delete0px70(0,70,0,70)
doipi( )
end

This function again copies the original output of the latencies from the initial peak picking algorithm to the newly created data columns and deletes any zeroes from it leaving just a list of the time points associated with successfully detected peaks.

Function delete0px(a,b,r,c)
variable a
variable b
variable r
variable c
wave trimpx
wave pwaveXs
if (pwaveXs[r][c]==0 && r<100 && c<70)
delete0px(a,b,r+1,c)
endif
if (pwaveXs[r][c]>0 && r<100 && c<70)
trimPX[a][b]=pwaveXs[r][c]
delete0px(a+1,b,r+1,c)
endif
if (r==100 && c<70)
delete0px(0,b+1,0,c+1)
endif
end

This function completes the deletion of zeros.

Function delete0px70(a,b,r,c)
variable a
variable b
variable r
variable c
wave trimpx
wave pwaveXs
if (pwaveXs[r][c]==0 && r<100 && c<140)
delete0px70(a,b,r+1,c)
endif
if (pwaveXs[r][c]>0 && r<100 && c<140)
trimPX[a][b]=pwaveXs[r][c]
delete0px70(a+1,b,r+1,c)
endif
if (r==100 && c<140)
delete0px70(0,b+1,0,c+1)
endif
end

This function creates a new data column to be populated by the next function, and then executes the function to populate it.

function doIPI( )
make/o /n=(100,140) IPIwave-0
IPI(0,0)
end

The inter-burst interval is the time between two bursts. This means that two (or more) bursts need to be detected within the same trace in order to start calculating inter-burst intervals. This function first confirms that there are at least two peaks detected, then finds the difference in time between the two peaks, and then stores that difference in a new data table. Then, it repeats sequentially through the remaining pairs of data points until one is a zero (indicating there are no more bursts to consider). Any leftover cells in the table are left as zeroes.

function IPI(a,b)
variable a,b
wave trimpx
wave IPIwave
if (trimpx[a][b]>0 && trimpx[a+1][b]>0 && b<140)
IPIwave[a][b]=trimpx[a+1][b]-trimpx[a][b]
IPI(a+1,b)
else
IPI(0,b+1)
endif
end

Again each series of 10 consecutive traces represent a single treatment group. This function splits the large data table created above into a single column for each treatment group, containing all inter-burst intervals calculated across the ten traces representative of that group. Including any cells that still have values of zero.

function splitITI( )
wave IPIwave
make/o /n=(10*100) wave0ITI=IPIwave[p]
make/o /n=(10*100) wave1ITI=IPIwave[p+(100*10)]
make/o /n=(10*100) wave2ITI=IPIwave[p+(2*100*10)]
make/o /n=(10*100) wave3ITI=IPIwave[p+(3*100*10)]
make/o /n=(10*100) wave4ITI-IPIwave[p+(4*100*10)]
make/o /n=(10*100) wave5ITI-IPIwave[p+(5*100*10)]
make/o /n=(10*100) wave6ITI=IPIwave[p+(6*100*10)]
make/o /n=(10*100) wave7ITI=IPIwave[p+(7*100*10)]
make/o /n=(10*100) wave8ITI=IPIwave[p+(8*100*10)]
make/o /n=(10*100) wave9ITI-IPIwave[p+(9*100*10)]
make/o /n=(10*100) wave10ITI=IPIwave[p+(10*100*10)]
make/o /n=(10*100) wave11ITI=IPIwave[p+(11*100*10)]
make/o /n=(10*100) wave12ITI=IPIwave[p+(12*100*10)]
make/o /n=(10*100) wave13ITI=IPIwave[p+(13*100*10)]
make/o /n=(10*100) wave14ITI-IPIwave[p+(14*100*10)]
end

This function executes all of the functions below to delete all of the extra zeroes from each of the now independent data columns. At the end there will be a single data column containing every inter-burst interval related to each treatment group in the experiment. These can be exported to a stats program for statistical analysis.

function delete0ITIs( )
delete0wave0ITI(0)
delete0wave1ITI(0)
delete0wave2ITI(0)
delete0wave3ITI(0)
delete0wave4ITI(0)
delete0wave5ITI(0)
delete0wave6ITI(0)
delete0wave7ITI(0)
delete0wave8ITI(0)
delete0wave9ITI(0)
delete0wave10ITI(0)
delete0wave11ITI(0)
delete0wave12ITI(0)
delete0wave13ITI(0)
delete0wave14ITI(0)
end
function delete0wave0ITI(a)
variable a
wave wave0ITI
if(wave0ITI[a]==0)
deletepoints a, 1,wave0ITI
delete0wave0ITI(a)
else
delete0wave0ITI(a+1)
endif
end
function delete0wave1ITI(a)
variable a
wave wave1ITI
if(wave1ITI[a]==0)
deletepoints a, 1, wave 1ITI
delete0wave1ITI(a)
else
delete0wave1ITI(a+1)
endif
end
function delete0wave2ITI(a)
variable a
wave wave2ITI
if(wave2ITI[a]==0)
deletepoints a, 1, wave2ITI
delete0wave2ITI(a)
else
delete0wave2ITI(a+1)
endif
end
function delete0wave3ITI(a)
variable a
wave wave3ITI
if(wave3ITI[a]==0)
deletepoints a, 1, wave3ITI
delete0wave3ITI(a)
else
delete0wave3ITI(a+1)
endif
end
function delete0wave4ITI(a)
variable a
wave wave4ITI
if(wave4ITI[a]==0)
deletepoints a, 1, wave4ITI
delete0wave4ITI(a)
else
delete0wave4ITI(a+1)
endif
end
function delete0wave5ITI(a)
variable a
wave wave5ITI
if(wave5ITI[a]==0)
deletepoints a, 1, wave5ITI
delete0wave5ITI(a)
else
delete0wave5ITI(a+1)
endif
end
function delete0wave6ITI(a)
variable a
wave wave6ITI
if(wave6ITI[a]==0)
deletepoints a, 1, wave6ITI
delete0wave6ITI(a)
else
delete0wave6ITI(a+1)
endif
end
function delete0wave7ITI(a)
variable a
wave wave7ITI
if(wave7ITI[a]==0)
deletepoints a, 1, wave7ITI
delete0wave7ITI(a)
else
delete0wave7ITI(a+1)
endif
end
function delete0wave8ITI(a)
variable a
wave wave8ITI
if(wave8ITI[a]==0)
deletepoints a, 1, wave8ITI
delete0wave8ITI(a)
else
delete0wave8ITI(a+1)
endif
end
function delete0wave9ITI(a)
variable a
wave wave9ITI
if(wave9ITI[a]==0)
deletepoints a, 1, wave9ITI
delete0wave9ITI(a)
else
delete0wave9ITI(a+1)
endif
end
function delete0wave10ITI(a)
variable a
wave wave10ITI
if(wave10ITI[a]==0)
deletepoints a, 1, wave10ITI
delete0wave10ITI(a)
else
delete0wave10ITI(a+1)
endif
end
function delete0wave11ITI(a)
variable a
wave wave11ITI
if(wave11ITI[a]==0)
deletepoints a, 1, wave11ITI
delete0wave11ITI(a)
else
delete0wave11ITI(a+1)
endif
end
function delete0wave12ITI(a)
variable a
wave wave12ITI
if(wave12ITI[a]==0)
deletepoints a, 1, wave12ITI
delete0wave12ITI(a)
else
delete0wave12ITI(a+1)
endif
end
function delete0wave13ITI(a)
variable a
wave wave13ITI
if(wave13ITI[a]==0)
deletepoints a, 1, wave13ITI
delete0wave13ITI(a)
else
delete0wave13ITI(a+1)
endif
end
function delete0wave14ITI(a)
variable a
wave wave14ITI
if(wave14ITI[a]==0)
deletepoints a, 1, wave 14ITI
delete0wave14ITI(a)
else
delete0wave14ITI(a+1)
endif
end

### REFERENCES

1. I. Bergh, G. Steen, M. Waern, B. Johansson, A. Oden, B. Sjöström, B. Steen, Pain and its relation to cognitive function and depressive symptoms: A Swedish population study of 70-year-old men and women. Journal of Pain and Symptom Management 26, 903-912 (2003).
2. L. Simon, Relieving Pain in America: A Blueprint for Transforming Prevention, Care, Education, and Research. Journal of Pain and Palliative Care Pharmacotherapy 26, 197-198 (2012).
3. A. I. Basbaum, D. M. Bautista, G. Scherrer, D. Julius, Cellular and molecular mechanisms of pain. Cell 139, 267-284 (2009).
4. J. Pergolizzi, K. Ahlbeck, D. Aldington, E. Alon, F. Coluzzi, A. Dahan, F. Huygen, M. Kocot-Kepska, A. C. Mangas, P. Mavrocordatos, B. Morlion, G. Muller-Schwefe, Nicolaou, C. Perez Hernandez, P. Sichere, M. Schafer, G. Varrassi, The development of chronic pain: physiological CHANGE necessitates a multidisciplinary approach to treatment. Curr Med Res Opin 29, 1127-1135 (2013).
5. P. Schmitt, Rehabilitation of Chronic Pain: A Multidisciplinary Approach. Journal of Rehabilitation 51, 72-75 (1985).
6. J. C. Ballantyne, N. S. Shain, Efficacy of Opioids for Chronic Pain: A Review of the Evidence. The Clinical Journal of Pain 24, 469-478 (2008).
7. N. Wilson, M. Kariisa, P. Seth, H. Smith IV, N. L. Davis Drug and Opioid-Involved Overdose Deaths—United States, 2017-2018. Morbidity and Mortality Weekly Report 69 (11), 290-297 (2020).
8. N. D. Volkow, F. S. Collins, The Role of Science in Addressing the Opioid Crisis. The New England Journal of Medicine 337, 391-394 (2017).
9. I. Kola, J. Landis, Can the pharmaceutical industry reduce attrition rates? Nature Reviews Drug Discovery 3, 711-715 (2004).
10. A. Skardal, T. Shupe, A. Atala, Organoid-on-a-chip and body-on-a-chip systems for drug screening and disease modeling. Drug Discov Today 21, 1399-1411(2016).
11. B. Zhang, M. Radisic, Organ-on-a-chip devices advance to market. Lab Chip 17, 2395-2420 (2017).
12. P. Goldstein, J. Gu, “Models for Studying Pain and Searching for Pain Killers in Vitro Electrophysiological Studies of Pain” in Molecular Pain, ZhuoM, Ed. (Springer, New York, NY, 2007), pp. 441-458.
13. A. Sertkaya, H. H. Wong, A. Jessup, T. Beleche, Key cost drivers of pharmaceutical clinical trials in the United States. Clin Trials 13, 117-126 (2016).
14. T. Hartung, Thoughts on limitations of animal models. Parkinsonism Relat Disord 14 Suppl 2, S81-83 (2008).
15. S. Boyce, R. Hill, “Discrepant Results from Preclinical and Clinical Studies on the Potential of Substance P-Receptor Antagonist Compounds as Analgesics” in Progress in Pain Research and Management, M. Devor, M. Rowbotham, Z. Wiesenfeld-Hallin, Eds. (IASP Press, Vienna, 2000), pp. 313-324.
16. M. S. Wallace, M. Rowbotham, G. J. Bennett, T. S. Jensen, R. Pladna, S. Quessy, A multicenter, double-blind, randomized, placebo-controlled crossover evaluation of a short course of 4030W92 in patients with chronic neuropathic pain Journal of Pain 3 (3), 227-233 (2002).
17. M. S. Wallace, M. Rowbotham, N. P. Katz, R. H. Dworkin, R. M. Dotson, S. Galer, R. L. Rauck, M. M. Backonja, S. N. Quessy, P. D. Meisner, A randomized, double-blind, placebo-controlled trial of a glycine antagonist in neuropathic pain. Neurology 59, 1694-1700 (2002).
18. K. Duval, H. Grover, L. H. Han, Y. Mou, A. F. Pegoraro, J. Fredberg, Z. Chen, Modeling Physiological Events in 2D vs. 3D Cell Culture. Physiology (Bethesda) 32,266-277 (2017).
19. S. Ahadian, R. Civitarese, D. Bannerman, M. H. Mohammadi, R. Lu, E. Wang, L. Davenport-Huyer, B. Lai, B. Zhang, Y. Zhao, S. Mandla, A. Korolj, M. Radisic, Organ-On-A-Chip Platforms: A Convergence of Advanced Materials, Cells, and Microscale Technologies. Adv Healthc Mater 7, (2018).
20. J. P. Wikswo, The relevance and potential roles of microphysiological systems in biology and medicine. Exp Biol Med (Maywood) 239, 1061-1072(2014).
21. R. D. Kamm, R. Bashir, N. Arora, R. D. Dar, M. U. Gillette, L. G. Griffith, M. L. Kemp, K. Kinlaw, M. Levin, A. C. Martin, T. C. McDevitt, R. M. Nerem, M. J. Powers, T. A. Saif, J. Sharpe, S. Takayama, S. Takeuchi, R. Weiss, K. Ye, H. G. Yevick, M. H. Zaman, Perspective: The promise of multi-cellular engineered living systems. APL Bioeng 2, 040901 (2018).
22. K. Yang, E. Kumamoto, H. Furue, M. Yoshimura, Capsaicin facilitates excitatory but not inhibitory synaptic transmission in substantia gelatinosa of the rat spinal cord. Neuroscience Letters 255, 135-138 (1998).
23. E. E. Benarroch, Dorsal horn circuitry complexity and implications for mechanisms of neuropathic pain. Neurology 86, 1060-1069 (2016).
24. A. J. Todd, Neuronal circuitry for pain processing in the dorsal horn. Nat Rev Neurosci 11, 823-836 (2010).
25. M. J. Millan, Descending Control of Pain. Progress in Neurobiology 66, 355-474 (2002).
26. A. Merighi, The histology, physiology, neurochemistry and circuitry of the substantia gelatinosa Rolandi (lamina II) in mammalian spinal cord. Prog Neurobiol 169, 91-134 (2018).
27. A. W. Gorlin, D. M. Rosenfeld, H. Ramakrishna, Intravenous sub-anesthetic ketamine for perioperative analgesia. J Anaesthesiol Clin Pharmacol 32, 160-167 (2016).
28. T. Kemp, C. Spike, C. Watt, A. J. Todd, The mu-opioid receptor (MOR1) is mainly restricted to neurons that do not contain GABA or glycine in the superficial dorsal horn of the rat spinal cord. Neuroscience 75, 1231-1238 (1996).
29. J. K. Wang, Pain relief by intrathecally applied morphine in man. Anesthesiology 50, 149-151 (1979).
30. B. Song, C. G. Marvizon, Dorsal horn neurons firing at high frequency, but not primary afferents, release opioid peptides that produce micro-opioid receptor internalization in the rat spinal cord. The Journal of Neuroscience 23, 9171-9184 (2003).
31. B. K. Lau, C. W. Vaughan, Descending modulation of pain: the GABA disinhibition hypothesis of analgesia. Curr Opin Neurobiol 29, 159-164 (2014).
32. A. Francois, S. A. Low, E. I. Sypek, A. J. Christensen, C. Sotoudeh, K. T. Beier, C. Ramakrishnan, K. D. Ritola, R. Sharif-Naeini, K. Deisseroth, S. L. Delp, R. C. Malenka, L. Luo, A. W. Hantman, G. Scherrer, A Brainstem-Spinal Cord Inhibitory Circuit for Mechanical Pain Modulation by GABA and Enkephalins. Neuron 93, 822-839 e826 (2017).
33. S. W. Johnson, Opioids excite dopamine neurons by hyperpolarization of local interneurons. The Journal of Neuroscience 12, 483-488 (1992).
34. A. Chen, Z. Guo, L. Fang, S. Bian, Application of Fused Organoid Models to Study Human Brain Development and Neural Disorders. Front Cell Neurosci 14, 133 (2020).
35. R. M. Marton, S. P. Pasca, Organoid and Assembloid Technologies for Investigating Cellular Crosstalk in Human Brain Development and Disease. Trends Cell Biol 30, 133-143 (2020).
36. J. L. Curley, M. J. Moore, Facile micropatterning of dual hydrogel systems for 3D models of neurite outgrowth. J Biomed Mater Res A 99, 532-543 (2011).
37. R. M. Huval, O. H. Miller, J. L. Curley, Y. Fan, B. J. Hall, M. J. Moore, Microengineered peripheral nerve-on-a-chip for preclinical physiological testing. Lab Chip 15, 2221-2232 (2015).
38. P. Khoshakhlagh, A. Sivakumar, L. A. Pace, D. W. Sazer, M. J. Moore, Methods for fabrication and evaluation of a 3D microengineered model of myelinated peripheral nerve. J Neural Eng 15, 064001 (2018).
39. L. Kramer, H. T. Nguyen, E. Jacobs, L. McCoy, J. L. Curley, A. D. Sharma, M. J. Moore, Modeling chemotherapy-induced peripheral neuropathy using a Nerve-on-a-chip microphysiological system. ALTEX, (2020).
40. K. J. Pollard, B. Bolon, M. J. Moore, Comparative Analysis of Chemotherapy-Induced Peripheral Neuropathy in Bioengineered Sensory Nerve Tissue Distinguishes Mechanistic Differences in Early-Stage Vincristine-, Cisplatin-, and Paclitaxel-Induced Nerve Damage. Toxicological Sciences, (2021).
41. A. P. Balgude, X. Yu, A. Szymanski, R. V. Bellamkonda, Agarose gel stiffness determines rate of DRG neurite extension in 3D cultures. Biomaterials 22 (10), (2001).
42. L. A. Flanagan, T.-E. Ju, B. Marg, M. Osterfeld, P. A. Janmey, Neurite branching on deformable surfaces. Developmental Neuroscience 13 (18), 2411-2415 (2002).
43. J. B. Leach, X. Q. Brown, J. G. Jacot, P. A. Dimilla, J. Y. Wong, Neurite outgrowth and branching of PC12 cells on very soft substrates sharply decreases below a threshold of substrate rigidity. J Neural Eng 4, 26-34 (2007).
44. J. Lantoine, T. Grevesse, A. Villers, G. Delhaye, C. Mestdagh, M. Versaevel, D. Mohammed, C. Bruyere, L. Alaimo, S. P. Lacour, L. Ris, S. Gabriele, Matrix stiffness modulates formation and activity of neuronal networks of controlled architectures. Biomaterials 89, 14-24 (2016).
45. P. Khoshakhlagh, M. J. Moore, Photoreactive interpenetrating network of hyaluronic acid and Puramatrix as a selectively tunable scaffold for neurite growth. Acta Biomater 16, 23-34 (2015).
46. P. Khoshakhlagh, D. A. Bowser, J. Q. Brown, M. J. Moore, Comparison of visible and UVA phototoxicity in neural culture systems micropatterned with digital projection photolithography. J Biomed Mater Res A 107, 134-144 (2019).
47. E. S. Boyden, F. Zhang, E. Bamberg, G. Nagel, K. Deisseroth, Millisecond-timescale, genetically targeted optical control of neural activity. Nat Neurosci 8, 1263-1268 (2005).
48. B. Moeyaert, G. Holt, R. Madangopal, A. Perez-Alvarez, B. C. Fearey, N. F. Trojanowski, J. Ledderose, T. A. Zolnik, A. Das, D. Patel, T. A. Brown, R. N. S. Sachdev, B. J. Eickholt, M. E. Larkum, G. G. Turrigiano, H. Dana, C. E. Gee, T. G. Oertner, B. T. Hope, E. R. Schreiter, Improved methods for marking active neuron populations. Nat Commun 9, 4440 (2018).
49. X.-Y. Hua, A. Saria, R. Gamse, E. Theodorsson-Norheim, E. Brodin, J. M. Lundbert, Capsaicin induced release of multiple tachykinins (substance P, neurokinin A and eledoisin-like material) from guinea-pig spinal cord and ureter. Neuroscience 19, 313-319 (1986).
50. A. Merighi, R. Bardoni, C. Salio, L. Lossi, F. Ferrini, M. Prandini, M. Zonta, S. Gustincich, G. Carmignoto, Presynaptic functional trkB receptors mediate the release of excitatory neurotransmitters from primary afferent terminals in lamina II (substantia gelatinosa) of postnatal rat spinal cord. Dev Neurobiol 68, 457-475 (2008).
51. N. S. Simons-Weidenmaier, M. Weber, C. F. Plappert, P. K. Pilz, S. Schmid, Synaptic depression and short-term habituation are located in the sensory part of the mammalian startle pathway. BMC Neurosci 7, 38 (2006).
52. H. Baba, M. Yoshimura, S. Nishi, K. Shimoji, Synaptic responses of substantia gelatinosa neurones to dorsal column stimulation in rat spinal cord in vitro. Journal of Physiology 478, 87-99(1994).
53. A. E. Dubin, A. Patapoutian, Nociceptors: the sensors of the pain pathway. J Clin Invest 120, 3760-3772 (2010).
54. M. J. Millan, The induction of pain: an integrative review. Progress in Neurobiology 57, 1-164 (1999).
55. K. J. Pollard, A. D. Sharma, M. J. Moore, Neural microphysiological systems for in vitro modeling of peripheral nervous system disorders. Bioelectronics in Medicine 2, (2019).
56. G. Sun, W. Liu, Z. Fan, D. Zhang, Y. Han, L. Xu, J. Qi, S. Zhang, B. T. Gao, X. Bai, J. Li, R. Chai, H. Wang, The Three-Dimensional Culture System with Matrigel and Neurotrophic Factors Preserves the Structure and Function of Spiral Ganglion Neuron In Vitro. Neural Plast 2016, U.S. Pat. No. 4,280,407 (2016).
57. H. Peretz, A. E. Talpalar, R. Vago, D. Baranes, Superior survival and durability of neurons and astrocytes on 3-dimensional aragonite biomatrices. Tissue Eng 13, 461-472 (2007).
58. Y. Lai, K. Cheng, W. Kisaalita, Three dimensional neuronal cell cultures more accurately model voltage gated calcium channel functionality in freshly dissected nerve tissue. PLoS One 7, e45074 (2012).
59. A. Chandrasekaran, H. X. Avci, A. Ochalek, L. N. Rosingh, K. Molnar, L. Laszlo, T. Bellak, A. Teglasi, K. Pesti, A. Mike, P. Phanthong, O. Biro, V. Hall, N.Kitiyanant, K. H. Krause, J. Kobolak, A. Dinnyes, Comparison of 2D and 3D neural induction methods for the generation of neural progenitor cells from human induced pluripotent stem cells. Stem Cell Res 25, 139-151 (2017).
60. P. D. Wall, M. Lidierth, Five Sources of a Dorsal Root Potential: Their Interactions and Origins in the Superficial Dorsal Horn. Journal of Neurophysiology 78, (1997).
61. R. A. Maile, E. Morgan, J. Bagust, R. J. Walker, Effects of amino acid antagonists on spontaneous dorsal root activity and evoked dorsal horn field potentials in an isolated preparation of rat spinal cord. Int J Neurosci 117, 85-106 (2007).
62. D. M. Gaumann, P. C. Brunet, P. Jirounek, Hyperpolarizing after potentials in C fibers and local anesthetic effects of clonidine and lidocaine. Pharmacology 48, 21-29 (1994).
63. Y. Hayashi, M. Maze, Alpha 2 adrenoceptor agonists and anaesthesia. Br J Anaesth 71, 108-118 (1993).
64. H. S. Fernandes, Clonidine in Anesthesiology: A Brief Review. Biomedical Journal of Scientific & Technical Research 7, (2018).
65. P. Y. Cheng, A. Moriwaki, J. B. Wang, G. R. Uhl, V. M. Pickel, Ultrastructural localization of μ-opioid receptors in the superficial layers of the rat cervical spinal cord: extrasynaptic localization and proximity to LeuS-enkephalin. Brain Research 731, 141-154 (1996).
66. R. Bardoni, V. L. Tawfik, D. Wang, A. Francois, C. Solorzano, S. A. Shuster, P. Choudhury, C. Betelli, C. Cassidy, K. Smith, J. C. de Nooij, F. Mennicken, D. O'Donnell, B. L. Kieffer, C. J. Woodbury, A. I. Basbaum, A. B. MacDermott, G. Scherrer, Delta opioid receptors presynaptically regulate cutaneous mechanosensory neuron input to the spinal cord dorsal horn. Neuron 81, 1312-1327 (2014).
67. D. Zhang, M. Pekkanen-Mattila, M. Shahsavani, A. Falk, A. I. Teixeira, A. Herland, A 3D Alzheimer's disease culture model and the induction of P21-activated kinase mediated sensing in iPSC derived neurons. Biomaterials 35, 1420-1428 (2014).
68. L. D'Aiuto, J. Naciri, N. Radio, S. Tekur, D. Clayton, G. Apodaca, R. Di Maio, Y. Zhi, P. Dimitrion, P. Piazza, M. Demers, J. Wood, C. Chu, J. Callio, L. McClain, R. Yolken, J. McNulty, P. Kinchington, D. Bloom, V. Nimgaonkar, Generation of three-dimensional human neuronal cultures: application to modeling CNS viral infections. Stem Cell Res Ther 9, 134 (2018).
69. S. Bosi, R. Rauti, J. Laishram, A. Turco, D. Lonardoni, T. Nieus, M. Prato, D. Scaini, L. Ballerini, From 2D to 3D: novel nanostructured scaffolds to investigate signalling in reconstructed neuronal networks. Sci Rep 5, 9562 (2015).
70. J. L. Bourke, A. F. Quigley, S. Duchi, C. D. O'Connell, J. M. Crook, G. G. Wallace, M. J. Cook, R. M. I. Kapsa, Three-dimensional neural cultures produce networks that mimic native brain activity. Journal of Tissue Engineering and Regenerative Medicine 12, 490-493 (2017).
71. W. A. Anderson, A. Bosak, H. T. Hogberg, T. Hartung, M. J. Moore, Advances in 3D neuronal microphysiological systems: towards a functional nervous system on a chip. In Vitro Cell Dev Biol Anim, (2021).
72. D. A. Bowser, M. J. Moore, Biofabrication of neural microphysiological systems using magnetic spheroid bioprinting. Biofabrication 12, 015002 (2019).
73. A. D. Sharma, L. McCoy, E. Jacobs, H. Willey, J. Q. Behn, H. Nguyen, B. Bolon, J. L. Curley, M. J. Moore, Engineering a 3D functional human peripheral nerve in vitro using the Nerve-on-a-Chip platform. Sci Rep 9, 8921 (2019).

1. A composition comprising a first spheroid of cells comprising one or a combination of cells and/or tissues chosen from: a neuronal cell, an astrocyte and a glial cell.

2. The composition of claim 1, wherein the first spheroid further comprises a tissue derived from a spinal cord of a mammal.

3. The composition of claim 1, wherein the cells are derived from human spinal cord tissue.

4. The composition of claim 1, wherein the cells are organized in a tissue.

5. The composition of claim 1 further comprising a second spheroid of cells comprising one or a combination of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, an astrocyte, an oligodendrocyte, a Schwann cell, a microglial cell, an ependymal cell, a radial glial cell, a satellite cell, an enteric glial cell, a pituicyte, and combinations thereof.

6.-9. (canceled)

10. The composition of claim 5, wherein the first spheroid is in electrical communication with the second spheroid by a three-dimensional bundle of axons.

11. The composition of claim 10, wherein the bundle of axons comprises from about 3 to about 125 axons.

12. The composition of claim 11, wherein the axons are in a parallel orientation and wherein the bundles comprise from about 10 to about 120 axons.

13. The composition of claim 5, wherein the first spheroid has a diameter from about 100 microns to about 800 microns and the second spheroid of cells has a diameter from about 100 microns to about 800 microns.

14. (canceled)

15. The composition of claim 5, wherein the second spheroid of cells is optically excitable by a wavelength of light from about 400 to about 500 nanometers.

16.-34. (canceled)

35. A method of measuring or detecting a recording in an in vitro synapse within a composition comprising: (i) a first spheroid of cells in electrical communication with a second spheroid of cells; (ii) a first electrode proximate to the first spheroid; and (iii) a second electrode proximate to the second spheroid, the method comprising:
(a) applying a voltage across the first and second electrodes at the first electrode for a sufficient time period and with a sufficient voltage to generate a detectable control waveform from the second electrode;
(b) detecting the control waveform from the second electrode.

36.-38. (canceled)

39. The method of claim 35, wherein the voltage of step (a) is from about 1 to about 50 Volts.

40. The method of claim 35, wherein a second voltage is applied across the first and second electrodes at the first electrode after exposing the spheroids to one or a plurality of test agents.

41. The method of claim 35 further comprising applying a second voltage across the first and second spheroids at the first electrode in the presence or absence of a test agent and a step of detecting a waveform associated with the field potential of the synapse in the presence of the test agent.

42. The method of any of claim 35, wherein the the first spheroid of cells comprises one or a combination of cells and/or tissues chosen from: a neuronal cell, an astrocyte and a glial cell, and the second spheroid of cells comprises one or a combination of cells chosen from: a glial cell, an embryonic cell, a mesenchymal stem cell, a cell derived from an induced pluripotent stem cell, a sympathetic neuron, a parasympathetic neuron, a spinal motor neurons, a central nervous system neuron, a peripheral nervous system neuron, an enteric nervous system neurons, a motor neuron, a sensory neuron, a cholinergic neuron, a GABAergic neuron, a glutamatergic neuron, a dopaminergic neuron, a serotonergic neuron, an interneuron, an adrenergic neuron, a trigeminal ganglion, an astrocyte, an oligodendrocyte, a Schwann cell, a microglial cell, an ependymal cell, a radial glial cell, a satellite cell, an enteric glial cell, a pituicyte, and combinations thereof.

43.-47. (canceled)

48. A method of manufacturing a three-dimensional culture of a synapse comprising one or a plurality of spheroids in a culture vessel comprising a solid substrate defining an interior chamber with at least a first and second cavity connected by at least one channel positioned therebetween, said method comprising:
(a) contacting a first plurality of neuronal cells from a mammal with the solid substrate in the first cavity of the interior chamber; and (b) contacting a second plurality of neuronal cells from a mammal in the second cavity of the interior chamber; and (c) allowing a time sufficient for the first plurality of neuronal cells to organize into a first spheroid and the second plurality of neuronal cells to organize into a second spheroid; and
(d) applying a cell medium into the culture vessel with a volume of cell medium sufficient to cover the first and second spheroid;
wherein the first spheroid is from dorsal root ganglia and the second spheroid is from the spinal cord ganglia.

49. The method of claim 48, wherein the first spheroid comprising the dorsal root ganglia comprises from about 10,000 cells to about 30,000 cells.

50. The method of claim 48, wherein the second spheroid comprising the spinal cord neurons comprises from about 10,000 cells to about 30,000 cells.

51.-66. (canceled)

67. The method of claim 48, wherein the neuronal cells are human.

68. The method of 67, wherein the neuronal cells are derived from stem cells.
